SEQUENCE LISTING (99 sequences)

SEQ ID NO: 1 - DNA, 20 bp, Artificial Sequence
  synthetic core RYGGYY class immune modulatory sequence (IMS) I1
tccatgtggttcctgaccat

SEQ ID NO: 2 - DNA, 15 bp, Artificial Sequence
  synthetic core RYGGYY class immune modulatory sequence (IMS) I5
ggtgcatggttgcag

SEQ ID NO: 3 - DNA, 22 bp, Artificial Sequence
  synthetic core RYGGYY class immune modulatory sequence (IMS) I6
tggtggttttggccttttggcc

SEQ ID NO: 4 - DNA, 22 bp, Artificial Sequence
  synthetic core RYGGYY class immune modulatory sequence (IMS) I7
tgactgtggtggccacagatga

SEQ ID NO: 5 - DNA, 15 bp, Artificial Sequence
  synthetic core RYGGYY class immune modulatory sequence (IMS) I19
ccatgtggttatttt

SEQ ID NO: 6 - DNA, 16 bp, Artificial Sequence
  synthetic core RYGGYY class immune modulatory sequence (IMS) I20
ctgtggtggttagaga

SEQ ID NO: 7 - DNA, 13 bp, Artificial Sequence
  synthetic core RYGGYY class immune modulatory sequence (IMS) I18.5
ccgtggttatggt

SEQ ID NO: 8 - DNA, 14 bp, Artificial Sequence
  synthetic core RYGGYY class immune modulatory sequence (IMS) I18.13
cctgtggccatggt

SEQ ID NO: 9 - DNA, 15 bp, Artificial Sequence
  synthetic core RYGGYY class immune modulatory sequence (IMS) I18.17
ccatgtggttatggt

SEQ ID NO: 10 - DNA, 15 bp, Artificial Sequence
  synthetic core RYGGYY class immune modulatory sequence (IMS) I18.18
ccaagtggttatggt

SEQ ID NO: 11 - DNA, 22 bp, Artificial Sequence
  synthetic core RYGGYY class immune modulatory sequence (IMS) GpG.1
tgactgtggtggttagagatga

SEQ ID NO: 12 - DNA, 16 bp, Artificial Sequence
  synthetic core RYGGYY class immune modulatory sequence (IMS) GpG.2
ctgtggtggttagaga

SEQ ID NO: 13 - DNA, 14 bp, Artificial Sequence
  synthetic core RYGGYY class immune modulatory sequence (IMS) GpG.3
ctctgtggttagag

SEQ ID NO: 14 - DNA, 14 bp, Artificial Sequence
  synthetic core RYGGYY class immune modulatory sequence (IMS) GpG.4
ctctgtggttcccc

SEQ ID NO: 15 - DNA, 14 bp, Artificial Sequence
  synthetic core RYGGYY class immune modulatory sequence (IMS) GpG.5
gagagtggttagag

SEQ ID NO: 16 - DNA, 14 bp, Artificial Sequence
  synthetic core RYGGYY class immune modulatory sequence (IMS) GpG.6
gagagtggttcccc

SEQ ID NO: 17 - DNA, 14 bp, Artificial Sequence
  synthetic core RYGGYY class immune modulatory sequence (IMS) GpG.7
ccgagtggttacgg

SEQ ID NO: 18 - DNA, 14 bp, Artificial Sequence
  synthetic core RYGGYY class immune modulatory sequence (IMS) GpG.8
tggcgtggcctggc

SEQ ID NO: 19 - DNA, 14 bp, Artificial Sequence
  synthetic core RYGGYY class immune modulatory sequence (IMS) GpG.9
aaaagtggttcccc

SEQ ID NO: 20 - DNA, 14 bp, Artificial Sequence
  synthetic core RYGGYY class immune modulatory sequence (IMS) GpG.10
aaaagtggcctttt

SEQ ID NO: 21 - DNA, 14 bp, Artificial Sequence
  synthetic core RYGGYY class immune modulatory sequence (IMS) GpG.11
aaaagtggcctttt

SEQ ID NO: 22 - DNA, 14 bp, Artificial Sequence
  synthetic core RYGGYY class immune modulatory sequence (IMS) GpG.12
aaaagtggttaaaa

SEQ ID NO: 23 - DNA, 22 bp, Artificial Sequence
  synthetic core RYGGYY class immune modulatory sequence (IMS) GpG.cc
tgactgtggtggccagagatga

SEQ ID NO: 24 - DNA, 12 bp, Artificial Sequence
  synthetic core RYGGYY class immune modulatory sequence (IMS) I41
cctgtggttcct

SEQ ID NO: 25 - DNA, 23 bp, Artificial Sequence
  synthetic poly G + RYGGY class immune modulatory sequence (IMS) I2
ttatgtggttcctgaccaggggg

SEQ ID NO: 26 - DNA, 32 bp, Artificial Sequence
  synthetic poly G + RYGGY class immune modulatory sequence (IMS) I3
attatggggtgtggttttccacaccccggggg

SEQ ID NO: 27 - DNA, 27 bp, Artificial Sequence
  synthetic poly G + RYGGY class immune modulatory sequence (IMS) I4
attatggggtgtggttttccacacccc

SEQ ID NO: 28 - DNA, 32 bp, Artificial Sequence; identical to SEQ ID NO: 26
  synthetic poly G + RYGGY class immune modulatory sequence (IMS) I11

SEQ ID NO: 29 - DNA, 24 bp, Artificial Sequence
  synthetic poly G + RYGGY class immune modulatory sequence (IMS) I13
tgactgtggtggttagagatgggt

SEQ ID NO: 30 - DNA, 31 bp, Artificial Sequence
  synthetic poly G + RYGGY class immune modulatory sequence (IMS) I14
tgactgtggtggttagagatgggttttgggt

SEQ ID NO: 31 - DNA, 26 bp, Artificial Sequence
  synthetic poly G + RYGGY class immune modulatory sequence (IMS) I16
tgtggttacagtggttgtggttgggg

SEQ ID NO: 32 - DNA, 16 bp, Artificial Sequence
  synthetic poly G + RYGGY class immune modulatory sequence (IMS) I17
ccatgtggttatgggg

SEQ ID NO: 33 - DNA, 16 bp, Artificial Sequence
  synthetic human poly G + RYGGY class immune modulatory sequence (IMS) I18
ccatgtggttatgggt

SEQ ID NO: 34 - DNA, 22 bp, Artificial Sequence
  synthetic poly G + RYGGY class immune modulatory sequence (IMS) I21
tggtggttttgggcgcgcgccg

SEQ ID NO: 35 - DNA, 20 bp, Artificial Sequence
  synthetic poly G + RYGGY class immune modulatory sequence (IMS) I23
ggtgcatggttgcagggggg

SEQ ID NO: 36 - DNA, 16 bp, Artificial Sequence
  synthetic poly G + RYGGY class immune modulatory sequence (IMS) I27
cctcatggttgagggg

SEQ ID NO: 37 - DNA, 20 bp, Artificial Sequence
  synthetic poly G + RYGGY class immune modulatory sequence (IMS) I28
ggggccatgtggttatgggg

SEQ ID NO: 38 - DNA, 20 bp, Artificial Sequence
  synthetic poly G + RYGGY class immune modulatory sequence (IMS) I29
tgctgcacatggttgagggg

SEQ ID NO: 39 - DNA, 29 bp, Artificial Sequence
  synthetic poly G + RYGGY class immune modulatory sequence (IMS) I30
ggggggtgctgcacagtggttcagggggg

SEQ ID NO: 40 - DNA, 16 bp, Artificial Sequence
  synthetic poly G + RYGGY class immune modulatory sequence (IMS) I31
cctcatggccaagggg

SEQ ID NO: 41 - DNA, 17 bp, Artificial Sequence
  synthetic poly G + RYGGY class immune modulatory sequence (IMS) I33
tgggtgtggttatgggt

SEQ ID NO: 42 - DNA, 16 bp, Artificial Sequence
  synthetic poly G + RYGGY class immune modulatory sequence (IMS) I36
ccacgtggccatgggt

SEQ ID NO: 43 - DNA, 16 bp, Artificial Sequence
  synthetic poly G + RYGGY class immune modulatory sequence (IMS) I39
ccatgtggttatgggt

SEQ ID NO: 44 - DNA, 12 bp, Artificial Sequence
  synthetic poly G + RYGGY class immune modulatory sequence (IMS) I40
tggtggttgggt

SEQ ID NO: 45 - DNA, 15 bp, Artificial Sequence
  synthetic poly G + RYGGY class immune modulatory sequence (IMS) I18.2
cctgtggttatgggt

SEQ ID NO: 46 - DNA, 16 bp, Artificial Sequence
  synthetic poly G + RYGGY class immune modulatory sequence (IMS) I18.3
tcctgtggttatgggt

SEQ ID NO: 47 - DNA, 16 bp, Artificial Sequence
  synthetic poly G + RYGGY class immune modulatory sequence (IMS) I18.4
tggtgtggttatgggt

SEQ ID NO: 48 - DNA, 12 bp, Artificial Sequence
  synthetic poly G + RYGGY class immune modulatory sequence (IMS) I18.6
ccgtggttgggt

SEQ ID NO: 49 - DNA, 13 bp, Artificial Sequence
  synthetic poly G + RYGGY class immune modulatory sequence (IMS) I18.7
cagtggcctgggt

SEQ ID NO: 50 - DNA, 14 bp, Artificial Sequence
  synthetic poly G + RYGGY class immune modulatory sequence (IMS) I18.8
aaagtggcctgggt

SEQ ID NO: 51 - DNA, 13 bp, Artificial Sequence
  synthetic poly G + RYGGY class immune modulatory sequence (IMS) I18.9
cagtggcctgggt

SEQ ID NO: 52 - DNA, 14 bp, Artificial Sequence
  synthetic poly G + RYGGY class immune modulatory sequence (IMS) I18.10
ccagtggcctgggt

SEQ ID NO: 53 - DNA, 14 bp, Artificial Sequence
  synthetic poly G + RYGGY class immune modulatory sequence (IMS) I18.11
ccagtggcctgggt

SEQ ID NO: 54 - DNA, 19 bp, Artificial Sequence
  synthetic poly G + RYGGY class immune modulatory sequence (IMS) I18.14
aaaagtggcctttgggtcc

SEQ ID NO: 55 - DNA, 16 bp, Artificial Sequence
  synthetic poly G + RYGGY class immune modulatory sequence (IMS) I18.15
ccaagtggttatgggt

SEQ ID NO: 56 - DNA, 16 bp, Artificial Sequence
  synthetic poly G + RYGGY class immune modulatory sequence (IMS) I18.16
gcatgtggttatgggt

SEQ ID NO: 57 - DNA, 16 bp, Artificial Sequence
  synthetic poly G + RYGGY class immune modulatory sequence (IMS) I18.19
aaaagtggttatgggt

SEQ ID NO: 58 - DNA, 22 bp, Artificial Sequence
  synthetic multiple core RYGGYY motifs class immune modulatory sequence (IMS) I8
tgtggttacagcggttgtggcc

SEQ ID NO: 59 - DNA, 28 bp, Artificial Sequence
  synthetic multiple core RYGGYY motifs class immune modulatory sequence (IMS) I9
tggtggtgtggccacagtggttgtggcc

SEQ ID NO: 60 - DNA, 22 bp, Artificial Sequence
  synthetic multiple core RYGGYY motifs class immune modulatory sequence (IMS) I10
tggtggtgtggccacagtggtt

SEQ ID NO: 61 - DNA, 22 bp, Artificial Sequence
  synthetic multiple core RYGGYY motifs class immune modulatory sequence (IMS) I12
tgtggttacagcggttgtggtt

SEQ ID NO: 62 - DNA, 22 bp, Artificial Sequence
  synthetic multiple core RYGGYY motifs class immune modulatory sequence (IMS) I15
tgtggttacagtggttgtggtt

SEQ ID NO: 63 - DNA, 24 bp, Artificial Sequence
  synthetic multiple core RYGGYY motifs class immune modulatory sequence (IMS) I22
tggtggttttgtggttttgtggtt

SEQ ID NO: 64 - DNA, 42 bp, Artificial Sequence
  synthetic multiple core RYGGYY motifs class immune modulatory sequence (IMS) I26
ggttggtgtggttggacagtggttgttggttggtgtggttgg

SEQ ID NO: 65 - DNA, 28 bp, Artificial Sequence
  synthetic multiple core RYGGYY motifs class immune modulatory sequence (IMS) I34
tggtggtgtggccacagtggccgtggcc

SEQ ID NO: 66 - DNA, 28 bp, Artificial Sequence
  synthetic multiple core RYGGYY motifs class immune modulatory sequence (IMS) I37
tgctgctgtggccacagtggccgtggcc

SEQ ID NO: 67 - DNA, 36 bp, Artificial Sequence
  synthetic multiple core RYGGYY motifs + poly G class immune modulatory sequence (IMS) I35
tggtggtgtggccacagtggccacagtggcctgggt

SEQ ID NO: 68 - DNA, 33 bp, Artificial Sequence
  synthetic multiple core RYGGYY motifs + poly G class immune modulatory sequence (IMS) I38
tgctgctgtggccacagtggccgtggcctgggt

SEQ ID NO: 69 - DNA, 22 bp, Artificial Sequence
  synthetic multiple core RYGGYY motifs + poly G class immune modulatory sequence (IMS) I42
ccagtggcccagtggcctgggt

SEQ ID NO: 70 - DNA, 21 bp, Artificial Sequence
  synthetic multiple core RYGGYY motifs + poly G class immune modulatory sequence (IMS) I43
cagtggcccagtggcctgggt

SEQ ID NO: 71 - DNA, 29 bp, Artificial Sequence
  synthetic core RYGGYY + G-tetrad class immune modulatory sequence (IMS) I24
ccatgtggttatggtgtggtgtggtgtgg

SEQ ID NO: 72 - DNA, 31 bp, Artificial Sequence
  synthetic core RYGGYY + G-tetrad class immune modulatory sequence (IMS) I25
tggtggtgtggcctggtgtggtgtggtgtgg

SEQ ID NO: 73 - DNA, 16 bp, Artificial Sequence
  synthetic immune modulatory sequence (IMS)
ccatgtggttatgggt

SEQ ID NO: 74 - DNA, 16 bp, Artificial Sequence
  synthetic I18 derived immune modulatory sequence (IMS) I18.M1
ccatctggttatgggt

SEQ ID NO: 75 - DNA, 16 bp, Artificial Sequence
  synthetic I18 derived immune modulatory sequence (IMS) I18.M2
ccatcaggttatgggt

SEQ ID NO: 76 - DNA, 16 bp, Artificial Sequence
  synthetic I18 derived immune modulatory sequence (IMS) I18.M3 with poly G mutation
ccatgtggttatgagt

SEQ ID NO: 77 - DNA, 16 bp, Artificial Sequence
  synthetic I18 derived immune modulatory sequence (IMS) I18.M4 with poly G mutation
ccatgtggttatgtgt

SEQ ID NO: 78 - DNA, 16 bp, Artificial Sequence
  synthetic I18 derived immune modulatory sequence (IMS) I18.M5 with poly G mutation
ccatgtggttataggt

SEQ ID NO: 79 - DNA, 16 bp, Artificial Sequence
  synthetic I18 derived immune modulatory sequence (IMS) I18.M6 with poly G mutation
ccatgtggttatggat

SEQ ID NO: 80 - DNA, 16 bp, Artificial Sequence
  synthetic I18 derived immune modulatory sequence (IMS) I18.M7
ccatgtggaaatgggt

SEQ ID NO: 81 - DNA, 16 bp, Artificial Sequence
  synthetic I18 derived immune modulatory sequence (IMS) I18.M8 with poly G mutation
ccatgtggttatgaat

SEQ ID NO: 82 - DNA, 16 bp, Artificial Sequence
  synthetic I18 derived immune modulatory sequence (IMS) I18.M9
ccatgaggttatgggt

SEQ ID NO: 83 - DNA, 16 bp, Artificial Sequence
  synthetic I18 derived immune modulatory sequence (IMS) I18.M10 with mutation 5′ to 5′-GTGGTT-3′ core hexamer
ggatgtggttatgggt

SEQ ID NO: 84 - DNA, 16 bp, Artificial Sequence
  synthetic I18 derived immune modulatory sequence (IMS) I18.M11 with mutation 5′ to 5′-GTGGTT-3′ core hexamer
gagtgtggttatgggt

SEQ ID NO: 85 - DNA, 16 bp, Artificial Sequence
  synthetic I18 derived immune modulatory sequence (IMS) I18.M12 with mutation 5′ to 5′-GTGGTT-3′ core hexamer
aggtgtggttatgggt

SEQ ID NO: 86 - DNA, 17 bp, Artificial Sequence
  synthetic I18 derived immune modulatory sequence (IMS) I18.M13 with addition of nucleotide between 5′-GTGGTT-3′ core hexamer and poly G
ccatgtggttaatgggt

SEQ ID NO: 87 - DNA, 18 bp, Artificial Sequence
  synthetic I18 derived immune modulatory sequence (IMS) I18.M14 with addition of nucleotides between 5′-GTGGTT-3′ core hexamer and poly G
ccatgtggttaaatgggt

SEQ ID NO: 88 - DNA, 19 bp, Artificial Sequence
  synthetic I18 derived immune modulatory sequence (IMS) I18.M15 with addition of nucleotides between 5′-GTGGTT-3′ core hexamer and poly G
ccatgtggttaaaatgggt

SEQ ID NO: 89 - DNA, 20 bp, Artificial Sequence
  synthetic I18 derived immune modulatory sequence (IMS) I18.M16 with addition of nucleotides between 5′-GTGGTT-3′ core hexamer and poly G
ccatgtggttaaaaatgggt

SEQ ID NO: 90 - DNA, 16 bp, Artificial Sequence
  synthetic I18 derived immune modulatory sequence (IMS) I18.M17 with addition of nucleotides between 5′-GTGGTT-3′ core hexamer and poly G
ccatgtggttaagggt

SEQ ID NO: 91 - DNA, 16 bp, Artificial Sequence
  synthetic mouse poly G + RYGGY class immune modulatory sequence (IMS) I18
ccataaggttatgggt

SEQ ID NO: 92 - DNA, 16 bp, Artificial Sequence
  synthetic immune modulatory sequence (IMS) M49
ccatgtgcccatgggt

SEQ ID NO: 93 - DNA, 31 bp, Artificial Sequence
  synthetic immune modulatory sequence (IMS) motif with core hexamer and flanking sequences
tgactgtgccnnrynnyynngggagagatga

SEQ ID NO: 94 - DNA, 30 bp, Artificial Sequence
  synthetic poly G region flanking immune modulatory sequence (IMS)
gggggggggggggggggggggggggggggg

SEQ ID NO: 95 - DNA, 16 bp, Artificial Sequence
  synthetic I18 derived immune modulatory sequence (IMS) I18.M7
ccatgtggaaatgggt

SEQ ID NO: 96 - DNA, 16 bp, Artificial Sequence
  synthetic I18 derived immune modulatory sequence (IMS) I18.M49
ccatgtggccctgggt

SEQ ID NO: 97 - DNA, 16 bp, Artificial Sequence
  synthetic I18 derived immune modulatory sequence (IMS) I18.M51
ccatgtggaaaagggt

SEQ ID NO: 98 - DNA, 16 bp, Artificial Sequence
  synthetic I18 derived immune modulatory sequence (IMS) I18.M52
ccatgtggaaaaggga

SEQ ID NO: 99 - DNA, 16 bp, Artificial Sequence
  synthetic I18 derived immune modulatory sequence (IMS) I18.M53
ccatgtgcccaaggga

(19) United States
(12) Patent Application Publication
(10) Pub. No.: US 2010/0130593 A1
(43) Pub. Date: May 27, 2010

(54) METHODS AND IMMUNE MODULATOR NUCLEIC ACID COMPOSITIONS FOR PREVENTING AND TREATING DISEASE

(75) Inventors: Hideki Garren, Palo Alto, CA (US); Michael Leviten, Palo Alto, CA (US); Nanette Solvason, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: BAYHILL THERAPEUTICS, INC., Palo Alto, CA (US)

(21) Appl. No.: 12/304,459

(22) PCT Filed: Jun. 13, 2007
(86) PCT No.: PCT/US2007/071130

§ 371 Date: Feb. 8, 2010

Related U.S. Application Data
(60) Provisional application No. 60/813,538, filed on Jun. 13, 2006.
(60) Provisional application No. 60/849,901, filed on Oct. 5, 2006.

(51) Int. Cl. A61K 31/711; A61P 37/04

(52) U.S. Cl. 514/44 R

(57) ABSTRACT

This invention relates to methods and compositions for treating or preventing disease comprising the administration of immune modulatory nucleic acids having one or more immune modulatory sequences (IMSs). The invention further relates to the means and methods for the identification of the IMSs for preventing or treating disease, more particularly the treatment and prevention of autoimmune or inflammatory diseases. The invention also relates to the treatment or prevention of disease comprising the administration of the immune modulatory nucleic acids alone or in combination with a polynucleotide encoding self-antigen(s), -proteins(s), -polypeptide(s) or -peptide(s). The present invention also relates to methods and compositions for treating diseases in a subject associated with one or more self-antigen(s), self-proteins(s), -polypeptide(s) or -peptide(s) that are present in the subject and involved in a non-physiological state.

### CROSS-REFERENCES TO RELATED APPLICATIONS

This application claims the benefit of U.S. Provisional Patent Application 60/813,538, filed Jun. 13, 2006 and U.S. Provisional Patent Application 60/849,901, filed Oct. 5, 2006, the entire disclosures of both of which are hereby incorporated herein by reference for all purposes.

### BACKGROUND OF THE INVENTION

1. Field of the Invention

This invention relates to methods and compositions for treating or preventing disease. The methods comprise the administration of immune modulatory sequences. The invention further relates to improved immune modulatory sequences for preventing or treating disease, more particularly the treatment and prevention of autoimmune disease or inflammatory diseases. The invention also relates to the treatment or prevention of disease comprising the administration of the immune modulatory sequences alone. The invention also relates to the treatment or prevention of disease comprising the administration of the immune modulatory sequences in combination with a polynucleotide encoding self-antigen(s), -protein(s), -polypeptide(s) or -peptide(s). For example, the immune modulatory sequences of the invention can be incorporated into expression vectors expressing a self-antigen. The invention further relates to the treatment or prevention of disease comprising the administration of the immune modulatory sequences in combination with self-molecules, such as self-lipids, self-antigen(s), self-protein(s), self-peptide(s), self-polypeptide(s), self-glycolipid(s), self-carbohydrate(s), self-glycoprotein(s), and posttranslationally-modified self-protein(s), peptide(s), polypeptide(s), or glycoprotein(s). The invention also relates to the treatment or prevention of disease comprising the administration of the immune modulatory sequences in combination with one or more additional immune modulatory therapeutics.

The present invention also relates to methods and compositions for treating diseases in a subject associated with one or more self-antigen(s), -protein(s), -polypeptide(s) or -peptide(s) that are present in the subject and involved in a non-physiological state. The present invention also relates to methods and compositions for preventing diseases in a subject associated with one or more self-antigen(s), -protein(s), -polypeptide(s) or -peptide(s) that are present in the subject and involved in a non-physiological state. The invention also relates to the administration of a combined therapy comprising an immune modulatory sequence and a polynucleotide encoding a self-antigen(s), -protein(s), -polypeptide(s) or -peptide(s) present in a non-physiological state and associated with a disease. The invention also relates to modulating an immune response to self-molecule(s) present in an animal and involved in a non-physiological state and associated with a disease. The invention is more particularly related to the methods and compositions for treating or preventing autoimmune diseases associated with one or more self-molecule(s) present in the animal in a non-physiological state such as in multiple sclerosis (MS), rheumatoid arthritis (RA), insulin dependent diabetes mellitus (IDDM), autoimmune uveitis (AU), primary biliary cirrhosis (PBC), myasthenia gravis (MG), Sjogren's syndrome, pemphigus vulgaris (PV), scleroderma, pernicious anemia, systemic lupus erythematosus (SLE) and Grave's disease. The invention is further particularly related to other diseases associated with one or more self-molecule(s) present in the animal in a non-physiological state such as osteoarthritis, spinal cord injury, peptic ulcer disease, gout, migraine headaches, hyperlipidemia and coronary artery disease.

2. Background

### Autoimmune Disease

Autoimmune disease is a disease caused by adaptive immunity that becomes misdirected at healthy cells and/or tissues of the body. Autoimmune disease affects 3% of the U.S. population, and likely a similar percentage of the industrialized world population (Jacobson et al., Clin Immunol Immunopathol, 84, 223-43, 1997). Autoimmune diseases are characterized by T and B lymphocytes that aberrantly target self-molecules, including but not limited to self-lipids, self-antigen(s), self-protein(s), self-peptide(s), self-polypeptide(s), self-glycolipid(s), self-carbohydrate(s), self-glycoprotein(s), and posttranslationally-modified self-protein(s), peptide(s), polypeptide(s), or glycoprotein(s), and derivatives thereof, thereby causing injury and or malfunction of an organ, tissue, or cell-type within the body (for example, pancreas, brain, thyroid or gastrointestinal tract) to cause the clinical manifestations of the disease (Marrack et al., Nat Med, 7, 899-905, 2001). Autoimmune diseases include diseases that affect specific tissues as well as diseases that can affect multiple tissues. This may, in part, for some diseases depend on whether the autoimmune responses are directed to a self molecule antigen confined to a particular tissue or to a self molecule antigen that is widely distributed in the body. The characteristic feature of tissue-specific autoimmunity is the selective targeting or effect on a single tissue or individual cell type. Nevertheless, certain autoimmune diseases that target ubiquitous self molecules antigens can also affect specific tissues. For example, in polymyositis the autoimmune response targets the ubiquitous protein histidyl-tRNA synthetase, yet the clinical manifestations primarily involved autoimmune destruction of muscle.

The immune system employs a highly complex mechanism designed to generate responses to protect mammals against a variety of foreign pathogens while at the same time preventing responses against self-antigen(s). In addition to deciding whether to respond (antigen specificity), the immune system must also choose appropriate effector functions to deal with each pathogen (effector specificity). A cell critical in mediating and regulating these effector functions is the CD4+ T cell. Furthermore, it is the elaboration of specific cytokines from CD4+ T cells that appears to be one of the major mechanisms by which T cells mediate their functions. Thus, characterizing the types of cytokines made by CD4+ T cells as well as how their secretion is controlled is extremely important in understanding how the immune response is regulated.

The characterization of cytokine production from long-term mouse CD4+ T cell clones was first published more than 10 years ago (Mosmann et al., J. Immunol., 136:2348-2357, 1986). In these studies, it was shown that CD4+ T cells produced two distinct patterns of cytokine production, which were designated T helper 1 (Th1) and T helper 2 (Th2). Th1 cells were found to selectively produce interleukin-2 (IL-2), interferon-gamma (IFN-gamma) and lymphotoxin (LT), while Th2 clones selectively produced IL-4, IL-5, IL-6, and IL-13 (Cherwinski et al., J. Exp. Med., 169:1229-1244, 1987). Somewhat later, additional cytokines, IL-9 and IL-10, were isolated from Th2 clones (Van Snick et al., J. Exp. Med., 169:363-368, 1989; Fiorentino et al., J. Exp. Med., 170:2081-2095, 1989). Finally, additional cytokines, such as IL-3, granulocyte macrophage colony-stimulating factor (GM-CSF), and tumor necrosis factor-alpha (TNF-alpha) were found to be secreted by both Th1 and Th2 cells.

Autoimmune disease encompasses a wide spectrum of diseases that can affect many different organs and tissues within the body as outlined in the table below. See, e.g., Paul W. E. (ed. 2003) Fundamental Immunology (5th Ed.) Lippincott Williams & Wilkins; ISBN-10: 0781735149, ISBN-13: 978-0781735148; Rose and Mackay (eds. 2006) The Autoimmune Diseases (4th ed.) Academic Press, ISBN-10: 0125959613, ISBN-13: 978-0125959612; Erkan, et al. (eds. 2004) The Neurologic Involvement in Systemic Autoimmune Diseases, Volume 3 (Handbook of Systemic Autoimmune Diseases) Elsevier Science, ISBN-10: 0444516514, ISBN-13: 978-0444516510; and Richter, et al. (eds. 2003) Treatment of Autoimmune Disorders, Springer, ISBN-10: 3211837728, ISBN-13: 978-3211837726.

Current therapies for human autoimmune disease include glucocorticoids, cytotoxic agents, and recently developed biological therapeutics. In general, the management of human systemic autoimmune disease is empirical and unsatisfactory. For the most part, broadly immunosuppressive drugs, such as corticosteroids, are used in a wide variety of severe autoimmune and inflammatory disorders. In addition to corticosteroids, other immunosuppressive agents are used in management of the systemic autoimmune diseases. Cyclophosphamide is an alkylating agent that causes profound depletion of both T- and B-lymphocytes and impairment of cell-mediated immunity. Cyclosporine, tacrolimus, and mycophenolate mofetil are natural products with specific properties of T-lymphocyte suppression, and they have been used to treat SLE, RA and, to a limited extent, in vasculitis and myositis. These drugs are associated with significant renal toxicity. Methotrexate is also used as a “second line” agent in RA, with the goal of reducing disease progression. It is also used in polymyositis and other connective-tissue diseases. Other approaches that have been tried include monoclonal antibodies intended to block the action of cytokines or to deplete lymphocytes. See, Fox, D. A. Am. J. Med., 99:82-88, 1995. Treatments for MS include interferon Beta and copolymer 1, which reduce relapse rate by 20-30% and only have a modest impact on disease progression. MS is also treated with immunosuppressive agents including methylprednisolone, other steroids, methotrexate, cladribine and cyclophosphamide. These immunosuppressive agents have minimal efficacy in treating MS. Current therapy for RA utilizes agents that non-specifically suppress or modulate immune function such as methotrexate, sulfasalazine, hydroxychloroquine, leflunamide, prednisone, as well as the recently developed TNF alpha antagonists etanercept and infliximab (Moreland et al., J Rheumatol, 28, 1431-52, 2001). Etanercept and infliximab globally block TNF alpha, making patients more susceptible to death from sepsis, aggravation of chronic mycobacterial infections, and development of demyelinating events.

In the case of organ-specific autoimmunity, a number of different therapeutic approaches have been tried. Soluble protein antigens have been administered systemically to inhibit the subsequent immune response to that antigen. Such therapies include delivery of myelin basic protein, its dominant peptide, or a mixture of myelin proteins to animals with experimental autoimmune encephalomyelitis (EAE) and humans with multiple sclerosis (Brocke et al., Nature, 379, 343-6, 1996; Critchfield et al., Science, 263, 1139-43, 1994); Weiner et al., Annu Rev Immunol, 12, 809-37, (1994)); administration of type II collagen or a mixture of collagen proteins to animals with collagen-induced arthritis and humans with rheumatoid arthritis (Gumanovskaya et al., Immunology, 97, 466-73, 1999; McKown et al., Arthritis Rheum, 42, 1204-8, 1999; Trentham et al., Science, 261, 1727-30, 1993); delivery of insulin to animals and humans with autoimmune diabetes (Pozzilli and Gisella Cavallo, Diabetes Metab Res Rev, 16, 306-7, 2000); and delivery of S-antigen to animals and humans with autoimmune uveitis (Nussenblatt et al., Am J Ophthalmol, 123, 583-92, 1997). A problem associated with this approach is T-cell unresponsiveness induced by systemic injection of antigen. Another approach is the attempt to design rational therapeutic strategies for the systemic administration of a peptide antigen based on the specific interaction between the T-cell receptors and peptides bound to major histocmpatibility (MHC) molecules. One study using the peptide approach in an animal model of diabetes resulted in the development of antibody production to the peptide (Hurtenbach U. et al., J Exp. Med, 177:1499, 1993). Another approach is the administration of TCR peptide immunization. See, for example, Vandenbark A A et al., Nature, 341:541, 1989. Still another approach is the induction of oral tolerance by ingestion of peptide or protein antigens. See, for example, Weiner H L, Immmunol Today, 18:335, 1997.

Immune responses to pathogens or tumors are currently altered by delivering proteins, polypeptides, or peptides, alone or in combination with adjuvants. For example, the hepatitis B virus vaccine contains recombinant hepatitis B virus surface antigen, a non-self antigen, formulated in aluminum hydroxide, which serves as an adjuvant. This vaccine induces an immune response against hepatitis B virus surface antigen to protect against infection. An alternative approach involves delivery of an attenuated, replication deficient, and/or non-pathogenic form of a virus or bacterium, each non-self antigens, to elicit a host protective immune response against the pathogen. For example, the oral polio vaccine is composed of a live attenuated virus, a non-self antigen, which infects cells and replicates in the vaccinated individual to induce effective immunity against polio virus, a foreign or non-self antigen, without causing clinical disease. Alternatively, the inactivated polio vaccine contains an inactivated or ‘killed’ virus that is incapable of infecting or replicating, and if administered subcutaneously, to induce protective immunity against polio virus.

### Mechnisms of Initiation and Propagation of Immune Responses

Inflammatory Diseases Associated With “Nonself Molecules”: Infection with microorganisms, including mycoplasma, viruses, bacteria, parasites and mycobacteria, leads to inflammation in target organs, and in some cases systemic inflammation. Prominent examples include bacterial septic arthritis, Lyme arthritis, infectious uveitis, and septic shock.

As part of the inate immune system, inflammatory mediators such as components of the clotting cascade, bradykinins, and complement are activated and contribute to inflammation and morbidity. The immune response in infectious disease is directed against non-self molecules present in the microorganisms, including proteins, lipids, carbohydrates, and nucleic acids. Bacterial DNA containing certain motifs referred to as “CpG” motifs, defined in more detail below, are capable of initiating inflammatory responses in animal models. For example, injection of bacterial DNA or CpG motifs, both of which are non-self molecules, into synovial joints mimics many of the inflammatory signs and symptoms that characterize septic arthritis.

Inflammatory Diseases Associated With “Self Molecules”: Many human diseases are associated with acute or chronic inflammation in the absence of any known infectious etiology. In these diseases, the immune system is active, causing the affected tissues to be inflamed and abnormally infiltrated by leukocytes and lymphocytes, but there appears to be no associated infection. Examples include osteoarthritis, coronary artery disease, Alzheimer's Disease, certain forms of dermatitis, gastritis, and pneumonitis. The predominant immune response is an innate immune response, in the absence of an adaptive immune response.

Autoimmune Diseases Associated With “Self Molecules”: Dozens of autoimmune diseases have been described, including rheumatoid arthritis, systemic lupus erythematosus, multiple sclerosis, diabetes mellitus, psoriasis, and many others. Like the inflammatory diseases associated with self molecules above, the immune system is active, causing the affected tissues to be inflamed and abnormally infiltrated by leukocytes and lymphocytes, and there appears to be no associated infection. Unlike the inflammatory diseases associated with self molecules, a defining characteristic of autoimmune diseases is the presence of autoantibodies and/or T cells specific for self molecules expressed by the host. The mechanisms by which self molecules are selectively targeted by the host T and B lymphocytes are obscure. Some investigators have suggested that autoimmune diseases are triggered or exacerbated by infections with microbial pathogens. Stimulation with microbial CpG sequences is associated with an increased susceptibility to the development of animal models of autoimmune diseases such as EAE (Segal et al., J. Immunology, 158:5087, 1997) and SLE (Gilkeson et al., J. immunology, 142: 1482, 1989); however, there is little evidence to support the hypothesis that CpG sequences or microbial products can themselves trigger an autoimmune disease in an otherwise healthy animal, although inflammatory diseases can be induced. For example, several important experiments using gnotobiotic systems (i.e., animals raised in a germ free environment) have demonstrated that spontaneous development of autoimmune diseases occurs without exposure to naturally occurring microbes or microbial CpGs. Examples include development of autoimmune skin and genital disease in a germfree transgenic rodent model of ankylosing spondylitis (Taurog, J Exp Med, 180:2359, 1994,); and development of lupus in 2 different models of SLE (Maldonadoi et al., J Immunol, 162: 6322, 1999; Unni et al., J Rheum, 2:35, 1975). An inducible model of SLE has also been described in which a single injection of any mouse strain with the hydrocarbon oil, pristane, leads to the development of SLE, characterized by the production of characteristic autoantibodies and immune complex-mediated kidney disease. Taken together, these experimental models suggest that spontaneous and inducible autoimmune diseases can develop in the absence of exposure to microbial DNA or CpGs.

Immunostimulatory sequences (ISS): The innate immune system is regarded as the first line of defense against microbes and pathogens. One of the most potent stimulants of the innate immune system is microbial DNA, which contains immunostimulatory sequences (ISS). The activation of innate immunity by specific immune stimulatory sequences in bacterial DNA requires a core unmethylated hexameric sequence motif consisting of 5′-purine-purine-cytosine-guanine-pyrimidine-pyrimidine-3′ for stimulation in mice and 5′-purine-pyrimidine-cytosine-guanine-pyrimidine-pyrimidine-3′ for stimulation in humans (Krieg et al., Annu Rev. Immunol., 20:709-760, 2002). Bacterial DNA and synthetic oligodeoxynucleotides (ODN) containing this dinucleotide motif, referred to as “CpG” sequences, within an immune stimulatory sequence motif have the ability to stimulate B cells to proliferate and secrete IL-6, IL-10, and immunoglobulin (Krieg et al., Nature, 374:546-549, 1995; Yi et al., J. Immunol., 157:5394-5402, 1996). ISS DNA also directly activates dendritic cells, macrophages and monocytes to secrete Th1-like cytokines such as TNF-α, IL6, and IL12 and up-regulates the expression of MHC and costimulatory molecules (Klinman et al., Proc. Nat. Acad. Sci. U.S.A., 93:2879-2883, 1996; Martin-Orozco et al., Int. Immunol., 11:1111-1118, 1999; Sparwasser et al., Eur. J. Immunol., 28:2045-2054, 1998). In mice, Toll-like receptor-9 (TLR-9) has been identified as the key receptor in the recognition of CpG motifs.

In vertebrate DNA, the frequency of CpG dinucleotides is suppressed to about one quarter of the predicted (expected) value, and the C in the CpG dinucleotide is methylated approximately 80% of the time. By contrast, bacterial DNA, like synthetic ODN, the C is not preferentially methylated in the CpG dinucleotide. Thus, bacterial DNA is structurally distinct from vertebrate DNA in its greater than 20-fold increased content of unmethylated CpG motifs. Numerous studies have established the unmethylated CpG motif as the molecular pattern within bacterial DNA that activates immune cells (Krieg et al., Annu. Rev. Immunol., 20:709-760, 2002).

CpG DNA is recognized as a potent adjuvant for its ability to induce a strong antibody response and Th1-like T-cell response to such nonself antigens as hen egg lysozyme and ovalbumin (Chu et al., J. Exp. Med., 186:1623-1631, 1997; Lipford et al., Eur. J. Immunol., 27:2340-2344, 1997). Currently, CpG DNA and CpG ODN are being utilized as therapeutic vaccines in various animal models of infectious diseases, tumors, allergic diseases, and autoimmune diseases (Krieg et al., Annu. Rev. Immunol., 20:709-760, 2002). The success of CpG as a vaccine apparently relies heavily on its effectiveness of inducing a strong Th1-like response, and in some instances, redirecting a Th2 response to a Th1 response, such as in the allergic asthma model (Kline et al., J. Immunol., 160:2555-2559, 1998; Broide et al., J. Immunol., 161:7054-7062, 1998).

There has been significant attention given to the therapeutic applications of innate immune activation by CpG DNA. The potent non-antigen specific innate immune cell activation induced by CpG DNA is sufficient to protect mice against bacterial challenge, and even to treat established infections with intracellular pathogens (Agrawal et al., Trends Mol. Med., 8:114-121, 2002). CpG DNA also induces innate immune resistance to tumors and the regression of established tumors in mice (Dow et al., J. Immunol., 163:1552-1561, 1999; Carpenter et al., Cancer Res., 59:5429-5432, 1999; Smith et al., J. Natl. Cancer Inst., 90:1146-1154, 1998). The potent Th1 adjuvant effect of CpG DNA can even override preexisting Th2 immune responses; it has been used as an adjuvant for allergy vaccines, where it induces Th1 responses to antigens in the presence of a preexisting Th2 response, leading to decreased symptoms following subsequent allergen inhalation (Van Uden et al., J. Allergy Clin. Immunol., 104:902-910, 1999).

Immunoinhibitory sequences (IIS): Inhibitors of immunostimulatory sequence oligodeoxynucleotide (ISS-ODN) have been used to inhibit the immunostimulatory activity of ISS-ODN, for example, to suppress the immunostimulatory activity of any ISS-ODN present in recombinant expression vectors particularly in the context of gene therapy, as anti-inflammatory agents for reducing host immune responses to ISS-ODN in bacteria and viruses, as autoimmune modulator in combination with autoantigen or autoantibody conjugate to inhibit ISS-ODN stimulated Th1 mediated IL-12 production, for use as an adjuvant for Th2 immune responses to extracellular antigen, and generally to shift a host immune response from a Th1 to a Th2 response. See e.g., WO 04/047734 and U.S. Pat. No. 6,255,292.

Yamada et al, J. Immunol., 169; 5590-5594, 2002, using various in vitro immune activation cell systems evaluated IIS oligodeoxynucleotides in CpG induced immune stimulation. Yamada et al. found that suppression by IIS oligodeoxynucleotides is dominant over stimulation by oligodeoxynucleotides and it is specific for CpG-induced immune responses. They found that the most suppressive oligonucleotide sequences contained polyG or G-C rich sequences, but a specific hexamer motif was not discovered. Krieg et al., PNAS, 95; 12631-12636, 1998, found that synthetic oligonucleotides containing neutralizing motifs defined by him as CpG dinucleotide in direct repeat clusters or with a C on the 5′ side or a G on the 3′ side, could block immune activation by immunostimulatory CpG motifs. Again, a hexamer immunoinhibitory squence was not discovered. In Zeuner et al., Arthritis and Rheumatism, 46: 2219-2224, 2002, the IIS described by Kreig at al. above, was demonstrated to reduce CpG induced arthritis in an animal model. Additional IIS have been described in: US 20050239732, Jurk et al. characterized by a CC dinucleotide 5′ of a G-rich oligomer and in Lenert et al., (2003, DNA Cell Biol. 22: 621-31) characterized by proximal pyrimidine-rich CCT sequence three to five nucleotides 5′ to a distal GGG triplet. However, a hexamer immunoinhibitory sequence was not discovered in either. In U.S. Pat. No. 6,225,292, Raz et al. describe a specific hexamer motif designated as 5′-purine-purine-[Y]-[Z]-pyrimidine-pyrimidine-3′ where Y is any nucleotide except cytosine, and Z is any nucleotide, wherein when Y is not guanosine or inosine, Z is guanosine or inosine, which blocks the stimulatory activity of CpG immunostimulatory sequences. In each of the above examples, the IIS was demonstrated to specifically inhibit immune activation caused by stimulatory CpG sequences.

### Nucleic Acid Therapy

Antisense Therapy: Antisense oligonucleotides were originally designed as complementary to specific target genes to decrease their expression (Krieg, Annu. Rev. Immunol., 20:709-760, 2002). In order to prevent the degredation of these olignucleotides the backbones were generally modified, such as to a phosphorothioate backbone. Although in many cases the antisense oligonucleotides did suppress the expression of target genes in tissue culture cells, in vivo experiments were less successful at altering expression. Instead, many investigators found unexpectedly that some of these oligonucleotides stimulated the immune response in vivo. For example, antisense oligonucleotide against the rev gene of the human immunodeficiency virus (HIV) had an immunostimulatory effect as manifested by increased B cell proliferation and splenomegaly (Branda et al., Biochem. Pharmacol., 45:2037-2043, 1993). Although no immediate immunostimulatory sequence motif was identified from these early studies, these findings led to the eventual search for specific immunostimulatory motifs.

Gene Therapy: Polynucleotide therapeutics, including naked DNA encoding peptides and/or polypeptides, DNA formulated in precipitation- and transfection-facilitating agents, and viral vectors have been used for “gene therapy.” Gene therapy is the delivery of a polynucleotide to provide expression of a protein or peptide, to replace a defective or absent protein or peptide in the host and/or to augment a desired physiologic function. Gene therapy includes methods that result in the integration of DNA into the genome of an individual for therapeutic purposes. Examples of gene therapy include the delivery of DNA encoding clotting factors for hemophilia, adenine deaminase for severe combined immunodeficiency, low-density lipoprotein receptor for familial hypercholesterolemia, glucocerebrosidase for Gaucher's disease, al-antitrypsin for al-antitrypsin deficiency, alpha- or Beta-globin genes for hemoglobinopathies, and chloride channels for cystic fibrosis (Verma and Somia, Nature, 389, 239-42, 1997).

DNA immunization to treat infection: In DNA immunization a non-replicating transcription unit can provide the template for the synthesis of proteins or protein segments that induce or provide specific immune responses in the host. Injection of naked DNA promotes vaccination against a variety of microbes and tumors (Robinson and Tones, Semin Immunol, 9, 271-83., 1997). DNA vaccines encoding specific proteins, present in viruses (hepatitis B virus, human immunodeficiency virus, rotavirus, and influenza virus), bacteria (mycobacterium tuberculosis), and parasites (malaria), all non-self antigens, are being developed to prevent and treat these infections (Le et al., Vaccine, 18, 1893-901, 2000; Robinson and Pertmer, Adv Virus Res, 55, 1-74, 2000).

DNA to treat neoplasia: DNA vaccines encoding major histocompatibility antigen class I, cytokines (IL-2, IL-12 and IFN-gamma), and tumor antigens are being developed to treat neoplasia (Wlazlo and Ertl, Arch Immunol Ther Exp, 49:1-11, 2001). For example, viral DNA encoding the B cell immunoglobulin idiotype (antigen binding region) has been administered to eliminate and protect against B cell-lymphomas (Timmerman et al., Blood, 97:1370-1377, 2001).

DNA immunization to treat autoimmune disease: Others have described DNA therapies encoding immune molecules to treat autoimmune diseases. Such DNA therapies include DNA encoding the antigen-binding regions of the T cell receptor to alter levels of autoreactive T cells driving the autoimmune response (Waisman et al., Nat Med, 2:899-905, 1996; U.S. Pat. No. 5,939,400). DNA encoding autoantigens were attached to particles and delivered by gene gun to the skin to prevent multiple sclerosis and collagen induced arthritis. (PCT Publ. No. WO 97/46253; Ramshaw et al., Immunol., and Cell Bio., 75:409-413, 1997) DNA encoding adhesion molecules, cytokines (TNF alpha), chemokines (C-C chemokines), and other immune molecules (Fas-ligand) have been used to treat animal models of autoimmune disease (Youssef et al., J Clin Invest, 106:361-371, 2000; Wildbaum et al., J Clin Invest, 106:671-679, 2000; Wildbaum et al., J Immunol, 165:5860-5866, 2000; Wildbaum et al., J Immunol, 161:6368-7634, 1998; and Youssef et al., J Autoimmun, 13:21-9, 1999).

It is an object of the present invention to provide a method and composition for treating or preventing a disease, particularly autoimmune disease or inflammatory disease, comprising the administration of immune modulatory nucleic acids. Another object of this invention is to provide the means of identification of the immune modulatory sequences for treating disease. Yet another object of this invention is to provide the method and means of treating a disease associated with self-antigen(s), -protein(s), -polypeptide(s), or -peptide(s) that are present and involved in a non-physiological process in an animal comprising the administration of an immune modulatory sequence in combination with a polynucleotide encoding self-antigen(s), -proteins(s), -polypeptide(s) or -peptide(s). Another object of the present invention is to provide a composition for treating or preventing a disease associated with self-antigen(s), -proteins(s), -polypeptide(s), or -peptide(s) that is present non-physiologically in an animal. The invention further relates to the treatment or prevention of disease comprising the administration of the immune modulatory nucleic acids in combination with self-molecule(s). These and other objects of this invention will be apparent from the specification as a whole.

### BRIEF SUMMARY OF THE INVENTION

The present invention is based on the discovery of improved immune modulatory sequences that alone or in combination can be used to prevent or treat autoimmune or inflammatory diseases associated with self-molecules.

In particular, the present invention provides an improved immune modulatory sequence (IMS) comprising:

1.) a hexameric sequence
5′-Purine-Pyrimidine_{[1]}-[X]-[Y]-Pyrimidine_{[2]}-Pyrimidine_{[3]}-3;
wherein X and Y are any naturally occurring or synthetic nucleotide, except that
a. X and Y cannot be cytosine-guanine;
b. X and Y cannot be cytosine-cytosine when Pyrimidine_{[2]} is thymine
c. X and Y cannot be cytosine-thymine when Pyrimidine_{[1]} is cytosine

2.) a CC dinucleotide 5′ to the hexameric sequence wherein the CC dinucleotide is positioned between one to five nucleotides 5′ of the hexameric sequence; and

3.) a polyG region 3′ of the hexameric sequence wherein the polyG comprises at least three contiguous Gs and is positioned between two to five nucleotides 3′ of the hexameric sequence;

wherein the immune modulatory sequence does not contain cytosine-guanine sequences.

Alternatively, the present invention provides an improved immune modulatory sequence comprising:

1.) a hexameric sequence
5′-Purine-Pyrimidine-[X]-[Y]-Pyrimidine-Pyrimidine-3′;
wherein X and Y are guanine-guanine;

2.) a CC dinucleotide 5′ to the hexameric sequence wherein the CC dinucleotide is positioned between one to five nucleotides 5′ of the hexameric sequence; and

3.) a polyG region 3′ of the hexameric sequence wherein the polyG comprises between two and ten contiguous Gs and is positioned between two to ten nucleotides 3′ of the hexameric sequence;

wherein the immune modulatory sequence does not contain cytosine-guanine sequences.

In some embodiments of the present invention, X and Y of the hexameric sequence are GpG. In certain embodiments the hexameric sequence is 5′-GTGGTT-3′. In some embodiments the CC di-nucleotide is positioned two nucleotides 5′ of the hexameric sequence. In certain embodiments the polyG region comprises three contiguous guanine bases and is positioned two nucleotides 3′ from the hexameric sequence. In certain embodiments the improved immune modulatory sequence is 5′-CCATGTGGTTATGGGT-3′.

Objects of the present invention are accomplished by a novel method and composition to treat or prevent a disease, particularly an autoimmune or inflammatory disease, comprising the administration of immune modulatory nucleic acids having one or more immune modulatory sequences. The immune modulatory nucleic acids can be administered alone or in combination with a polynucleotide encoding self-antigen(s), -protein(s), -polypeptide(s), -peptide(s). The immune modulatory nucleic acids may also be administered in combination with other self molecules to treat an autoimmune or inflammatory disease associated with one or more self-molecules that is present in the individual nonphysiologically.

The invention further relates to pharmaceutical compositions for the treatment or prevention of an autoimmune or inflammatory disease wherein the pharmaceutical composition comprises an immune modulatory sequence in the form of a polynucleotide, such as a DNA polynucleotide. The immune modulatory sequence may also be embodied within a vector, by modification of elements of a vector nucleotide sequence to include immune modulatory sequence motifs further comprising an inhibitory dinucleotide motif when used in the context of diseases associated with self-molecules present in the subject non-physiologically, such as in autoimmune or inflammatory disease.

Other objects of the present invention are accomplished by a novel method of treating or preventing a disease in an animal associated with one or more self-antigen(s), -protein(s), -polypeptide(s), or -peptide(s) that is present in the animal nonphysiologically comprising administering to the animal an immune modulatory sequence. The invention further relates to a novel method of treating or preventing a disease in an animal associated with one or more self-antigen(s), -protein(s), -polypeptide(s), or -peptide(s) that is present in the animal nonphysiologically comprising administering to the animal an immune modulatory sequence in combination with a polynucleotide encoding the self-antigen(s), -protein(s), -polypeptide(s) or -peptide(s).

In one aspect of the invention there is provided a method for treating or preventing autoimmune diseases such as multiple sclerosis, rheumatoid arthritis, insulin dependent diabetes mellitus, autoimmune uveitis, primary biliary cirrhosis, myasthenia gravis, Sjogren's syndrome, pemphigus vulgaris, scleroderma, pernicious anemia, systemic lupus erythematosus (SLE), ankylosing spondylitis, autoimmune skin diseases, and Grave's disease comprising administering to the animal an immune modulatory sequence either alone or in combination with a self-vector comprising a polynucleotide encoding a self-antigen(s), -protein(s), -polypeptide(s) or -peptide(s) associated with the autoimmune disease. In another aspect of the invention the immune modulatory sequence is administered in combination with a polynucleotide comprising DNA encoding the self-antigen(s), -proteins(s), -polypeptide(s), or -peptide(s) present in the subject in a non-physiological state and associated with a disease.

In another aspect of the invention there is provided a method for treating or preventing inflammatory diseases such as osteoarthritis, gout, pseudogout, hydroxyapatite deposition disease, asthma, bursitis, tendonitis, conjunctivitis, urethritis, cystitis, balanitis, dermatitis, coronary artery disease, or migraine headache comprising administering to the animal an immune modulatory sequence, either alone or in combination.

In yet another aspect of the invention there is provided a method for treating or preventing diseases related to organ or cell transplantation including but not limited to GVHD or transplant rejection comprising administering to the animal an immune modulatory sequence, either alone or in combination with a self-vector comprising a polynucleotide encoding a self-antigen(s), -proteins(s), -polypeptide(s) or -peptide(s) associated with GVHD or transplant rejection. Administration of the immune modulatory sequence and the self-vector comprising a polynucleotide encoding the self-antigen(s), -proteins(s), -polypeptide(s), or -peptide(s) modulates an immune response to the self-antigen(s), -proteins(s), -polypeptide(s) or -peptide(s) expressed by the self-vector.

In some embodiments of the methods and compositions, a plurality of (i.e., two or more) immune modulatory sequences are used, separately or linked together, e.g., in succession or in tandem. The two or more IMS can be the same or different.

### BRIEF DESCRIPTION OF THE DRAWINGS

FIG. 1: Inhibitory IMS suppress CpG dependent proliferation of human PBMC cells. Human PBMCs (5×105/ml) were incubated in the presence of stimulatory CpG-ODN (5 μg/ml), or a mixture of CpG and inhibitory IMS. Cells were incubated with DNA for 96 hrs and wells were pulsed with 1 μCi[³H]TdR for the final 24 hrs of culture before incorporated radioactivity was measured. Each data point represents the mean of 4 replicates. a,b) The stimulatory CpG-ODN 2395 (5 μg/ml) was incubated independently (second bar from left) or with increasing concentrations of inhibitory IMS (1-25 μg/ml as indicated in parentheses+5 μg/ml 2395) in two different cell donors−QB8 (a) and QB 10 (b).

FIG. 2: Dose response analysis of the IMS GpG.1 and I18 effects on CpG stimulated cytokine production. Human PBMCs (5×10⁶/ml) were incubated for 48 hrs in the presence of CpG ODN (2006, 2395, C274, D19) alone or in combination with increasing doses of the IMS GpG.1 and I18 (all IMS samples contained 5 μg/ml of the CpG oligo). Cytokine levels in the media were measured by ELISA. Each data point represents the average of three replicates. For IL-10 and IL-12 (a & b) there is increased suppression of cytokine production with increased IMS dose. For IFN-gamma (c) and IFN-alpha (d) increasing IMS dose causes increased cytokine expression for both IMS although for IMS I18 the low dose suppresses the overall IFN-gamma levels and all I18 doses suppress IFN-alpha levels relative to the CpG alone sample.

FIG. 3: ConA and PoIyI:C inhibitory effects of IMS GpG.1 and I18 a) Human PBMCs (5×10⁶/ml) were incubated with Poly I:C (10 μg/ml) alone or with increasing concentrations of IMS for 48 hrs. Supernatant IFN-alpha protein levels were measured by ELISA. Each data point represents the average of three replicates. The 5 μg (25 μg/ml) doses of I18 and GpG.1 were effective at suppressing Poly I:C induced IFN-alpha. b) PBMCs were incubated with 10 μg/ml of ConA alone or in combination with GpG.1 and I18 (25 μg/ml each) and proliferation was analyzed as described in FIG. 1.

FIG. 4: Inhibitory IMS can induce cytokine production independent of CpG. Increasing doses of IMS in the absence of CpG oligo were incubated with PBMCs (donors QB11 and QB12) for 48 hrs and cytokines were analyzed by ELISA. Each data point represents the average of three replicates. a) IL-6 b) IL-10 c) IFN-alpha d) IFN-gamma.

FIG. 5: Inhibitory IMS can stimulate PBMC proliferation in the absence of stimulatory CpG ODN. Human PBMCs (5×10⁵/ml) were incubated in the presence of the stimulatory CpG-ODN 2395 (5 μg/ml) or increasing concentrations of the IMS GpG.1 and I18. Cell proliferation was measured as described above (FIG. 1).

FIG. 6: Inhibitory IMS can suppress CpG induced IL-12 expression in vivo. Oligonucleotides were administered by intraperitoneal injection and 24 hrs later serum was drawn by retro-orbital bleeding. Serum was analyzed for IL-12 levels by ELISA.

FIG. 7: Weekly IMS oligo dosing at 50 μg does not significantly affect progression to proteinurea in a mouse model of lupus. NZB/W F1 female mice treated with TpT or GpG oligo and control groups treated with PBS were scored weekly for presence of protein in the urine. The percentage of mice displaying proteinurea, defined as 2 consecutive scores of >300 mg/dl as scored by Albustix Reagent Strips, were plotted over time. No significant delay in onset of proteinurea was observed in any treatment group.

FIG. 8: Weekly IMS oligo dosing at 50 μg does not significantly affect anti-DNA autoantibody titer in mouse model of lupus. Sera from NZB/W F1 female mice treated with TpT or GpG oligo and control groups treated with PBS was harvested at the time of sacrifice. Anti-double stranded DNA antibody titer was measured using a commercially available kit. Treatment with oligos slightly lowers the overall anti-DNA response, but none reached statitistical significance.

FIG. 9: GpG IMS oligo administered by oral gavage significantly decreased severity of inflammation in kidneys in a mouse model of lupus. Histopathology was scored on kidneys taken from NZB/W F1 female mice treated with TpT or GpG oligo and control groups treated with PBS that had progressed to proteinurea. The scoring system was designed to measure the extent of inflammation and was defined as: 1=minimal; 2=mild; 3=moderate; and 4=marked/severe. Scoring was performed blindly by a contract veterinarian pathologist. Histology scores were averaged for each group and are shown below as the average ±SEM. A reduction in kidney inflammation was observed with both GpG treated groups, however only the GpG administered by oral gavage reached statistical significance.

FIG. 10: Dose dependent delay in proteinurea onset with GpG IMS oligo treatment in a mouse model of lupus. NZB/W F1 female mice treated with increasing dosages (50, 200 and 500 μg) of the GpG oligo by IP weekly and control animals treated with PBS vehicle were scored weekly for presence of protein in the urine. The percentage of mice displaying proteinurea, defined as 2 consecutive scores of >300 mg/dl as scored by Albustix Reagent Strips, were plotted over time. There was a dose dependent delay in proteinurea onset with the highest dose of GpG providing the most significant delay (p=0.03).

FIG. 11: Dose dependent decrease in anti-DNA antibody response with GpG IMS oligo treatment in a mouse model of lupus. Sera from NZB/W F1 female mice treated with increasing dosages (50, 200 and 500 μg) of GpG oligo by IP or ID weekly and control animals treated with PBS vehicle was harvested at the time of sacrifice. Anti-double stranded DNA antibodies were measured using a commercially available kit. A plot of antibody titer reveals a dose dependent decrease in anti-DNA response with increasing GpG concentrations.

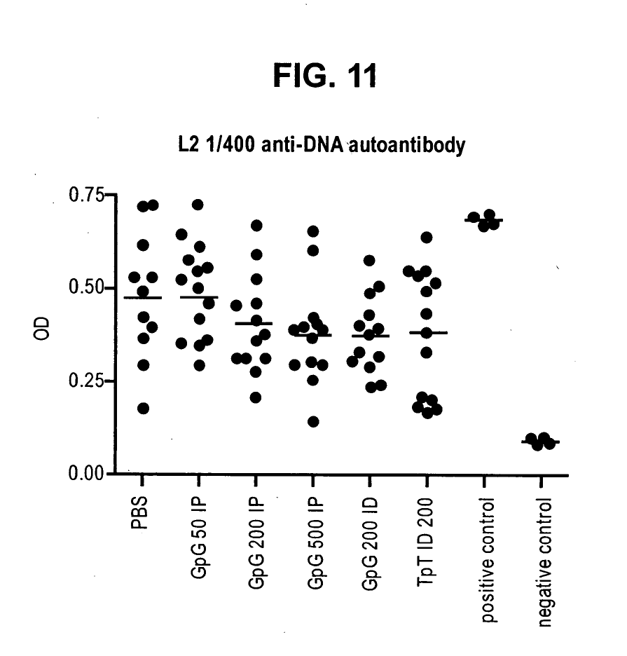

FIG. 12: I-18m IMS oligo treatment significantly lowers anti-DNA antibody response in a mouse model of lupus. Sera from NZB/W F1 female mice treated with 50 μg of GpG, I-18h, I18m or TpT daily by IP injection and control group treated with PBS vehicle alone was collected at the time of sacrifice. Anti-double stranded DNA antibodies were measured using a commercially available kit. A plot of antibody titer reveals that I-18m treatment significantly lowered autoantibody levels to DNA compared to control.

FIG. 13: GpG IMS oligo in combination with low dose steroid decreases inflammation associated with EAU. B10.RIII mice immunized with hIRBP₁₆₁₋₁₈₀ peptide were dosed ID weekly with 200 μg GpG or TpT plus low dose Depromedrol (1 mg/kg). Histological evaluation of eyes at day 21 was scored blindly by an expert in EAU to give an average severity score for each experimental group. Although administration of steroid alone or steroid plus TpT IMS oligo has no significant affect on the severity of uveitis, treatment with steroid plus GpG significantly lowered disease scores.

FIG. 14: GpG IMS oligo treatment alone significantly lowers severity of inflammation in EAU. B10.RIII mice immunized with hIRBP₁₆₁₋₁₈₀ peptide were administered 200 μg GpG or TpT oligos alone or in combination with low dose Depromedrol (1 mg/kg) intraperitoneal or intradermal were sacrificed and eyes were harvested for histological evaluation. Eyes were scored blindly by an expert in EAU. While no significant effect of the steroid alone or in combination with GpG oligo on the severity of uveitis was observed, IP delivery of GpG alone provided significant improvement in severity scores similar to the anti-CD3 positive control.

FIG. 15: Daily IP delivery of IMS oligos does not affect EAU disease severity. B10.RIII mice immunized with hIRBP₁₆₁₋₁₈₀ peptide were dosed daily with I-18h, I-18m, GpG or TpT by IP injections beginning on day 0. At day 21, animals were sacrificed and the eyes harvested for histology. Eye histology was scored blindly by an expert in EAU. IMS oligos had no significant effect on EAU disease severity.

FIG. 16: Treatment with GpG IMS oligos lowers EAU disease severity scores after adoptive transfer. Lymph node and spleen cells from hIRBP₁₆₁₋₁₈₀ immunized mice were cultured in vitro for three days with inducing peptide. On day four, 3×10⁷ cells were transferred into naïve B10.RIII animals who were then treated weekly with 200 μg of GpG oligo or PBS by IP delivery. A trend towards lowering disease severity was observed.

FIG. 17: I-18h IMS oligo significantly decreases mean arthritis incidence in a collagen antibody induced arthritis model. Balb/c mice injected IV with monoclonal anti-collagen arthritogenic antibodies on day 0 were treated on days 4-10 with 50 μg IMS oligo administered daily by IP injection. Animals were observed and disease scored daily. Mean arthritis scores for each experimental group are shown over time. Treatment with I-18h oligo significantly reduced the mean arthritis score compared to both the PBS control group and treatment with GpG oligos.

FIG. 18: I-18h significantly decreases incidence of arthritis in the collagen antibody induced arthritis model. Balb/c mice injected IV with monoclonal anti-collagen arthritogenic antibodies on day 0 were treated on days 4-10 with 50 μg IMS oligo administered daily by IP injection. Animals were observed and disease scored daily. Treatment with I-18h oligos significantly reduced the arthritis incidence compared to both the PBS control group and treatment with GpG oligos.

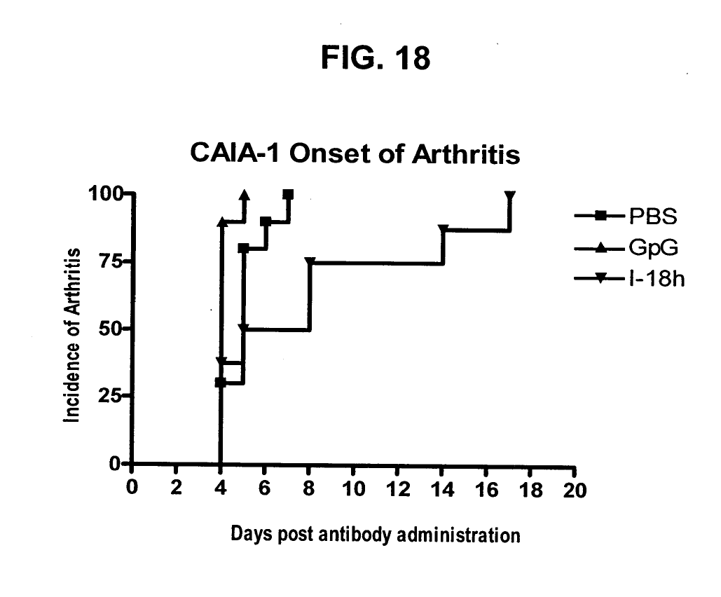

FIG. 19: Pre-treatment with GpG oligos decreases subsequent weight loss in response to TNBS induced colitis. C3H mice treated rectally with a sub-colitogenic dose of TNBS (0.5%) on day −5 were administered GpG oligos daily from day −5 through day 0 when a colitogenic dose of TNBS was administered (3.5%). Mean weight loss and standard error (SEM) of each group was calculated and graphed. Untreated controls are animals that were not given TNBS. Vehicle controls were treated with TNBS and treated with PBS on the same schedule as oligo treatment. Statistical analysis revealed that treatment with either 10 or 100 μg doses of GpG oligo were significantly better than the vehicle treated control group, whereas the GpG oligo 50 μg dose group did not reach statistical significance.

FIG. 20: Pre-treatment with I-18h oligos decreases subsequent weight loss in response to TNBS induced colitis. C3H mice treated rectally with a sub-colitogenic dose of TNBS (0.5%) on day −5 were administered I-18h oligos daily from day −5 through day 0 when a colitogenic dose of TNBS was administered (3.5%). Mean weight loss and standard error (SEM) of each group was calculated and graphed. Untreated controls are animals that were not given TNBS. Vehicle controls were treated with TNBS and treated with PBS on the same schedule as oligo treatment. Statistical analysis revealed that treatment with I-18h oligos at all dosages were significantly better than the vehicle treated control group.

FIG. 21: Pre-treatment with I-18m oligos decreases subsequent weight loss in response to TNBS induced colitis. C3H mice treated rectally with a sub-colitogenic dose of TNBS (0.5%) on day −5 were administered I-18h oligos daily from day −5 through day 0 when a colitogenic dose of TNBS was administered (3.5%). Mean weight loss and standard error (SEM) of each group was calculated and graphed. Untreated controls are animals that were not given TNBS. Vehicle controls were treated with TNBS and treated with PBS on the same schedule as oligo treatment. Statistical analysis revealed that treatment with 50 μg of I-18m oligo was significantly better than the vehicle treated control group, whereas the 100 μg dose level did not reach statistical significance.

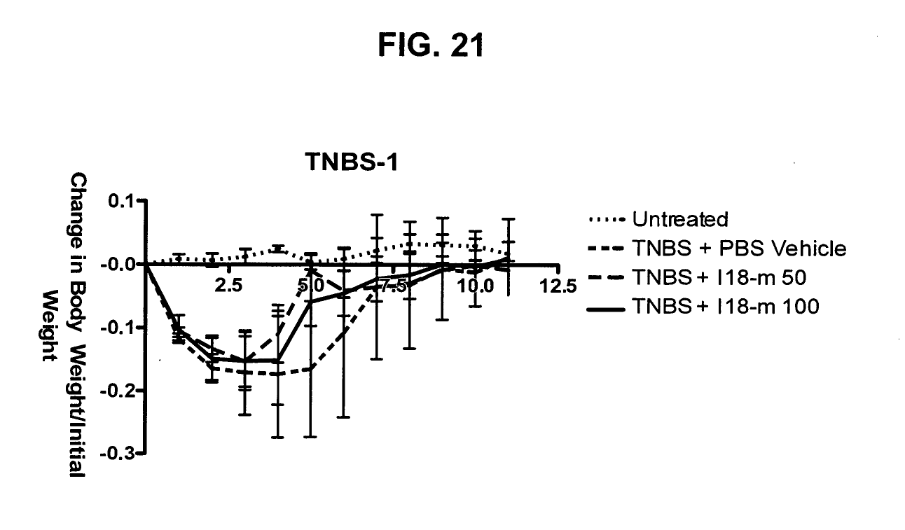

FIG. 22: Pretreatment with GpG significantly decreases weight loss associated with DSS induced colitis. Female C3H mice pretreated beginning at day −2 with IP injections of 50 or 200 μg of GpG oligo were fed 3.5% DSS in drinking water from day 0-7 to induce acute colitis. Mean weight loss and standard error (SEM) of each group was calculated and graphed. Untreated controls are animals that were not given DSS. The vehicle control group was treated with DSS and given PBS on the same schedule as oligo treatment. Statistical analysis revealed a significant decrease in weight loss in the 50 μg GpG oligo treated group compared to the vehicle treated control group (p<0.05; one way ANOVA with Dunnett's Multiple Comparison). The 200 μg dose level did not reach statistical significance (p>0.05).

FIG. 23: Pretreatment with I-18h oligo significantly decreases weight loss associated with DSS induced colitis. Female C3H mice pretreated beginning at day −2 with IP injections of 50 or 200 μg of I-18h oligo were fed 3.5% DSS in drinking water from day 0-7 to induce acute colitis. Mean weight loss and standard error (SEM) of each group was calculated and graphed. Untreated controls are animals that were not given DSS. The vehicle control group was treated with DSS and given PBS on the same schedule as oligo treatment. Statistical analysis revealed a significant decrease in weight loss in the 50 μg I-18h treated group compared to the vehicle treated control group (p<0.05; one way ANOVA with Dunnett's Multiple Comparison). The 200 μg dose level did not reach statistical significance (p>0.05)

FIG. 24: Treatment with GpG oligos beginning at time of disease induction significantly decreases weight loss associated with DSS induced colitis. Female C3H mice treated at day 0 with IP injections of GpG oligos were fed 3.5% DSS in drinking water from day 0-7 to induce acute colitis. Mean weight loss and standard error (SEM) of each group was calculated and graphed. Untreated controls are animals that were not given DSS. The vehicle control group was treated with DSS and given PBS on the same schedule as oligo treatment. Statistical analysis revealed a significant decrease in weight loss in the 50 μg (p<0.01) and 200 μg (p<0.05) GpG oligo treated groups compared to the vehicle treated control group (one-way ANOVA with Dunnett's Multiple Comparison). Furthermore, the 50 μg GpG oligo treated group was not signficantly different (p>0.05) from the untreated (no DSS) control group suggesting a complete blocking of DSS induced colitis at this dose level of GpG oligo.

FIG. 25: Treatment with I-18h oligos beginning at time of disease induction has no significant effect on weight loss associated with DSS induced colitis. Female C3H mice treated at day 0 with IP injections of I-18h oligos were fed 3.5% DSS in drinking water from day 0-7 to induce acute colitis. Mean weight loss and standard error (SEM) of each group was calculated and graphed. Untreated controls are animals that were not given DSS. The vehicle control group was treated with DSS and given PBS on the same schedule as oligo treatment. Statistical analysis revealed no significant decrease in weight loss in either the 50 μg or 200 μg I-18h oligo treated groups compared to the vehicle treated control group (one-way ANOVA with Dunnett's Multiple Comparison).

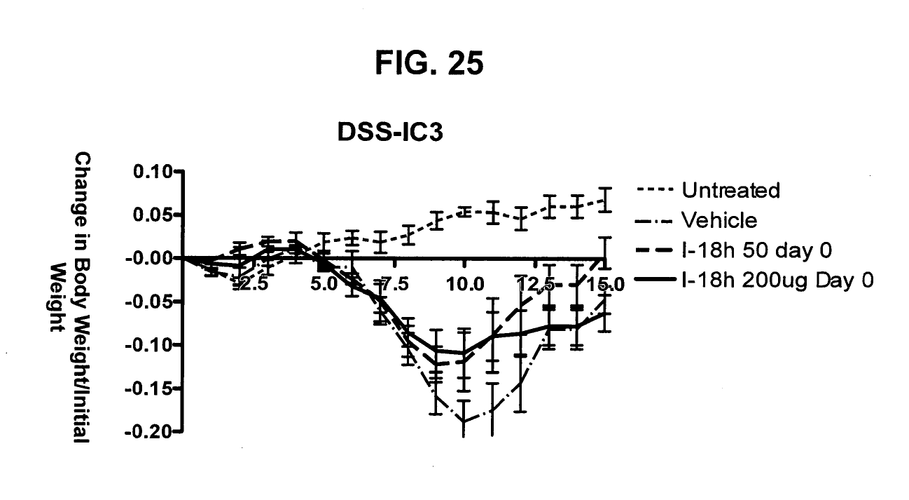

FIG. 26: I18 Mutagenesis. Human PBMCs were incubated in the presence of stimulatory CpG-ODN (5 μg/ml) and inhibitory IMS derived from I18. Cells were incubated with DNA for 96 hrs and wells were pulsed with 1 μCi[³H]TdR for the final 24 hrs of culture before incorporated radioactivity was measured. I18 derived sequences are shown (above) with the percentage inhibition of CpG stimulated proliferation (below). Mutations within the polyG region (I18.M3-6 & 8) significantly reduced the ability of oligonucleotides containing the hexameric sequence 5′-GTGGTT-3′ to inhibit PBMC proliferation from two different donors.

FIG. 27: I18 Mutagenesis. Human PBMCs were incubated in the presence of stimulatory CpG-ODN (5 μg/ml) and inhibitory IMS derived from I18. Cells were incubated with DNA for 96 hrs and wells were pulsed with 1 μCi[³H]TdR for the final 24 hrs of culture before incorporated radioactivity was measured. I18 derived sequences are shown (above) with the percentage inhibition of CpG stimulated proliferation (below). Mutations 5′ to the hexameric sequence (I18.M10-12) significantly reduced the ability of oligonucleotides containing the hexameric sequence 5′-GTGGTT-3′ to inhibit PBMC proliferation. Furthermore, addition of nucleotides between the hexameric sequence and the polyG modestly reduced PBMC proliferation (I 18.M13-16).

FIG. 28 illustrates a comparison of the nucleic acid sequences of human I18 and mouse I18.

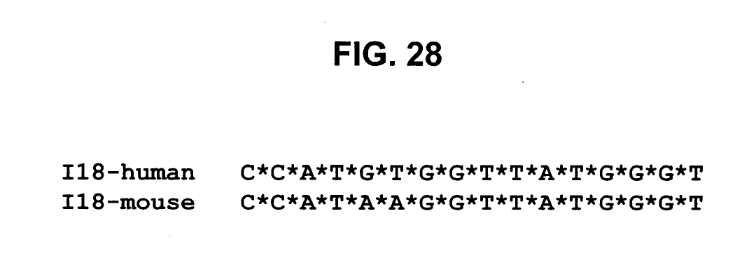

FIG. 29: I18 Inhibits TLR3, 5, 7 and 9. HEK 293 cells expressing TLR2, 3, 4, 5, 7, 8 or 9 were incubated with immune modulatory sequences including I18 at 25 μg/mL in the presence of the corresponding TLR ligand, and activation of NF-κB was determined. Baseline signaling in the absence of ligand is shown in the first row (No Ligand), whereas activation of TLRs by their corresponding ligands is shown in the final row (Control +). I18 in the presence of ligand inhibits signaling by TLR3, 5, 7 and 9 (I18+Ligand; second row from front).

FIG. 30: I18 Inhibits TLR7 Ligand Induced Production of IFN-alpha by pDCs. A. pDCs isolated from Donor 1 produce IFN-alpha when incubated with TLR7 ligand loxoribine or R-837. IFN-alpha production is completely blocked by I18 at 5 μg/mL or 25 μg/mL. B. Similarly, I18 at 5 μg/mL completely blocks IFN-alpha expression by pDCs isolated from Donor 2 in response to TLR7 ligand (loxoribine versus lox+I18).

FIG. 31: I18 Inhibits TLR3 Ligand Induced Production of IFN-alpha by PBMC. A. PBMC isolated from Donor 1 produce IFN-alpha in response to PolyI:C, and this is blocked by I18 at 25 μg/mL. B. Production of IFN-alpha by TLR3 activation in PBMC isolated from Donor 2 is blocked by both 5 μg/mL and 25 μg/mL I18.

FIG. 32: I18 Suppresses CpG Induced Production of IFN-alpha by pDC. A, B. IFN-alpha production by pDCs isolated from Donor 1 and 2 incubated with immune stimulatory CpG sequences alone (CpG) or in the presence of increasing amounts of I18 (CpG+I18) was measured by ELISA. I18 suppresses IFN-alpha production. C, D. IFN-alpha production by pDCs isolated from Donor 1 and 2 incubate with CpG sequences alone (C274) or after pre-incubated with I18 for 24 hours at equal molar ratios (I18(1)(To)+C274(1)(24 hrs)) or with 5 fold excess of I18 (I18(5)(To)+C274(1)(24 hrs). Pre-incubation with I18 completely blocks IFN-alpha production.

FIG. 33: Immune Complexes from SLE Patients with Anti-dsDNA Antibodies Induce Production of IFN-alpha by pDCs. A. Serum from four SLE patients (SLE 19558; SLE 22914; SLE KP491; SLE KP504) versus a normal control (Normal) was assayed for anti-dsDNA antibodies by ELISA. B. Serum immune complexes were isolated from four SLE patients (SLE 19558; SLE 22914; SLE KP491; SLE KP504) and a normal control (Normal). C. Isolated immune complexes were incubated with isolated human pDC and production of IFN-alpha was assayed by ELISA. pDCs alone (Cells only) produce little IFN-alpha but are induced by immune stimulatory CpG sequences and immune complexes from SLE patients with anti-dsDNA antibodies (19558 and 22914). In contrast, immune complexes from SLE patients without anti-dsDNA antibodies (KP491 and KP504) or a normal control (Normal SG) do not induce IFN-alpha production.

FIG. 34: I18 Inhibits SLE-Immune Complex Induction of IFN-alpha by pDCs. Purified Ig from SLE patients whose serum contains anti-dsDNA antibodies and a normal control were incubated for 24 hours with isolated pDCs with or without I18. Isolated pDCs (Cells only) or pDCs incubated with immune complexes from a normal control (Normal) produced little IFN-alpha. In contrast, pDCs incubated with immune complexes from SLE patients produced significant amounts of IFN-alpha (SLE 19558; SLE 22914). Production of IFN-alpha is inhibited by I18 (SLE 19558+I18; SLE 22914+I18).

FIG. 35: I18 Inhibits CpG Activation of Normal Peripheral CD19+ B Cells. A, B. CD19+ B cells were incubated alone (No DNA), with 5 μg/mL stimulatory CpG-ODN (CpG(5)), or with 5 μg/mL stimulatory CpG-ODN in the presence of 5 μg/mL I18 (CpG+I18(5)), and cytokine levels were analyzed by ELISA. I18 suppressed both CpG stimulated IL-6 (A) and IL-10 (B) expression. C. CD19+ B cells were incubated alone (No DNA), with 5 μg/mL stimulatory CpG-ODN (CpG), with 5 μg/mL stimulatory CpG-ODN in the presence of 5 μg/mL I18 (CpG+I18(5)), or with 5 μg/mL stimulatory CpG-ODN in the presence of 25 μg/mL I18 (CpG+I18(25)). Cell proliferation was assayed by [³H] thymidine incorporation. I18 significantly suppressed CpG stimulated B cell proliferation at both dosages.

FIG. 36: I18 Inhibits CpG Activation of Peripheral CD19+ B Cells from a Patient Diagnosed with SLE. A, B. CD19+ B cells were incubating alone (Cells only), with 5 μg/mL stimulatory CpG (CpG(5)), with 5 μg/mL stimulatory CpG in the presence of 5 μg/mL (CpG+I18(5)), or with 5 μg/mL stimulatory CpG and 25 μg/mL I18 (CpG+I18(25)), and cytokine levels were analyzed by ELISA. I18 suppressed both CpG stimulated IL-6 (A) and IL-10 (B) expression. C. CD19+ B cells were incubated alone (Cells only) with 5 μg/mL stimulatory CpG (CpG-5), with 5 μg/mL stimulatory CpG in the presence of 1 μg/mL (CpG+I18-1), 5 μg/mL (CpG+I18-5) or 25 μg/mL (CpG+I18-25) I18. Cell proliferation was assayed by [³H] thymidine incorporation. I18 significantly suppressed CpG stimulated C cell proliferation at all doses.

FIG. 37: I18 Activates Expression of IL-6 in Normal B Cells. A. Isolated

CD19+CD27+ memory B cells were incubated alone (no dna), with 5 μg/mL CpG (CpG(5)), with 5 μg/mL I18 (I18(5)) or with 25 μg/mL I18 (I18(25)) and IL-6 expression analyzed by ELISA. I18 induces lower level expression of IL-6 in memory B cells compared to CpG sequences. B. Isolated CD19+CD27− naive B cells were incubated alone (no dna), with 5 μg/mL CpG (CpG(5)), with 5 μg/mL I18 (I18(5)) or with 25 μg/mL I18 (I18(25) and IL-6 expression analyzed by ELISA. I18 activates IL-6 expression in naïve B cells to a similar degree as CpG sequences.

FIG. 38: I18 Activates Expression of IL-10 in Normal B Cells. A. Isolated CD19+CD27+ memory B cells were incubated alone (no dna), with 5 μg/mL CpG (CpG(5)), with 5 μg/mL I18 (I18(5)) or with 25 μg/mL I18 (I18(25)) and IL-10 expression analyzed by ELISA. I18 induces lower level expression of IL-10 in memory B cells compared to CpG sequences. B. Isolated CD19+CD27− naive B cells were incubated alone (no dna), with 5 μg/mL CpG (CpG(5)), with 5 μg/mL I18 (I18(5)) or with 25 μg/mL I18 (I18(25)) and IL-10 expression analyzed by ELISA. I18 induces lower level expression of IL-10 in naïve B cells compared to CpG sequences.

FIG. 39: I18 Activates Expression of Co-Stimulatory Markers in Normal B Cells. Isolated CD19+ B cells were incubated alone (no dna), with 5 μg/mL CpG alone (CpG-1826) or in the presence of Chloroquine (CpG-1826+Ch1), or with 5 μg/mL I18 alone (I18) or in the presence of Chloroquine (I1+Ch1). Expression of CD80 and CD86 was determined by FACs and the percentage of cells expressing each co-stimulatory marker is shown. I18 activates expression CD80 and CD86 at lower levels than CpG sequences.

FIG. 40: I18 Does Not Stimulate Long Term Survival or Proliferation of Normal B Cells. Isolated CD19+ B cells were incubated alone (Cell Only); with 1 μg/mL of three different CpG sequence (1018; 2395; 2006); or 0.2 μg/mL, 1 μg/mL or 5 μg/mL I18. The starting concentration of cells is indicated and the total number of cells under each condition after 13 days graphed. I18 did not increase survival or proliferation of B cells.

FIG. 41: I18 is a Weak Activator of Lupus B Cells. Isolated CD19+ B cells from a lupus patient were incubated alone (No dna); with 1 μg/mL, 5 μg/mL, or 25 μg/mL I18, with 5 μg/mL CpG; or with 5 μg/mL CpG on the presence of 5 μg/mL or 25 μg/mL I18. IL-6 expression (A), IL-10 expression (B) and cell proliferation (C) were analyzed. I18 weakly activated expression of both IL-6 and IL-10, and slightly increased cell proliferation.

FIG. 42: I18 Administration in a SLE Animal Model Decreases the Percentage of Animals that Develop anti-dsDNA Antibodies. I18 IMS oligos were administered to NZB/W F1 female mice weekly at 10 μg, 50 μg and 250 μg by intradermal delivery. The percentage of animals with anti-dsDNA antibodies was graphed compared to PBS negative controls and steroid positive controls (Depo+Cytoxan). The percentage of animals with anti-dsDNA antibodies was statistically less in the groups receiving 50 μg (p=0.17) and 250 μg (p=0.04) weekly doses of I18.

FIG. 43: I18 Administration in a SLE Animal Model Delays Disease Onset. NZB/W F1 females were administered 10 μg, 50 μg, or 250 μg I18 daily, 3× weekly or weekly for a total of 45 weeks. Proteinuria onset was assessed and the percentage of animals with proteinuria shown for each group over time. A. Administration of 10 μg I18 did not affect disease onset. B. Daily, 3× weekly and weekly administration of 50 μg I18 showed a trend towards decreased disease onset compared to PBS controls. C. Weekly and 3× weekly administration of 250 μg I18 showed a trend towards decreased disease onset compared to PBS controls.

FIG. 44: I18 Administration at 250 μg in a SLE Animal Delays Disease Onset. NZB/W F1 females were administered 10 μg, 50 μg, or 250 μg I18 daily, 3× weekly or weekly for a total of 45 weeks. Proteinuria onset was assessed and the percentage of animals with proteinuria shown for each group over time. A. Daily administration of I18 at 10 μg or 50 μg did not affect disease onset. B. Administration of I18 3× weekly at 250 μg showed a statistically significant trend (LogRank Test p=0.31) compared to administration with 10 μg and 50 μg I18. C. Weekly administration of I18 at 250 μg showed a statistically significant trend (LogRank Test p=0.03) compared to administration with 10 μg and 50 μg I18.

FIG. 45: I18-Derived Oligonucleotides Inhibit CpG Stimulated Production of IL-6 by Human B Cells. Isolated human B-cells were incubated for 48 hours with 5 μg/mL stimulatory CpG-ODN or I18-derived oligonucleotides alone (left columns) or with stimulatory CpG-ODN in the presence of 5 μg/mL I18 or I18-derived oligonucleotides (right columns). Cytokine levels in the culture medium were analyzed by ELISA and recorded as μg/ml on the y-axis.

FIG. 46 illustrates a sequence comparison between I18 and M49.

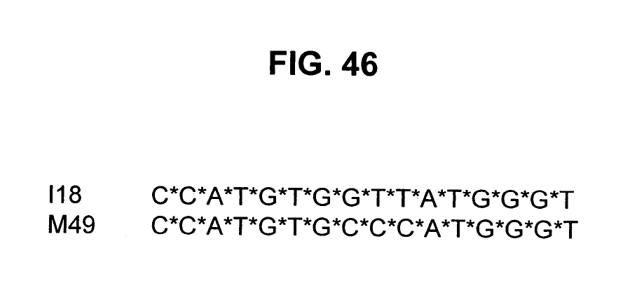

FIG. 47 illustrates a comparison of I18 and M49 inhibitory activity in vitro: Mouse splenocytes were isolated from healthy C57B1/6 mice and cultured at a density of 1×10⁶ cells/ml in the presence of a) TLR9 (CpG oligo 1018 at 10 μg/ml) and b) TLR7 (gardiquimod at 1 μg/ml) agonists and a dose range of inhibitory oligonucleotides. The inhibitory oligos and the agonists were added simultaneously to the culture. Culture supernatants were isolated 24 hours after the addition of oligo and agonists and IL-6 levels were determined using a commercial ELISA kit. The % inhibition was determined by calculating the amount of IL-6 levels for each oligo dose relative to the level of agonist alone. The I18 compound is a modestly better inhibitor of both TLR9 and TLR7 in these assays.

FIG. 48 illustrates a comparison of I18 and M49 inhibitory activity in vivo: Compared to I18, M49 has modestly decreased TLR9 inhibitory activity and decreased B cell agonist activity assessed in CD40L synergy assay. It has efficacy in the NZB/W model improving survival rate and lowering proteinurea scores and anti-dsDNA antibody titers superior to I18. M49 shows that sequence changes in hexamer core region affect activity as substitution of “CCC” vs “GTT” in I18. Increased efficacy of M49 in NZB model and decreased agonist activity. M49 is less effective TLR9 inhibitor in splenocyte assays but has better in vivo efficacy. NZB/W F1 mice (n=15 per group) were given a subcutaneous injection of 250 μg (0.05 mL of a 5 mg/ml PBS solution) of the oligonucleotide (I18, M49) once per week beginning at 21 weeks of age and continuing to 40 weeks of age. Control mice were dosed weekly with 0.05 mLs of PBS. A pre-bleed and monthly bleeds were taken for autoantibody profiling and proteinurea levels were measured weekly. Weights were measured and animals were euthanized after a 25% decrease in body weight was observed. The M49 oligo treatment resulted in a decrease in proteinurea levels (a) complete prevention of lethality (b), and a reduction in anti-dsDNA antibody levels (c) as measured by a commercial ELISA kit at the termination point of the study (20 weeks of treatment).

FIG. 49 illustrates decreased activation of human B cells incubated with a combination of recombinant CD40 ligand and oligonucleotide M49. Human B cells purified from the blood of healthy donors were incubated with recombinant CD40 ligand alone or in the presence of a 1 μM dose of inhibitory oligonucleotide (I18 or M49).

Supernatants were removed from the cultures after a 24-hour incubation and the levels of IL-6 protein were measured by ELISA.

### DETAILED DESCRIPTION OF THE INVENTION

Before describing the present invention in detail, it is to be understood that this invention is not limited to particular formulations or process parameters as they may, of course, vary. It is also to be understood that the terminology used herein is for the purpose of describing particular embodiments of the invention only, and is not intended to be limiting.

### Definitions

“Nucleic acid” and “polynucleotide” as used herein are synonymous and refer to a polymer of nucleotides (e.g., deoxynucleotide, ribonucleotide, or analog thereof, including single or double stranded forms).

“Oligonucleotide” as used herein refers to a subset of nucleic acid of from about 6 to about 175 nucleotides or more in length. Typical oligonucleotides of the invention are from about 14 up to about 50, 75 or 100 nucleotides in length. Oligonucleotide refers to both oligoribonucleotides and to oligodeoxyribonucleotides, herein after referred to ODNs. ODNs include oligonucleosides and other organic base containing polymers.

Nucleotides are molecules comprising a sugar (preferably ribose or deoxyribose) linked to a phosphate group and an exchangeable organic base, which can be either a substituted purine (guanine (G), adenine (A), or inosine (I)) or a substituted pyrimidine (thymine (T), cytosine (C), or uracil (U)).

Immune Modulatory Sequences (IMSs). “Immune modulatory sequence” or “IMS” as used herein refers to a sequence of nucleotides of a nucleic acid or region of a nucleic acid that is capable of modulating an autoimmune or inflammatory disease. An IMS may be, for example, an oligonucleotide or a sequence of nucleotides incorporated in a vector, for example an expression vector. An IMS of the invention is typically from about 14 to about 50 nucleotides in length, more usually from about 15 to about 30 nucleotides. An “immune modulatory nucleic acid” as used herein means a nucleic acid molecule that comprises one or more IMSs. The term IMS is used interchangeably with immune inhibitory sequence (IIS).

The terms “identity” or “percent identity” in the context of two or more nucleic acid or polypeptide sequences, refer to two or more sequences or subsequences that are the same or have a specified percentage of amino acid residues or nucleotides that are the same, when compared and aligned for maximum correspondence, as measured using either a sequence comparison algorithm such as, e.g., PILEUP or BLAST or a similar algorithm (See, e.g., Higgins and Sharp, CABIOS, 5:151-153, 1989; Altschul et al., J. Mol. Biol., 215:403-410,1990). Optimal alignment of sequences for comparison can be conducted, e.g., by the local homology algorithm of Smith & Waterman, Adv. Appl. Math., 2:482, 1981, by the homology alignment algorithm of Needleman & Wunsch, J. Mol. Biol., 48:443, 1970, by the search for similarity method of Pearson & Lipman, Proc. Nat'l. Acad. Sci. USA, 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see, generally, Ausubel et al., supra).

The phrase “substantially identical,” in the context of two nucleic acids or polypeptides, refers to two or more sequences or subsequences that have at least 60%, preferably at least about 70%, more preferably at least about 80%, and most preferably at least about 90% or at least about 95%, 97% or 99% nucleotide or amino acid residue identity, when compared and aligned for maximum correspondence. Preferably, the substantial identity exists over a region of the sequences that is at least about 50 residues in length, more preferably over a region of at least about 100 residues, and most preferably the sequences are substantially identical over at least about 150 residues. In a preferred embodiment, the sequences are substantially identical over the entire length of a given nucleic acid or polypeptide. In certain embodiments of the invention, a nucleic acid or polypeptide (e.g., self-protein, -polypeptide, or -peptide or a nucleic acid encoding the self-protein, -polypeptide, or -peptide) is substantially identical to a specific nucleic acid or polypeptide disclosed herein.

“Self-molecules” as used herein include self-lipids, self-antigen(s), self-proteins(s), self-peptide(s), self-polypeptide(s), self-glycolipid(s), self-carbohydrate(s), self-glycoprotein(s), and posttranslationally-modified self- protein(s), peptide(s), polypeptide(s), or glycoprotein(s). “Self protein(s), polypeptide(s), or peptide(s), or fragment(s) or derivative(s)” includes protein(s), polypeptide(s) or peptide(s) encoded within the genome of the animal; is produced or generated in the animal; may be modified posttranslationally at some time during the life of the animal; or is present in the animal non-physiologically. The term “non-physiological” or “non-physiologically” when used to describe the self-proteins, -polypeptides, or -peptides of this invention means a departure or deviation from the normal role or process in the animal for that self-protein, -polypeptide or -peptide. Self-antigen(s), self-proteins(s), -polypeptide(s) or -peptides of this invention also referred to as autoantigens. When referring to the self-protein, -polypeptide or -peptide as “associated with a disease” or “involved in a disease” it is understood to mean that the self-protein, -polypeptide, or -peptide may be modified in form or structure and thus be unable to perform its physiological role or process; or may be involved in the pathophysiology of the condition or disease either by inducing the pathophysiology, mediating or facilitating a pathophysiologic process; and/or by being the target of a pathophysiologic process. For example, in autoimmune disease, the immune system aberrantly attacks self-molecules such as self-lipids, self-antigen(s), self-proteins(s), self-peptide(s), self-polypeptide(s), self-glycolipid(s), self-carbohydrate(s), self-glycoprotein(s), and posttranslationally-modified self- protein(s), peptide(s), polypeptide(s), or glycoprotein(s), causing damage and dysfunction of cells and tissues in which the self-molecule is expressed and/or present. Alternatively, the molecule can itself be expressed at non-physiological levels and/or function non-physiologically. For example in neurodegenerative diseases self-proteins are aberrantly expressed, and aggregate in lesions in the brain thereby causing neural dysfunction. In other cases, the self-molecule aggravates an undesired condition or process. For example in osteoarthritis, self-proteins including collagenases and matrix metalloproteinases aberrantly degrade cartilage covering the articular surface of joints. Examples of posttranslational modifications of self-antigen(s), -proteins(s), -polypeptide(s) or -peptide(s) are glycosylation, addition of lipid groups, dephosphorylation by phosphatases, addition of dimethylarginine residues, citrullination of fillagrin and fibrin by peptidyl arginine deiminase (PAD); alpha B-crystallin phosphorylation; citrullination of MBP; and SLE autoantigen proteolysis by caspases and granzymes. Immunologically, self-protein, -polypeptide or -peptide would all be considered host self-antigen(s) and under normal physiological conditions are ignored by the host immune system through the elimination, inactivation, or lack of activation of immune cells that have the capacity to recognize self-antigen(s) through a process designated “immune tolerance.” Self-protein, -polypeptide, or -peptide does not include immune proteins, polypeptides, or peptides which are molecules expressed physiologically, specifically and exclusively by cells of the immune system for the purpose of regulating immune function. The immune system is the defense mechanism that provides the means to make rapid, highly specific, and protective responses against the myriad of potentially pathogenic microorganisms inhabiting the animal's world. Examples of immune protein(s), polypeptide(s) or peptide(s) are proteins comprising the T-cell receptor, immunoglobulins, cytokines including the type I interleukins, and the type II cytokines, including the interferons and IL-10, TNF-α, lymphotoxin, and the chemokines such as macrophage inflammatory protein -1alpha and beta, monocyte-chemotactic protein and RANTES, and other molecules directly involved in immune function such as Fas-ligand. There are certain immune proteins, polypeptide(s) or peptide(s) that are included in the self-protein, -polypeptide or _peptide of the invention and they are: class I MHC membrane glycoproteins, class II MHC glycoproteins and osteopontin. Self-protein, -polypeptide or -peptide does not include proteins, polypeptides, and peptides that are absent from the subject, either entirely or substantially, due to a genetic or acquired deficiency causing a metabolic or functional disorder, and are replaced either by administration of said protein, polypeptide, or peptide or by administration of a polynucleotide encoding said protein, polypeptide or peptide (gene therapy). Examples of such disorders include Duchenne' muscular dystrophy, Becker's muscular dystrophy, cystic fibrosis, phenylketonuria, galactosemia, maple syrup urine disease, and homocystinuria. Self-protein, -polypeptide or -peptide does not include proteins, polypeptides, and peptides expressed specifically and exclusively by cells which have characteristics that distinguish them from their normal counterparts, including: (1) clonality, representing proliferation of a single cell with a genetic alteration to form a clone of malignant cells, (2) autonomy, indicating that growth is not properly regulated, and (3) anaplasia, or the lack of normal coordinated cell differentiation. Cells have one or more of the foregoing three criteria are referred to either as neoplastic, cancer or malignant cells.

“Plasmids” and “vectors” are designated by a lower case p followed by letters and/or numbers. The starting plasmids are commercially available, publicly available on an unrestricted basis, or can be constructed from available plasmids in accord with published procedures. In addition, equivalent plasmids to those described are known in the art and will be apparent to the ordinarily skilled artisan. A “vector” or “plasmid” refers to any genetic element that is capable of replication by comprising proper control and regulatory elements when present in a host cell. For purposes of this invention examples of vectors or plasmids include, but are not limited to, plasmids, phage, transposons, cosmids, virus, and the like.

“Naked nucleic acid” as used herein refers to a nucleic acid molecule that is not encapsulated (such as, e.g., within a viral particle, bacterial cell, or liposome) and not complexed with a molecule that binds to the nucleic acid (such as, e.g., DEAE-dextran) nor otherwise conjugated to the nucleic acid (e.g., gold particles or polysaccharide-based supports).

“Treating,” “treatment,” or “therapy” of a disease or disorder shall mean slowing, stopping or reversing the progression of established disease, as evidenced by a decrease, cessation or elimination of either clinical or diagnostic symptoms, by administration of the immune modulatory nucleic acid of this invention. “Established disease” means the immune system is active, causing the affected tissues to be inflamed and abnormally infiltrated by leukocytes and lymphocytes. “Treating,” “treatment,” or “therapy” of a disease or disorder shall also mean slowing, stopping or reversing the disease's progression by administration of an immune modulatory nucleic acid in combination with a self-molecule. “Self-molecules” as used herein refer to self-lipids, self-antigen(s), self-proteins(s), self-peptide(s), self-polypeptide(s), self-glycolipid(s), self-carbohydrate(s), self-glycoprotein(s), and posttranslationally-modified self- protein(s), peptide(s), polypeptide(s), or glycoprotein(s). “Treating,” “treatment,” or “therapy” of a disease or disorder shall further mean slowing, stopping or reversing the disease's progression by administration of an immune modulatory nucleic acid in combination with an immune modulatory therapeutic. “In combination with” when referring to a therapeutic regimen comprising an immune modulatory nucleic acid and another compound, for example DNA encoding a self-protein, -peptide, or -polypeptide, includes two or more compounds administered separately but together physically as co-administration in a vial, linked together as for example by conjugation, encoded by DNA on one or more vectors, or administered separately at different sites but temporally so close together to be considered by one of ordinary skill in the art to be administered “in combination.” As used herein, ameliorating a disease and treating a disease are equivalent.

“Preventing,” “prophylaxis” or “prevention” of a disease or disorder as used in the context of this invention refers to the administration of a immune modulatory sequence either alone or in combination with another compound as described herein, to prevent the occurrence or onset of a disease or disorder or some or all of the symptoms of a disease or disorder or to lessen the likelihood of the onset of a disease or disorder. “Preventing,” “prophylaxis” or “prevention” of a disease or disorder as used in the context of this invention refers to the administration of an immune modulatory sequence in combination with self-molecules to prevent the occurrence or onset of a disease or disorder or some or all of the symptoms of a disease or disorder or to lessen the likelihood of the onset of a disease or disorder. “Preventing,” “prophylaxis” or “prevention” of a disease or disorder as used in the context of this invention refers to the administration of an immune modulatory sequence in combination with an immune modulatory therapeutic to prevent the occurrence or onset of a disease or disorder or some or all of the symptoms of a disease or disorder or to lessen the likelihood of the onset of a disease or disorder. As used herein “immune modulatory therapeutics” refers to such molecules that have an immune modulatory or regulatory function when administered to a subject. Such immune modulatory therapeutics include cytokines, chemokines, steroids, or antibodies to antigens or autoantigens.

“Subjects” shall mean any animal, such as, for example, a human, non-human primate, horse, cow, dog, cat, mouse, rat, guinea pig or rabbit.

### Autoimmune Diseases

The compositions and methods described herein are useful for the treatment or prevention of autoimmune disease. Several examples of autoimmune diseases associated with self molecules including self-lipids, self-antigen(s), self-proteins(s), self-peptide(s), self-polypeptide(s), self-glycolipid(s), self-carbohydrate(s), self-glycoprotein(s), and posttranslationally-modified self- protein(s), peptide(s), polypeptide(s), glycoprotein(s), or derivatives of self molecules present in the animal non-physiologically is set forth in the table below and is described below.

TABLE 1

| Autoimmune | Tissue | Self-Protein(s) Associated With An Autoimmune |
|---|---|---|
| Disease | Targeted | Disease |
| Multiple | central | myelin basic protein, proteolipid protein, myelin |
| sclerosis | nervous | associated glycoprotein, cyclic nucleotide |
| | system | phosphodiesterase, yelin-associated glycoprotein, |
| | | myelin-associated oligodendrocytic basic protein; alpha- |
| | | B-crystalin; myelin oligodendrocyte glycoprotein |
| Guillian Barre | peripheral | peripheral myelin protein I and others |
| Syndrome | nerv. sys. | |
| Insulin | Beta cells in | tyrosine phosphatase IA2, IA-2β; glutamic acid |
| Dependent | islets of | decarboxylase (65 and 67 kDa forms), carboxypeptidase |
| Diabetes | pancreas | H, insulin, proinsulin, heat shock proteins, glima 38, |
| Mellitus | | islet cell antigen 69 KDa, p52, islet cell glucose |
| | | transporter GLUT-2 |
| Rheumatoid | synovial joints | Immunoglobulin, fibrin, filaggrin, type I, II, III, IV, V, |
| Arthritis | | IX, and XI collagens, GP-39, hnRNPs |
| Autoimmune | iris, uveal tract | S-antigen, interphotoreceptor retinoid binding protein |
| Uveitis | | (IRBP), rhodopsin, recoverin |
| Primary | biliary tree of | pyruvate dehydrogenase complexes (2-oxoacid |
| Biliary | liver | dehydrogenase) |
| Cirrhosis | | |
| Autoimmune | Liver | Hepatocyte antigens, cytochrome P450 |
| Hepatitis | | |
| Pemphigus | Skin | Desmoglein-1, -3, and others |
| vulgaris | | |
| Myasthenia | nerve-muscle | acetylcholine receptor |
| Gravis | junct. | |
| Autoimmune | stomach/parietal | H⁺/K⁺ ATPase, intrinsic factor |
| gastritis | cells | |
| Pernicious | Stomach | intrinsic factor |
| Anemia | | |
| Polymyositis | Muscle | histidyl tRNA synthetase, other synthetases, other |
| | | nuclear antigens |
| Autoimmune | Thyroid | Thyroglobulin, thyroid peroxidase |
| Thyroiditis | | |
| Graves's | Thyroid | Thyroid-stimulating hormone receptor |
| Disease | | |
| Psoriasis | Skin | Unknown |
| Vitiligo | Skin | Tyrosinase, tyrosinase-related protein-2 |
| Systemic | Systemic | nuclear antigens: DNA, histones, ribonucleoproteins |
| Lupus Eryth. | | |
| Celiac Disease | Small bowel | Transglutaminase |

Multiple Sclerosis: Multiple sclerosis (MS) is the most common demyelinating disorder of the central nervous system (CNS) and affects 350,000 Americans and one million people worldwide. See, e.g., Cohen and Rudick (eds. 2007) Multiple Sclerosis Therapeutics (3d ed) Informa Healthcare, ISBN-10: 1841845256, ISBN-13: 978-1841845258; Matthews and Margaret Rice-Oxley (2006) Multiple Sclerosis: The Facts (Oxford Medical Publications 4th Ed.) Oxford University Press, USA, ISBN-10: 0198508980, ISBN-13: 978-0198508984; Cook (ed. 2006) Handbook of Multiple Sclerosis (Neurological Disease and Therapy, 4th Ed.) Informa Healthcare, ISBN-10: 1574448277, ISBN-13: 978-1574448276; Compston, et al. (2005) McAlpine's Multiple Sclerosis (4th edition) Churchill Livingstone, ISBN-10: 044307271X, ISBN-13: 978-0443072710; Burks and Johnson (eds 2000) Multiple Sclerosis: Diagnosis, Medical Management, and Rehabilitation Demos Medical Publishing ISBN-10: 1888799358, ISBN-13: 978-1888799354; Waxman (2005) Multiple Sclerosis As A Neuronal Disease Academic Press ISBN-10: 0127387617, ISBN-13: 978-0127387611; Filippi, et al. (eds.) Magnetic Resonance Spectroscopy in Multiple Sclerosis (Topics in Neuroscience) Springer, ISBN-10: 8847001234, ISBN-13: 978-8847001237; Herndon (ed. 2003) Multiple Sclerosis: Immunology, Pathology and Pathophysiology Demos Medical Publishing, ISBN-10: 1888799625, ISBN-13: 978-1888799620; Costello, et al. (2007) “Combination therapies for multiple sclerosis: scientific rationale, clinical trials, and clinical practice” Curr. Opin. Neurol. 20(3):281-285, PMID: 17495621; Burton and O'connor (2007) “Novel Oral Agents for Multiple Sclerosis” Curr. Neurol. Neurosci. Rep. 7(3):223-230, PMID: 17488588; Correale and Villa (2007) “The blood-brain-barrier in multiple sclerosis: functional roles and therapeutic targeting” Autoimmunity 40(2):148-60, PMID: 17453713; De Stefano, et al. (2007) “Measuring brain atrophy in multiple sclerosis” J. Neuroimaging 17 Suppl 1:10S-15S, PMID: 17425728; Neema, et al. (2007) “T1- and T2-based MRI measures of diffuse gray matter and white matter damage in patients with multiple sclerosis” J. Neuroimaging 17 Suppl 1:16S-21 S, PMID: 17425729; De Stefano and Filippi (2007) “MR spectroscopy in multiple sclerosis” J. Neuroimaging 17 Suppl 1:31S-35S, PMID: 17425732; and Comabella and Martin (2007) “Genomics in multiple sclerosis-Current state and future directions” J. Neuroimmunol. Epub ahead of print] PMID: 17400297.

Onset of symptoms typically occurs between 20 and 40 years of age and manifests as an acute or sub-acute attack of unilateral visual impairment, muscle weakness, paresthesias, ataxia, vertigo, urinary incontinence, dysarthria, or mental disturbance (in order of decreasing frequency). Such symptoms result from focal lesions of demyelination which cause both negative conduction abnormalities due to slowed axonal conduction, and positive conduction abnormalities due to ectopic impulse generation (e.g. Lhermitte's symptom). Diagnosis of MS is based upon a history including at least two distinct attacks of neurologic dysfunction that are separated in time, produce objective clinical evidence of neurologic dysfunction, and involve separate areas of the CNS white matter. Laboratory studies providing additional objective evidence supporting the diagnosis of MS include magnetic resonance imaging (MRI) of CNS white matter lesions, cerebral spinal fluid (CSF) oligoclonal banding of IgG, and abnormal evoked responses. Although most patients experience a gradually progressive relapsing remitting disease course, the clinical course of MS varies greatly between individuals and can range from being limited to several mild attacks over a lifetime to fulminant chronic progressive disease. A quantitative increase in myelin-autoreactive T cells with the capacity to secrete IFN-gamma is associated with the pathogenesis of MS and EAE.

Rheumatoid Arthritis: Rheumatoid arthritis (RA) is a chronic autoimmune inflammatory synovitis affecting 0.8% of the world population. It is characterized by chronic inflammatory synovitis that causes erosive joint destruction. See, e.g., St. Clair, et al. (2004) Rheumatoid Arthritis Lippincott Williams & Wilkins, ISBN-10: 0781741491, ISBN-13: 978-0781741491; Firestein, et al. (eds. 2006) Rheumatoid Arthritis (2d Ed.) Oxford University Press, USA, ISBN-10: 0198566301, ISBN-13: 978-0198566304; Emery, et al. (2007) “Evidence-based review of biologic markers as indicators of disease progression and remission in rheumatoid arthritis” Rheumatol. Int. [Epub ahead of print] PMID: 17505829; Nigrovic, et al. (2007) “Synovial mast cells: role in acute and chronic arthritis” Immunol. Rev. 217(1):19-37, PMID: 17498049; and Manuel, et al. (2007) “Dendritic cells in autoimmune diseases and neuroinflammatory disorders” Front. Biosci. 12:4315-335, PMID: 17485377. RA is mediated by T cells, B cells and macrophages.

Evidence that T cells play a critical role in RA includes the (1) predominance of CD4+ T cells infiltrating the synovium, (2) clinical improvement associated with suppression of T cell function with drugs such as cyclosporine, and (3) the association of RA with certain HLA-DR alleles. The HLA-DR alleles associated with RA contain a similar sequence of amino acids at positions 67-74 in the third hypervariable region of the beta chain that are involved in peptide binding and presentation to T cells. RA is mediated by autoreactive T cells that recognize a self molecule such as self-lipids, self-antigen(s), self-proteins(s), self-peptide(s), self-polypeptide(s), self-glycolipid(s), self-carbohydrate(s), self-glycoprotein(s), and posttranslationally-modified self- protein(s), peptide(s), polypeptide(s), or glycoprotein(s), or an unidentified self biomolecule present in synovial joints or elsewhere in the host. Self-antigen(s), self-proteins(s), -polypeptide(s) or -peptides of this invention also referred to as autoantigens are targeted in RA and comprise epitopes from type II collagen; hnRNP; A2/RA33; Sa; filaggrin; keratin; citrulline; cartilage proteins including gp39; collagens type I, III, IV, V, IX, XI; HSP-65/60; IgM (rheumatoid factor); RNA polymerase; hnRNP-B 1; hnRNP-D; cardiolipin; aldolase A; citrulline-modified filaggrin and fibrin. Autoantibodies that recognize filaggrin peptides containing a modified arginine residue (de-iminated to form citrulline) have been identified in the serum of a high proportion of RA patients. Autoreactive T and B cell responses are both directed against the same immunodominant type II collagen (CII) peptide 257-270 in some patients.

Insulin Dependent Diabetes Mellitus: Human type I or insulin-dependent diabetes mellitus (IDDM) is characterized by autoimmune destruction of the Beta cells in the pancreatic islets of Langerhans. The depletion of Beta cells results in an inability to regulate levels of glucose in the blood. See, e.g., Sperling (ed. 2001) Type 1 Diabetes in Clinical Practice (Contemporary Endocrinology) Humana Press, ISBN-10: 0896039315, ISBN-13: 978-0896039315; Eisenbarth (ed. 2000); Type 1 Diabetes: Molecular, Cellular and Clinical Immunology (Advances in Experimental Medicine and Biology) Springer, ISBN-10: 0306478714, ISBN-13: 978-0306478710; Wong and Wen (2005) “B cells in autoimmune diabetes” Rev. Diabet. Stud. 2(3):121-135, Epub 2005 Nov. 10, PMID: 17491687; Sia (2004) “Autoimmune diabetes: ongoing development of immunological intervention strategies targeted directly against autoreactive T cells” Rev. Diabet. Stud. 1(1):9-17, Epub 2004 May 10, PMID: 17491660; Triplitt (2007) “New technologies and therapies in the management of diabetes” Am. J. Manag. Care 13(2 Suppl):S47-54, PMID: 17417933; and Skyler (2007) “Prediction and prevention of type 1 diabetes: progress, problems, and prospects” Clin. Pharmacol. Ther. 81(5):768-71, Epub 2007 Mar. 28, PMID: 17392722.

Overt diabetes occurs when the level of glucose in the blood rises above a specific level, usually about 250 mg/dl. In humans a long presymptomatic period precedes the onset of diabetes. During this period there is a gradual loss of pancreatic beta cell function. The development of disease is implicated by the presence of autoantibodies against insulin, glutamic acid decarboxylase, and the tyrosine phosphatase IA2 (IA2), each an example of a self-protein, -polypeptide or -peptide according to this invention.

Markers that may be evaluated during the presymptomatic stage are the presence of insulitis in the pancreas, the level and frequency of islet cell antibodies, islet cell surface antibodies, aberrant expression of Class II MHC molecules on pancreatic beta cells, glucose concentration in the blood, and the plasma concentration of insulin. An increase in the number of T lymphocytes in the pancreas, islet cell antibodies and blood glucose is indicative of the disease, as is a decrease in insulin concentration.

The Non-Obese Diabetic (NOD) mouse is an animal model with many clinical, immunological, and histopathological features in common with human IDDM. NOD mice spontaneously develop inflammation of the islets and destruction of the Beta cells, which leads to hyperglycemia and overt diabetes. Both CD4+ and CD8+ T cells are required for diabetes to develop, although the roles of each remain unclear. It has been shown with both insulin and GAD that when administered as proteins under tolerizing conditions, disease can be prevented and responses to the other self-antigen(s) downregulated.

Importantly, NOD mice develop autoimmune diabetes in clean pathogen-free mouse houses, and in germ-free environments.

Human IDDM is currently treated by monitoring blood glucose levels to guide injection, or pump-based delivery, of recombinant insulin. Diet and exercise regimens contribute to achieving adequate blood glucose control.

Autoimmune Uveitis: Autoimmune uveitis is an autoimmune disease of the eye that is estimated to affect 400,000 people, with an incidence of 43,000 new cases per year in the U.S. Autoimmune uveitis is currently treated with steroids, immunosuppressive agents such as methotrexate and cyclosporin, intravenous immunoglobulin, and TNFalpha-antagonists. See, e.g., Pleyer and Mondino (eds. 2004) Uveitis and Immunological Disorders (Essentials in Ophthalmology) Springer, ISBN-10: 3540200452, ISBN-13: 978-3540200451; Vallochi, et al. (2007) “The role of cytokines in the regulation of ocular autoimmune inflammation” Cytokine Growth Factor Rev. 18(1-2):135-141, Epub 2007 Mar. 8, PMID: 17349814; Bora and Kaplan (2007) “Intraocular diseases—anterior uveitis” Chem. Immunol. Allergy. 92:213-20, PMID: 17264497; and Levinson (2007) “Immunogenetics of ocular inflammatory disease” Tissue Antigens 69(2):105-112, PMID: 17257311.

Experimental autoimmune uveitis (EAU) is a T cell-mediated autoimmune disease that targets neural retina, uvea, and related tissues in the eye. EAU shares many clinical and immunological features with human autoimmune uveitis, and is induced by peripheral administration of uveitogenic peptide emulsified in Complete Freund's Adjuvant (CFA).

Self-proteins targeted by the autoimmune response in human autoimmune uveitis may include S-antigen, interphotoreceptor retinoid binding protein (IRBP), rhodopsin, and recoverin.

Primary Biliary Cirrhosis Primary Biliary Cirrhosis (PBC) is an organ-specific autoimmune disease that predominantly affects women between 40-60 years of age. The prevalence reported among this group approaches 1 per 1,000. PBC is characterized by progressive destruction of intrahepatic biliary epithelial cells (IBEC) lining the small intrahepatic bile ducts. This leads to obstruction and interference with bile secretion, causing eventual cirrhosis. Association with other autoimmune diseases characterized by epithelium lining/secretory system damage has been reported, including Sjogren's Syndrome, CREST Syndrome, Autoimmune Thyroid Disease and Rheumatoid Arthritis. Attention regarding the driving antigen(s) has focused on the mitochondria for over 50 years, leading to the discovery of the antimitochondrial antibody (AMA) (Gershwin et al., Immunol Rev, 174:210-225 (2000); Mackay et al., Immunol Rev, 174:226-237 (2000)). AMA soon became a cornerstone for laboratory diagnosis of PBC, present in serum of 90-95% patients long before clinical symptoms appear. Autoantigenic reactivities in the mitochondria were designated as M1 and M2. M2 reactivity is directed against a family of components of 48-74 kDa. M2 represents multiple autoantigenic subunits of enzymes of the 2-oxoacid dehydrogenase complex (2-OADC) and is another example of the self-protein, -polypeptide, or -peptide of the instant invention.

Studies identifying the role of pyruvate dehydrogenase complex (PDC) antigens in the etiopathogenesis of PBC support the concept that PDC plays a central role in the induction of the disease (Gershwin et al., Immunol Rev, 174:210-225 (2000); Mackay et al., Immunol Rev, 174:226-237 (2000)). The most frequent reactivity in 95% of cases of PBC is the E2 74 kDa subunit, belonging to the PDC-E2. There exist related but distinct complexes including: 2-oxoglutarate dehydrogenase complex (OGDC) and branched-chain (BC) 2-OADC. Three constituent enzymes (E1, 2, 3) contribute to the catalytic function which is to transform the 2-oxoacid substrate to acyl co-enzyme A (CoA), with reduction of NAD to NADH. Mammalian PDC contains an additional component, termed protein X or E-3 Binding protein (E3BP). In PBC patients, the major antigenic response is directed against PDC-E2 and E3BP. The E2 polypeptide contains two tandemly repeated lipoyl domains, while E3BP has a single lipoyl domain. PBC is treated with glucocorticoids and immunosuppressive agents including methotrexate and cyclosporin A. See, e.g., Sherlock and Dooley (2002) Diseases of the Liver & Biliary System (11th ed.) Blackwell Pub., ISBN-10: 0632055820, ISBN-13: 978-0632055821; Boyer, et al. (eds. 2001) Liver Cirrhosis and its Development (Falk Symposium, Volume 115) Springer, ISBN-10: 0792387600, ISBN-13: 978-0792387602; Crispe (ed. 2001) T Lymphocytes in the Liver: Immunobiology, Pathology and Host Defense Wiley-Liss, ISBN-10: 047119218X, ISBN-13: 978-0471192183; Lack (2001) Pathology of the Pancreas, Gallbladder, Extrahepatic Biliary Tract, and Ampullary Region (Medicine) Oxford University Press, USA, ISBN-10: 0195133927, ISBN-13: 978-0195133929; Gong, et al. (2007) “Ursodeoxycholic Acid for Patients With Primary Biliary Cirrhosis: An Updated Systematic Review and Meta-Analysis of Randomized Clinical Trials Using Bayesian Approach as Sensitivity Analyses” Am. J. Gastroenterol. [Epub ahead of print] PMID: 17459023; Lazaridis and Talwalkar (2007) “Clinical Epidemiology of Primary Biliary Cirrhosis: Incidence, Prevalence, and Impact of Therapy” J. Clin. Gastroenterol. 41(5):494-500, PMID: 17450033; and Sorokin, et al. (2007) “Primary biliary cirrhosis, hyperlipidemia, and atherosclerotic risk: A systematic review” Atherosclerosis [Epub ahead of print] PMID: 17240380.

A murine model of experimental autoimmune cholangitis (EAC) uses intraperitoneal (i.p.) sensitization with mammalian PDC in female SJL/J mice, inducing non-suppurative destructive cholangitis (NSDC) and production of AMA (Jones, J Clin Pathol, 53:813-21 (2000)).

Other Autoimmune Diseases And Associated Self-Protein(s), -Polypeptide(s) Or -Peptide(s): Autoantigens for myasthenia gravis may include epitopes within the acetylcholine receptor. Autoantigens targeted in pemphigus vulgaris may include desmoglein-3. Sjogren's syndrome antigens may include SSA (Ro); SSB (La); and fodrin. The dominant autoantigen for pemphigus vulgaris may include desmoglein-3. Panels for myositis may include tRNA synthetases (e.g., threonyl, histidyl, alanyl, isoleucyl, and glycyl); Ku; Scl; SS-A; U1-sn-ribonuclear proteins; Mi-1; Mi-1; Jo-1; Ku; and SRP. Panels for scleroderma may include Scl-70; centromere; U1-sn-ribonuclear proteins; and fibrillarin. Panels for pernicious anemia may include intrinsic factor; and glycoprotein beta subunit of gastric H/K ATPase. Epitope Antigens for systemic lupus erythematosus (SLE) may include DNA; phospholipids; nuclear antigens; U1 ribonucleoprotein; Ro60 (SS-A); Ro52 (SS-A); La (SS-B); calreticulin; Grp78; Scl-70; histone; Sm protein; serine-arginine splicing factors, and chromatin, etc. For Grave's disease epitopes may include the Na+/I− symporter; thyrotropin receptor; Tg; and TPO.

Other diseases

Several examples of other diseases associated with self-antigen(s), -proteins(s), -polypeptide(s) or -peptide(s) present in the animal non-physiologically are set forth in the table and described below.

### Inflammatory Diseases

Osteoarthritis and Degenerative Joint Diseases: Osteoarthritis (OA) affects 30% of people over 60 years of age, and is the most common joint disease of humans. Osteoarthritis represents the degeneration and failure of synovial joints, and involves breakdown of the articular cartilage.

Cartilage is composed primarily of proteoglycans, which provide stiffness and ability to withstand load, and collagens that provide tensile and resistance to sheer strength. Chondrocytes turn over and remodel normal cartilage by producing and secreting latent collagenases, latent stromelysin, latent gelatinase, tissue plasminogen activator and other associated enzymes, each of which alone or in combination is a self-lipids, self-antigen(s), self-proteins(s), self-peptide(s), self-polypeptide(s), self-glycolipid(s), self-carbohydrate(s), self-glycoprotein(s), and posttranslationally-modified self- protein(s), peptide(s), polypeptide(s), or glycoprotein(s) of this invention. Several inhibitors, including tissue inhibitor of metalloproteinase (TIMP) and plasminogen activator inhibitor (PAI-1), are also produced by chondrocytes and limit the degradative activity of neutral metalloproteinases, tissue plasminogen activator, and other enzymes. These degradative enzymes and inhibitors, alone or in combination, are the self-antigen(s), self-proteins(s), polypeptide(s) or peptide(s) of this invention. These degradative enzymes and inhibitors coordinate remodeling and maintenance of normal cartilage. In OA, dysregulation of this process results in the deterioration and degradation of cartilage. Most patients with OA also have some degree of inflammation, including warmth and swelling of joints. In early OA there are abnormal alterations in the arrangement and size of collagen fibers. Metalloproteinases, cathepsins, plasmin, and other self molecules alone or in combination are self-lipids, self-antigen(s), self-proteins(s), self-peptide(s), self-polypeptide(s), self-glycolipid(s), self-carbohydrate(s), self-glycoprotein(s), and posttranslationally-modified self- protein(s), peptide(s), polypeptide(s), or glycoprotein(s) of this invention, cause significant cartilage matrix loss. Initially increased chondrocyte production of proteoglycans and cartilage results in the articular cartilage being thicker than normal. The articular cartilage then thins and softens as a result of the action of degradative enzymes including collagenases, stromelysin, gelatinase, tissue plasminogen activator and other related enzymes, alone or in combination are self molecules such as self-lipids, self-antigen(s), self-proteins(s), self-peptide(s), self-polypeptide(s), self-glycolipid(s), self-carbohydrate(s), self-glycoprotein(s), and posttranslationally-modified self- protein(s), peptide(s), polypeptide(s), or glycoprotein(s) of this invention. Inflammatory molecules such as IL-1, cathepsins, and plasmin may promote the degeneration and breakdown of cartilage, alone or in combination, and are self-lipids, self-antigen(s), self-proteins(s), self-peptide(s), self-polypeptide(s), self-glycolipid(s), self-carbohydrate(s), self-glycoprotein(s), and posttranslationally-modified self- protein(s), peptide(s), polypeptide(s), or glycoprotein(s) of this invention. The softer and thinner cartilage is much more susceptible to damage by mechanical stress. These factors lead to the breakdown of the cartilage surface and the formation of vertical clefts (fibrillation). Erosions in the cartilage surface form, and extend to bone in end-stage disease. Chondrocytes initially replicate and form clusters, and at end-stage the cartilage is hypocelluar. Remodeling and hypertrophy of bone are significant features of OA.

Current therapies for OA include rest, physical therapy to strengthen muscles supporting the joint, braces and other supportive devices to stabilize the joint, non-steroidal anti-inflammatory agents, acetaminophen, and other analgesics. In end-stage bone-on-bone OA of joints critical for activities of daily living, such as the knees or hips, surgical joint replacement is performed.

Spinal Cord Injury: It is estimated that there are approximately 11,000 new cases of spinal cord injury every year in the U.S. and that the overall prevalence is a total of 183,000 to 230,000 cases in the U.S. presently (Stover et al., Arch Phys Med Rehabil, 80, 1365-71,1999). Recovery from spinal cord injury is very poor and results in devastating irreversible neurologic disability. Current treatment of acute spinal cord injury consists of mechanical stabilization of the injury site, for example by surgical intervention, and the administration of parenteral steroids. These interventions have done little to reduce the incidence of permanent paralysis following spinal cord injury. Treatment of chronic spinal cord injury is focused on maintenance of quality of life such as the management of pain, spasticity, and bladder function. No currently available treatment addresses the recovery of neurologic function. In the acute stage immediately following injury, inflammation is prominent, and swelling associated with cord damage is a major cause of morbidity. This inflammation is controlled in part with high doses of systemic corticosteroids.

Graft Versus Host Disease: One of the greatest limitations of tissue and organ transplantation in humans is rejection of the tissue transplant by the recipient's immune system. It is well established that the greater the matching of the MHC class I and II (HLA-A, HLA-B, and HLA-DR) alleles between donor and recipient the better the graft survival. Graft versus host disease (GVHD) causes significant morbidity and mortality in patients receiving transplants containing allogeneic hematopoietic cells. This is due in part to inflammation in the skin and in other target organs. Hematopoietic cells are present in bone-marrow transplants, stem cell transplants, and other transplants. Approximately 50% of patients receiving a transplant from a HLA-matched sibling will develop moderate to severe GVHD, and the incidence is much higher in non-HLA-matched grafts. One-third of patients who develop moderate to severe GVHD will die as a result. T lymphocytes and other immune cell in the donor graft attack the recipients cells that express polypeptides variations in their amino acid sequences, particularly variations in proteins encoded in the major histocompatibility complex (MHC) gene complex on chromosome 6 in humans. The most influential proteins for GVHD in transplants involving allogeneic hematopoietic cells are the highly polymorphic (extensive amino acid variation between people) class I proteins (HLA-A, -B, and -C) and the class II proteins (DRB1, DQB1, and DPB1) (Appelbaum, Nature 411:385-389, 2001). Even when the MHC class I alleles are serologically ‘matched’ between donor and recipient, DNA sequencing reveals there are allele-level mismatches in 30% of cases providing a basis for class I-directed GVHD even in matched donor-recipient pairs (Appelbaum, Nature, 411, 385-389, 2001). GVHD frequently causes damage to the skin, intestine, liver, lung, and pancreas. GVHD is treated with glucocorticoids, cyclosporine, methotrexate, fludarabine, and OKT3.

Tissue Transplant Rejection: Immune rejection of tissue transplants, including lung, heart, liver, kidney, pancreas, and other organs and tissues, is mediated by immune responses in the transplant recipient directed against the transplanted organ. Allogeneic transplanted organs contain proteins with variations in their amino acid sequences when compared to the amino acid sequences of the transplant recipient. Because the amino acid sequences of the transplanted organ differ from those of the transplant recipient they frequently elicit an immune response in the recipient against the transplanted organ. The immune response encompasses responses by both the innate and the acquired immune system and is characterized by inflammation in the target organ. Rejection of transplanted organs is a major complication and limitation of tissue transplant, and can cause failure of the transplanted organ in the recipient. The chronic inflammation that results from rejection frequently leads to dysfunction in the transplanted organ. Transplant recipients are currently treated with a variety of immunosuppressive agents to prevent and suppress rejection. These agents include glucocorticoids, cyclosporin A, Cellcept, FK-506, and OKT3.

Immune Modulatory Nucleic Acids and Methods of Use In certain embodiments, the present invention provides a pharmaceutical composition comprising: (a) an immune modulatory nucleic acid comprising an immune modulatory sequence comprising: (i) a hexameric sequence 5′-Purine-Pyrimidine_{[1]}-[X]-[Y]-Pyrimidine_{[2]}-Pyrimidine_{[3]}-3′, wherein X and Y are any naturally occurring or synthetic nucleotide, except that X and Y cannot be cytosine-guanine, X and Y cannot be cytosine-cytosine when Pyrimidine_{[2]} is thymine, X and Y cannot be cytosine-thymine when Pyrimidine_{[1]} is cytosine, and the immune modulatory sequence does not contain cytosine-guanine sequences; (ii) a CC dinucleotide 5′ to the hexameric sequence, wherein the CC dinucleotide is positioned between one to five nucleotides 5′ of the hexameric sequence; and (iii) a polyG region 3′ of the hexameric sequence, wherein the polyG comprises at least three contiguous Gs and is positioned between two to five nucleotides 3′ of the hexameric sequence; and (b) a pharmaceutically acceptable carrier.

In certain embodiments, the pharmaceutical composition comprises: (a) an immune modulatory nucleic acid comprising an immune modulatory sequence comprising: (i) a hexameric sequence 5′-Purine-Pyrimidine_{[1]}-[X]-[Y]-Pyrimidine_{[2]}-Pyrimidine_{[3]}-3′, wherein X and Y are any naturally occurring or synthetic nucleotide, except that X and Y cannot be cytosine-guanine, X and Y cannot be cytosine-cytosine when Pyrimidine_{[2]} is thymine, X and Y cannot be cytosine-thymine when Pyrimidine_{[1]} is cytosine, and the immune modulatory sequence does not contain cytosine-guanine sequences; (ii) a CC dinucleotide 5′ to the hexameric sequence, wherein the CC dinucleotide is positioned two nucleotides 5′ of the hexameric sequence; and (iii) a polyG region 3′ of the hexameric sequence, wherein the polyG comprises at least three contiguous Gs and is positioned between two to five nucleotides 3′ of the hexameric sequence; and (b) a pharmaceutically acceptable carrier.

In certain embodiments, the pharmaceutical composition comprises: (a) an immune modulatory nucleic acid comprising an immune modulatory sequence comprising: (i) a hexameric sequence 5′-Purine-Pyrimidine_{[1]}-[X]-[Y]-Pyrimidine_{[2]}-Pyrimidine_{[3]}-3′, wherein X and Y are any naturally occurring or synthetic nucleotide, except that X and Y cannot be cytosine-guanine, X and Y cannot be cytosine-cytosine when Pyrimidine_{[2]} is thymine, X and Y cannot be cytosine-thymine when Pyrimidine_{[1]} is cytosine, and the immune modulatory sequence does not contain cytosine-guanine sequences; (ii) a CC dinucleotide 5′ to the hexameric sequence, wherein the CC dinucleotide is positioned between one to five nucleotides 5′ of the hexameric sequence; and (iii) a polyG region 3′ of the hexameric sequence, wherein the polyG region comprises at least three continugous Gs and is positioned two nucleotides 3′ of the hexameric sequence; and (b) a pharmaceutically acceptable carrier.

In certain embodiments, the pharmaceutical composition comprises: (a) an immune modulatory nucleic acid comprising an immune modulatory sequence comprising: (i) a hexameric sequence 5′-Purine-Pyrimidine_{[1]}-[X]-[Y]-Pyrimidine_{[2]}-Pyrimidine_{[3]}-3′, wherein X and Y are any naturally occurring or synthetic nucleotide, except that X and Y cannot be cytosine-guanine, X and Y cannot be cytosine-cytosine when Pyrimidine_{[2]} is thymine, X and Y cannot be cytosine-thymine when Pyrimidine_{[1]} is cytosine, and the immune modulatory sequence does not contain cytosine-guanine sequences; (ii) a CC dinucleotide 5′ to the hexameric sequence, wherein the CC dinucleotide is positioned two nucleotides 5′ of the hexameric sequence; and (iii) a polyG region 3′ of the hexameric sequence, wherein the polyG region comprises at least three contiguous Gs and is positioned two nucleotides 3′ of the hexameric sequence; and (b) a pharmaceutically acceptable carrier.

In certain embodiments, the pharmaceutical composition comprises: (a) an immune modulatory nucleic acid comprising an immune modulatory sequence comprising: (i) a hexameric sequence 5′-Purine-Pyrimidine_{[1]}-[X]-[Y]-Pyrimidine_{[2]}-Pyrimidine_{[3]}-3′, wherein X and Y of the hexameric sequence are guanine-guanine and the immune modulatory sequence does not contain cytosine-guanine sequences; (ii) a CC dinucleotide 5′ to the hexameric sequence, wherein the CC dinucleotide is positioned between one to five nucleotides 5′ of the hexameric sequence; and (iii) a polyG region 3′ of the hexameric sequence, wherein the polyG comprises at least three contiguous Gs and is positioned between two to five nucleotides 3′ of the hexameric sequence; and (b) a pharmaceutically acceptable carrier.

In certain embodiments, the pharmaceutical composition comprising: (a) an immune modulatory nucleic acid comprising an immune modulatory sequence comprising: (i) a hexameric sequence 5′-Purine-Pyrimidine_{[1]}-[X]-[Y]-Pyrimidine_{[2]}-Pyrimidine_{[3]}-3′, wherein X and Y are guanine-guanine and the immune modulatory sequence does not contain cytosine-guanine sequences; (ii) a CC dinucleotide 5′ to the hexameric sequence, wherein the CC dinucleotide is positioned two nucleotides 5′ of the hexameric sequence; and (iii) a polyG region 3′ of the hexameric sequence, wherein the polyG comprises at least three contiguous Gs and is positioned between two to five nucleotides 3′ of the hexameric sequence; and (b) a pharmaceutically acceptable carrier.

In certain embodiments, the pharmaceutical composition comprises: (a) an immune modulatory nucleic acid comprising an immune modulatory sequence comprising: (i) a hexameric sequence 5′-Purine-Pyrimidine_{[1]}-[X]-[Y]-Pyrimidine_{[2]}-Pyrimidine_{[3]}-3′, wherein X and Y are guanine-guanine and the immune modulatory sequence does not contain cytosine-guanine sequences; (ii) a CC dinucleotide 5′ to the hexameric sequence, wherein the CC dinucleotide is positioned between one to five nucleotides 5′ of the hexameric sequence; and (iii) a polyG region 3′ of the hexameric sequence, wherein the polyG comprises at least three contiguous Gs and is positioned two nucleotides 3′ of the hexameric sequence; and (b) a pharmaceutically acceptable carrier.

In certain embodiments, the pharmaceutical composition comprises: (a) an immune modulatory nucleic acid comprising an immune modulatory sequence comprising: (i) a hexameric sequence 5′-Purine-Pyrimidine_{[1]}[X]-[Y]-Pyrimidine_{[2]}-Pyrimidine_{[3]}-3′, wherein X and Y are guanine-guanine and the immune modulatory sequence does not contain cytosine-guanine sequences; (ii) a CC dinucleotide 5′ to the hexameric sequence, wherein the CC dinucleotide is positioned two nucleotides 5′ of the hexameric sequence; and (iii) a polyG region 3′ of the hexameric sequence, wherein the polyG comprises at least three contiguous Gs and is positioned two nucleotides 3′ of the hexameric sequence; and (b) a pharmaceutically acceptable carrier.

In certain embodiments, the pharmaceutical composition comprises: (a) an immune modulatory nucleic acid comprising an immune modulatory sequence comprising: (i) a hexameric sequence 5′-Purine-Pyrimidine_{[1]}-[X]-[Y]-Pyrimidine_{[2]}-Pyrimidine_{[3]}-3′, wherein the hexameric sequence is GTGGTT and the immune modulatory sequence does not contain cytosine-guanine sequences; (ii) a CC dinucleotide 5′ to the hexameric sequence, wherein the CC dinucleotide is positioned between one to five nucleotides 5′ of the hexameric sequence; and (iii) a polyG region 3′ of the hexameric sequence, wherein the polyG comprises at least three contiguous Gs and is positioned between two to five nucleotides 3′ of the hexameric sequence; and (b) a pharmaceutically acceptable carrier.

In certain embodiments, the pharmaceutical composition comprises: (a) an immune modulatory nucleic acid comprising an immune modulatory sequence comprising: (i) a hexameric sequence 5′-Purine-Pyrimidine_{[1]}[X]-[Y]-Pyrimidine_{[2]}-Pyrimidine_{[3]}-3′, wherein the hexameric sequence is GTGGTT and the immune modulatory sequence does not contain cytosine-guanine sequences; (ii) a CC dinucleotide 5′ to the hexameric sequence, wherein the CC dinucleotide is positioned two nucleotides 5′ of the hexameric sequence; and (iii) a polyG region 3′ of the hexameric sequence, wherein the polyG comprises at least three contiguous Gs and is positioned between two to five nucleotides 3′ of the hexameric sequence; and (b) a pharmaceutically acceptable carrier.

In certain embodiments, the pharmaceutical composition comprises: (a) an immune modulatory nucleic acid comprising an immune modulatory sequence comprising: (i) a hexameric sequence 5′-Purine-Pyrimidine_{[1]}-[X]-[Y]-Pyrimidine_{[2]}-Pyrimidine_{[3]}-3′, wherein the hexameric sequence is GTGGTT and the immune modulatory sequence does not contain cytosine-guanine sequences; (ii) a CC dinucleotide 5′ to the hexameric sequence, wherein the CC dinucleotide is positioned between one to five nucleotides 5′ of the hexameric sequence; and (iii) a polyG region 3′ of the hexameric sequence, wherein the polyG comprises at least three contiguous Gs and is positioned two nucleotides 3′ of the hexameric sequence; and (b) a pharmaceutically acceptable carrier.

In certain embodiments, the pharmaceutical composition comprises: (a) an immune modulatory nucleic acid comprising an immune modulatory sequence comprising: (i) a hexameric sequence 5′-Purine-Pyrimidine_{[1]}-[X]-[Y]-Pytimidine_{[2]}-Pyrimidine_{[3]}-3′, wherein the hexameric sequence is GTGGTT and the immune modulatory sequence does not contain cytosine-guanine sequences; (ii) a CC dinucleotide 5′ to the hexameric sequence, wherein the CC dinucleotide is positioned two nucleotides 5′ of the hexameric sequence; and (iii) a polyG region 3′ of the hexameric sequence, wherein the polyG comprises at least three contiguous Gs and is positioned two nucleotides 3′ of the hexameric sequence; and (b) a pharmaceutically acceptable carrier.

In certain embodiments, the pharmaceutical composition comprises: (a) an immune modulatory nucleic acid comprising an immune modulatory sequence wherein the immune modulatory sequence is CCATGTGGTTATGGGT; and (b) a pharmaceutically acceptable carrier. In certain embodiments, the pharmaceutical composition comprises an immune modultory nucleic acid of the present invention that is an oligonucleotide. In certain embodiments, the pharmaceutical composition comprises an immune modultory nucleic acid of the present invention that is incorporated into a vector. In certain embodiments, the pharmaceutical composition comprises an immune modultory nucleic acid of the present invention that is incorporated into an expression vector.

In certain embodiments, the present invention provides a method for treating a disease in a subject associated with one or more self-molecules present non-physiologically in the subject, the method comprising administering to the subject an immune modulatory sequence of the present invention. In certain embodiments, the present invention provides a method for treating a disease in a subject associated with one or more self-molecules present non-physiologically in the subject, the method comprising administering to the subject a pharmaceutical composition of the present invention. In certain embodiments, the present invention provides a method for treating systemic lupus erythematosus in a subject, the method comprising administering to the subject an immune modulatory sequence of the present invention. In certain embodiments, the present invention provides a method for treating systemic lupus erythematosus in a subject, the method comprising administering to the subject a pharmaceutical composition of the present invention.

In one aspect, the improved immune modulatory sequences of the present invention I.) comprise:

1.) a hexameric sequence
5′-Purine-Pyrimidine_{[1}]-[X]-[Y]-Pyrimidine_{[3]}-Pyrimidine_{[3]}-3′;

wherein X and Y are any naturally occurring or synthetic nucleotide, except that
a. X and Y cannot be cytosine-guanine;
b. that X and Y cannot be cytosine-cytosine when Pyrimidine_{[2]} is thymine
c. that X and Y cannot be cytosine-thymine when Pyrimidine_{[1]} is cytosine

2.) a CC dinucleotide 5′ to the hexameric sequence wherein the CC dinucleotide is positioned between one to five nucleotides 5′ of the hexameric sequence; and

3.) a polyG region 3′ of the hexameric sequence wherein the polyG comprises three contiguous Gs and is positioned between two to five nucleotides 3′ of the hexameric sequence

wherein the immune modulatory sequence does not contain cytosine-guanine sequences.

Alternatively, the improved immune modulatory sequences of the present invention comprise:

1.) a hexameric sequence
5′-Purine-Pyrimidine-[X]-[Y]-Pyrimidine-Pyrimidine-3′;
wherein X and Y are guanine-guanine;

2.) a CC dinucleotide 5′ to the hexameric sequence wherein the CC dinucleotide is positioned between one to five nucleotides 5′ of the hexameric sequence; and

3.) a polyG region 3′ of the hexameric sequence wherein the polyG comprises a) between two and ten contiguous Gs and b) are positioned between two to ten nucleotides 3′ of the hexameric sequence

wherein the immune modulatory sequence does not contain cytosine-guanine sequences.

In certain embodiments of the present invention, X and Y of the hexameric sequence are GpG. In certain embodiments the hexameric sequence is 5′-GTGGTT-3′. In certain embodiments the CC di-nucleotide is two nucleotides 5′ of the hexameric sequence. In certain embodiments the polyG region comprises three contiguous guanine bases and is positioned two nucleotides 3′ from the hexameric sequence. In certain embodiments the improved immune modulatory sequence is 5′-CCATGTGGTTATGGGT-3′.

The core hexamer of IMSs of the invention, referred to herein as the immune modulatory sequence motif comprising a dinucleotide motif, can be flanked 5′ and/or 3′ by any composition or number of nucleotides or nucleosides. In some embodiments, immune modulatory nucleic acids comprising one or more immune modulatory sequence are oligonucleotides ranging between 14 and 50, 75 and 100 base pairs in size, and most usually 15-50 base pairs in size. Immune modulatory nucleic acids can also be larger pieces of DNA, ranging from, for example, 100 to 100,000 base pairs and can be expression vectors and other plasmids, for example. Sequences present that flank the immunomodulatory sequence motif of the present invention can be constructed to substantially match flanking sequences present in any known immunoinhibitory sequences. For example, the IMS having the sequence TGACTGTG-CCNN-Purine-Pyrmidine -X-Y-Pyrimidine-Pyrimidine-NNGGG-AGAGATGA where N is any nucleotide, comprises the flanking sequences TGACTGTG and AGAGATGA. Another preferred flanking sequence incorporates a series of pyrimidines (C, T, and U), either as an individual pyrimidine repeated two or more times, or a mixture of different pyrimidines two or more in length. Different flanking sequences have been used in testing inhibitory modulatory sequences. Further examples of flanking sequences for inhibitory nucleic acids are contained in the following references: U.S. Pat. Nos. 6,225,292 and 6,339,068; Zeuner et al., Arthritis and Rheumatism, 46:2219-24, 2002.

Particular IMSs of the invention comprise the following hexamer sequences:
1. 5′-purine-pyrimidine-[X]-[Y]-pyrimidine-pyrimidine-3′ IMSs containing GG dinucleotide cores: GTGGTT, ATGGTT, GCGGTT, ACGGTT, GTGGCT, ATGGCT, GCGGCT, ACGGCT, GTGGTC, ATGGTC, GCGGTC, ACGGTC, and so forth;
2. 5′-purine-pyrimidine-[X]-[Y]-pyrimidine-pyrimidine-3′ IMSs containing GC dinucleotides cores: GTGCTT, ATGCTT, GCGCTT, ACGCTT, GTGCCT, ATGCCT, GCGCCT, ACGCCT, GTGCTC, ATGCTC, GCGCTC, ACGCTC, and so forth;
3. Guanine and inosine substitues for adenine and/or uridine substitutes for cytosine or thymine and those substitutions can be made as set forth based on the guidelines above.

A previously disclosed immune inhibitory sequence or IIS, was shown to inhibit immunostimulatory sequences (ISS) activity containing a core dinucleotide, CpG. U.S. Pat. No. 6,225,292. This IIS, in the absence of an ISS, was shown in WO 04/047734 to prevent and treat autoimmune disease either alone or in combination with DNA polynucleotide therapy. This IIS contained the core hexamer region having the sequence AAGGTT. Other related IISs with a similar motif included within the IMSs of this invention are:
1. 5′-purine-purine-[X]-[Y]-pyrimidine-pyrimidine-3′ IMSs containing GG dinucleotide cores: GGGGTT, AGGGTT, GAGGTT, AAGGTT, GGGGCT, AGGGCT, GAGGCT, AAGGCT, GGGGTC, AGGGTC, GAGGTC, AAGGTC, and so forth;
2. 5′-purine-purine-[X]-[Y]-pyrimidine-pyrimidine-3′ IMSs containing GC dinucleotide cores: GGGCTT, AGGCTT, GAGCTT, AAGCTT, GGGCCT, AGGCCT, GAGCCT, AAGCCT, GGGCTC, AGGCTC, GAGCTC, AAGCTC, and so forth;
3. Guanine and inosine substitutions for adenine and/or uridine substitutions for cytosine or thymine can be made as set forth based on the guidelines above.

In certain embodiments of the present invention, the core hexamer region of the IMS is flanked at either the 5′ or 3′ end, or at both the 5′ and 3′ ends, by a polyG region. A “polyG region” or “polyG motif” as used herein means a nucleic acid region consisting of at least two (2) contiguous guanine bases, typically from 2 to 30 or from 2 to 20 contiguous guanines. In some embodiments, the polyG region has from 2 to 10, from 4 to 10, or from 4 to 8 contiguous guanine bases. In certain embodiments, the flanking polyG region is adjacent to (i.e., abuts) the core hexamer. In certain embodiments, the polyG region is linked to the core hexamer by a non-polyG region (non-polyG linker). In some embodiments, the non-polyG linker region has no more than 6, more typically no more than 4 nucleotides, and most typically no more than 2 nucleotides.

In certain embodiments of the present invention, the core hexamer region of the IMS is flanked at either the 5′ or 3′ end, or at both the 5′ and 3′ ends, by a CC dinucleotide region. A “CC dinucleotide region” or “CC dinucleotide motif” as used herein means a nucleic acid region comprising 2 contiguous cytosine bases. In some embodiments, the CC dinucleotide region is 2, 3, 4, 5, 6, 7, 8, 9 or 10 nucleotide bases in length, but can be longer. In certain embodiments, the flanking CC dinucleotide is adjacent to (i.e., abuts) the core hexamer. In certain embodiments, the CC dinucleotide is linked to the core hexamer by a non-CC dinucleotide region (non-CC dinucleotide linker). In some embodiments, the non-CC dinucleotide linker region has about 8, 7, 6, 5, 4, 3 or 2 nucleotides.

Immune modulatory nucleic acids can be obtained from existing nucleic acid sources, including genomic DNA, plasmid DNA, viral DNA and cDNA. In certain embodiments, the immune modulatory nucleic acids are synthetic oligonucleotides produced by oligonucleotide synthesis. IMS can be part of single-strand or double-stranded DNA, RNA and/or oligonucleosides.

Immune modulatory nucleic acids are preferentially nucleic acids having one or more IMS regions that contain unmethylated GpG oligonucleotides. In alternative embodiments, one or more adenine or cytosine residues of the IMS region are methylated. In eukaryotic cells, typically cytosine and adenine residues can be methylated.

Immune modulatory nucleic acids can be stabilized and/or unstabilized oligonucleotides. Stabilized oligonucleotides mean oligonucleotides that are relatively resistant to in vivo degradation by exonucleases, endonucleases and other degradation pathways. Preferred stabilized oligonucleotides have modified phophate backbones, and most preferred oligonucleotides have phophorothioate modified phosphate backbones in which at least one of the phosphate oxygens is replaced by sulfur. Backbone phosphate group modifications, including methylphosphonate, phosphorothioate, phophoroamidate and phosphorodithionate internucleotide linkages, can provide antimicrobial properties on IMSs. The immune modulatory nucleic acids are preferably stabilized oligonucleotides, preferentially using phosphorothioate stabilized oligonucleotides.

Alternative stabilized oligonucleotides include: alkylphosphotriesters and phosphodiesters, in which the charged oxygen is alkylated; arylphosphonates and alkylphosphonates, which are nonionic DNA analogs in which the charged phosphonate oxygen is replaced by an aryl or alkyl group; or/and oligonucleotides containing hexaethyleneglycol or tetraethyleneglycol, or another diol, at either or both termini. Alternative steric configurations can be used to attach sugar moieties to nucleoside bases in IMS regions.

The nucleotide bases of the IMS region which flank the modulating dinucleotides may be the known naturally occurring bases or synthetic non-natural bases. Oligonucleosides may be incorporated into the internal region and/or termini of the IMS-ON using conventional techniques for use as attachment points, that is as a means of attaching or linking other molecules, for other compounds, including self-molecules or as attachment points for additional immune modulatory therapeutics. The base(s), sugar moiety, phosphate groups and termini of the IMS-ON may also be modified in any manner known to those of ordinary skill in the art to construct an IMS-ON having properties desired in addition to the modulatory activity of the IMS-ON. For example, sugar moieties may be attached to nucleotide bases of IMS-ON in any steric configuration.

The techniques for making these phosphate group modifications to oligonucleotides are known in the art and do not require detailed explanation. For review of one such useful technique, the intermediate phosphate triester for the target oligonucleotide product is prepared and oxidized to the naturally occurring phosphate triester with aqueous iodine or with other agents, such as anhydrous amines. The resulting oligonucleotide phosphoramidates can be treated with sulfur to yield phophorothioates. The same general technique (excepting the sulfur treatment step) can be applied to yield methylphosphoamidites from methylphosphonates. For more details concerning phosphate group modification techniques, those of ordinary skill in the art may wish to consult U.S. Pat. Nos. 4,425,732; 4,458,066; 5,218,103 and 5,453,496, as well as Tetrahedron, Lett. at 21:4149 25 (1995), 7:5575 (1986), 25:1437 (1984) and Journal Am. ChemSoc., 93:6657 (1987), the disclosures of which are incorporated herein for the purpose of illustrating the level of knowledge in the art concerning the composition and preparation of immune modulatory nucleic acids.

A particularly useful phosphate group modification is the conversion to the phosphorothioate or phosphorodithioate forms of the IMS-ON oligonucleotides. Phosphorothioates and phosphorodithioates are more resistant to degradation in vivo than their unmodified oligonucleotide counterparts, making the IMS-ON of the invention more available to the host.

IMS-ON can be synthesized using techniques and nucleic acid synthesis equipment which are well-known in the art. For reference in this regard, see, e.g., Ausubel, et al., Current Protocols in Molecular Biology, Chs. 2 and 4 (Wiley Interscience, 1989); Maniatis, et al., Molecular Cloning: A Laboratory Manual (Cold Spring Harbor Lab., New York, 1982); U.S. Pat. No. 4,458,066 and U.S. Pat. No. 4,650,675. These references are incorporated herein by reference for the purpose of demonstrating the level of knowledge in the art concerning production of synthetic oligonucleotides.

Alternatively, IMS-ON can be obtained by mutation of isolated microbial ISS-ODN to substitute a competing dinucleotide for the naturally occurring CpG motif and the flanking nucleotides. Screening procedures which rely on nucleic acid hybridization make it possible to isolate any polynucleotide sequence from any organism, provided the appropriate probe or antibody is available. Oligonucleotide probes, which correspond to a part of the sequence encoding the protein in question, can be synthesized chemically. This requires that short, oligo-peptide stretches of amino acid sequence must be known. The DNA sequence encoding the protein can also be deduced from the genetic code, however, the degeneracy of the code must be taken into account.

For example, a cDNA library believed to contain an ISS-containing polynucleotide can be screened by injecting various mRNA derived from cDNAs into oocytes, allowing sufficient time for expression of the cDNA gene products to occur, and testing for the presence of the desired cDNA expression product, for example, by using antibody specific for a peptide encoded by the polynucleotide of interest or by using probes for the repeat motifs and a tissue expression pattern characteristic of a peptide encoded by the polynucelotide of interest. Alternatively, a cDNA library can be screened indirectly for expression of peptides of interest having at least one epitope using antibodies specific for the peptides. Such antibodies can be either polyclonally or monoclonally derived and used to detect expression product indicative of the presence of cDNA of interest.

Once the ISS-containing polynucleotide has been obtained, it can be shortened to the desired length by, for example, enzymatic digestion using conventional techniques. The CpG motif in the ISS-ODN oligonucleotide product is then mutated to substitute an “inhibiting” dinucleotide—identified using the methods of this invention- for the CpG motif. Techniques for making substitution mutations at particular sites in DNA having a known sequence are well known, for example M13 primer mutagenesis through PCR. Because the IMS is non-coding, there is no concern about maintaining an open reading frame in making the substitution mutation. However, for in vivo use, the polynucleotide starting material, ISS-ODN oligonucleotide intermediate or IMS mutation product should be rendered substantially pure (i.e., as free of naturally occurring contaminants and LP S as is possible using available techniques known to and chosen by one of ordinary skill in the art).

The immune modulatory nucleic acids of the present invention can contain IMSs alone or incorporated in cis or in trans with other nucleic acid regions such as, for example, into a recombinant self-vector (plasmid, cosmid, virus or retrovirus) which may in turn code for any self- protein(s), -polypeptide(s), or -peptide(s) deliverable by a recombinant expression vector. In certain embodiments, the IMSs are administered without incorporation into a vector. In certain embodiments, the IMSs are incorporated into a vector such as, for example, an expression vector, which may be accomplished, for example, using conventional techniques as known to one of ordinary skill in the art (see, e.g., Ausubel, Current Protocols in Molecular Biology, supra).

For example, construction of recombinant expression vectors employs standard ligation techniques. For analysis to confirm correct sequences in vectors constructed, the ligation mixtures may be used to transform a host cell and successful transformants selected by antibiotic resistance where appropriate. Vectors from the transformants are prepared, analyzed by restriction and/or sequenced by, for example, the method of Messing, et al., Nucleic Acids Res., 9:309, 1981, the method of Maxam, et al., Methods in Enzymology, 65:499, 1980, or other suitable methods which will be known to those skilled in the art. Size separation of cleaved fragments is performed using conventional gel electrophoresis as described, for example, by Maniatis, et al., Molecular Cloning, pp. 133-134, 1982.

Host cells may be transformed with the expression vectors of this invention and cultured in conventional nutrient media modified as is appropriate for inducing promoters, selecting transformants or amplifying genes. The culture conditions, such as temperature, pH and the like are those previously used with the host cell selected for expression, and will be apparent to the ordinarily skilled artisan.

If a recombinant vector is utilized as a carrier for the IMS-ON of the invention, plasmids and cosmids are particularly preferred for their lack of pathogenicity. However, plasmids and cosmids are subject to degradation in vivo more quickly than viruses and therefore may not deliver an adequate dosage of IMS-ON to prevent or treat an inflammatory or autoimmune disease.

In a related aspect, a nucleic acid vector is provided in which a non-CpG dinucleotide is substituted for one or more CpG dinucleotides of the formula 5′-purine-pyrimidine-C-G-pyrimidine-pyrimidine-3′ or 5′-purine-purine-C-G-pyrimidine-pyrimidine-3′, thereby producing a vector in which IIS-associated immunostimulatory activity is reduced. Such vectors are useful, for example, in methods for administering immune modulatory nucleic acids and/or for administering a self vector encoding one or more self-antigen(s), -proteins(s), -polypeptides(s), or -peptide(s). For example, the cytosine of the CpG dinucleotide can be substituted with guanine, thereby yielding an IMS region having a GpG motif of the formula 5′-purine-pyrimidine-G-G-pyrimidine-pyrimidine-3′ or 5′-purine-purine-G-G-pyrimidine-pyrimidine-3′. The cytosine can also be substituted with any other non-cytosine nucleotide. The substitution can be accomplished, for example, using site-directed mutagenesis. Typically, the substituted CpG motifs are those CpGs that are not located in important control regions of the vector (e.g., promoter regions). In addition, where the CpG is located within a coding region of an expression vector, the non-cytosine substitution is typically selected to yield a silent mutation or a codon corresponding to a conservative substitution of the encoded amino acid.

For example, in certain embodiments, a modified pVAX1 vector is provided in which one or more CpG dinucleotides of the formula 5′-purine-pyrimidine-C-G-pyrimidine-pyrimidine-3′ is mutated by substituting the cytosine of the CpG dinucleotide with a non-cytosine nucleotide. The pVAX1 vector is known in the art and is commercially available from Invitrogen (Carlsbad, Calif.). In one exemplary embodiment, the modified pVAX1 vector has the following cytosine to non-cytosine substitutions within a CpG motif:

cytosine to guanine at nucleotides 784, 1161, 1218, and 1966;

cytosine to adenine at nucleotides 1264, 1337, 1829, 1874, 1940, and 1997; and

cytosine to thymine at nucleotides 1963 and 1987;

with additional cytosine to guanine mutations at nucleotides 1831, 1876, 1942, and 1999. (The nucleotide number designations as set forth above are according to the numbering system for pVAX1 provided by Invitrogen.) (See Example 3, infra.)

In some embodiments of the methods and compositions, a plurality of (i.e., two or more) immune inhibitory sequences, as described herein, are used. The plurality of IMS or IIS molecules can be administed or formulated separately or linked together, e.g., in tandem or in succession. The two or more immune inhibitory sequences can be the same or different sequences and can be linked together on the same molecule. In one embodiment, the IMS or IIS comprises two or more M49 sequences. In one embodiment, the IMS or IIS comprises two or more I18 sequences.

### Functional Properties of IMSs

There are several mechanisms to explain the immunomodulatory properties of IMSs, and these include mechanisms independent of ISS (CpG)-mediated immune stimulation.

“Modulation of, modulating or altering an immune response” as used herein refers to any alteration of existing or potential immune response(s) against self-molecules, including but not limited to nucleic acids, lipids, phospholipids, carbohydrates, self-antigen(s), -proteins(s), -polypeptide(s), -peptide(s), protein complexes, ribonucleoprotein complexes, or derivative(s) thereof that occurs as a result of administration of an immune modulatory nucleic acid. Such modulation includes any alteration in presence, capacity or function of any immune cell involved in or capable of being involved in an immune response. Immune cells include B cells, T cells, NK cells, NK T cells, professional antigen-presenting cells, non-professional antigen-presenting cells, inflammatory cells, or any other cell capable of being involved in or influencing an immune response. Modulation includes any change imparted on an existing immune response, a developing immune response, a potential immune response, or the capacity to induce, regulate, influence, or respond to an immune response. Modulation includes any alteration in the expression and/or function of genes, proteins and/or other molecules in immune cells as part of an immune response.

Modulation of an immune response includes, but is not limited to: elimination, deletion, or sequestration of immune cells; induction or generation of immune cells that can modulate the functional capacity of other cells such as autoreactive lymphocytes, APCs, or inflammatory cells; induction of an unresponsive state in immune cells, termed anergy; increasing, decreasing or changing the activity or function of immune cells or the capacity to do so, including but not limited to altering the pattern of proteins expressed by these cells. Examples include altered production and/or secretion of certain classes of molecules such as cytokines, chemokines, growth factors, transcription factors, kinases, costimulatory molecules, or other cell surface receptors; or any combination of these modulatory events.

The immune responses are characterized by helper T cells and immune responses that produce cytokines including IL-12 and IFN gamma, and are associated with B cells that produce antibodies of certain isotypes (generally, IgG2a in mice; generally, IgG1 and IgG3 in humans). Th1-type immune responses predominate in autoimmune diseases, and are associated with autoimmune-mediated tissue injury. In contrast, Th2 immune responses are characterized by helper T cells and immune responses that produce cytokines including IL-4 and IL-10, and are associated with B cells that produce antibodies of certain isotypes (generally, IgG1 in mice; generally, IgG2 and IgG4 in humans). Th2-type immune responses are associated with protection against autoimmune-mediated tissue injury in rodent and human autoimmunity.

Immune modulatory nucleic acids could modulate immune responses by eliminating, sequestering, or turning-off immune cells mediating or capable of mediating an undesired immune response; inducing, generating, or turning on immune cells that mediate or are capable of mediating a protective immune response; changing the physical or functional properties of immune cells (such as suppressing a Th1-type response and/or inducing a Th2-type response); or a combination of these effects. Examples of measurements of the modulation of an immune response include, but are not limited to, examination of the presence or absence of immune cell populations (using flow cytometry, immunohistochemistry, histology, electron microscopy, the polymerase chain reaction); measurement of the functional capacity of immune cells including ability or resistance to proliferate or divide in response to a signal (such as using T cell proliferation assays and pepscan analysis based on 3H-thymidine incorporation following stimulation with anti-CD3 antibody, anti-T cell receptor antibody, anti-CD28 antibody, calcium ionophores, PMA, antigen presenting cells loaded with a peptide or protein antigen; B cell proliferation assays); measurement of the ability to kill or lyse other cells (such as cytotoxic T cell assays); measurements of the cytokines, chemokines, cell surface molecules, antibodies and other products of the cells (by flow cytometry, enzyme-linked immunosorbent assays, Western blot analysis, protein microarray analysis, immunoprecipitation analysis); measurement of biochemical markers of activation of immune cells or signaling pathways within immune cells (Western blot and immunoprecipitation analysis of tyrosine, serine or threonine phosphorylation, polypeptide cleavage, and formation or dissociation of protein complexes; protein array analysis; DNA transcriptional profiling using DNA arrays or subtractive hybridization); measurements of cell death by apoptosis, necrosis, or other mechanisms (annexin V staining, TUNEL assays, gel electrophoresis to measure DNA laddering, histology; fluorogenic caspase assays, Western blot analysis of caspase substrates); measurement of the genes, proteins, and other molecules produced by immune cells (Northern blot analysis, polymerase chain reaction, DNA microarrays, protein microarrays, 2-dimentional gel electrophoresis, Western blot analysis, enzyme linked immunosorbent assays, flow cytometry); and measurement of clinical outcomes such as improvement of autoimmune, neurodegenerative, and other disease outcomes (clinical scores, requirements for use of additional therapies, functional status, imaging studies).

Other investigators have carried out experiments to evaluate the mechanisms of action of IISs. Those investigators demonstrated that neutralizing or suppressive IISs (GpGs) motifs, block ISS (CpG) immune stimulation (Krieg et al., PNAS, 95:12631, 1998; U.S. Pat. Nos. 6,225,292 and 6,339,068). The IISs in those experiments were used to counteract, inhibit, compete, or overcome the effects of ISSs (from such sources such as bacteria, viruses, parasites, and DNA given exogenously such as in DNA vaccination or gene therapy). ISSs and IISs have been shown to enter the same cell, suggesting that one mechanism by which IISs inibit ISSs is through direct competion within the same cell (Yamada et al., J. Immunology, 2002, 169:5590).

### Methods of Administration

The immune modulatory nucleic acids are prepared as a composition comprising a pharmaceutically acceptable carrier. Pharmaceutically acceptable carriers preferred for use with the immune modulatory nucleic acid of the invention may include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like. A composition of immune modulatory nucleic acids may also be lyophilized using means well known in the art, for subsequent reconstitution and use according to the invention. Immune modulatory nucleic acids can be mixed into a pharmaceutical composition that contain multiple copies of an individual IMS, a combination of different IMSs, a combination of IMSs where each is present at the same relative molar concentration, a combinations of IMSs where each is present at different relative molar concentrations, or individual and/or different IMSs incorporated into recombinant expression vector plasmids, linear polynucleotides, viruses and viral vectors, bacteria, and other live, inactivated or synthetic compositions containing oligonucleotides.

The immune modulatory nucleic acids of this invention can be formulated with salts for use as pharmaceuticals. Immune modulatory nucleic acids can be prepared with non-toxic inorganic or organic bases. Inorganic base salts include sodium, potassium, zinc, calcium, aluminum, magnesium, etc. Organic non-toxic bases include salts of primary, secondary and tertiary amines, and the like. Such immune modulatory nucleic acids can be formulated in lyophilized form for reconstitution prior to delivery, such as sterile water or a salt solution. Alternatively, immune modulatory nucleic acids can be formulated in solutions, suspensions, or emulsions involving water- or oil-based vehicles for delivery. Immune modulatory nucleic acids can be lyophilized and then reconstituted with sterile water prior to administration.

As known to those ordinarily skilled in the art, a wide variety of methods exist to deliver nucleic acids to subjects. In some embodiments, the immune modulatory nucleic acid is administered as a naked nucleic acid. For example, in certain embodiments, viral particles (e.g., adenovirus particles, see, e.g., Curiel et al., Am. J. Respir. Cell Mol. Biol., 6:247-52, 1992, supra) are mixed with the naked nucleic acid prior to administration to produce a formulation that contains viral particles not encapsulating the nucleic acid but which still facilitate its delivery. In certain embodiments, the immune modulatory nucleic acid is encapsulated or is complexed with molecule that binds to the nucleic acid such as, for example, cationic substances (e.g., DEAE-dextran or cationic lipids). For example, liposomes represent effective means to formulate and deliver oligonucleotdie and/or self-polynucleotide. See, Pack, et al. (2005) “Design and Development of Polymers for Gene Delivery” Nature Drug Discovery 4:581-493. In certain embodiments, the immune modulatory nucleic acid is incorporated into a viral vector, viral particle, or bacterium for pharmacologic delivery. Viral vectors can be infection competent, attenuated (with mutations that reduce capacity to induce disease), or replication-deficient. In some embodiments, the nucleic acid is conjugated to solid supports including gold particles, polysaccharide-based supports, or other particles or beads that can be injected, inhaled, or delivered by particle bombardment (ballistic delivery).

Methods for delivering nucleic acid preparations are known in the art. See, e.g., U.S. Pat. Nos. 5,399,346, 5,580,859, 5,589,466. A number of viral based systems have been developed for transfer into mammalian cells. For example, retroviral systems have been described (U.S. Pat. No. 5,219,740; (Miller et al., Biotechniques, 7:980-990, 1989; Miller, A. D., Human Gene Therapy, 1:5-14, 1990; Scarpa et al., Virology, 180:849-852, 1991; Burns et al., Proc. Natl. Acad. Sci. USA, 90:8033-8037, 1993); and (Boris-Lawrie and Temin, Cur. Opin. Genet. Develop., 3:102-109, 1993). A number of adenovirus vectors have also been described, see, e.g., (Haj-Ahmad et al., J. Virol., 57:267-274, 1986; Bett et al., J. Virol., 67:5911-5921, 1993; Mittereder et al., Human Gene Therapy, 5:717-729, 1994; Seth et al., J. Virol., 68:933-940, 1994; Barr et al., Gene Therapy, 1:51-58, 1994; Berkner, K. L., BioTechniques, 6:616-629, 1988); and (Rich et al., Human Gene Therapy, 4:461-476, 1993). Adeno-associated virus (AAV) vector systems have also been developed for nucleic acid delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993; Lebkowski et al., Molec. Cell. Biol,. 8:3988-3996, 1988; Vincent et al., Vaccines , 90 (Cold Spring Harbor Laboratory Press) 1990; Carter, B. J., Current Opinion in Biotechnology, 3:533-539, 1992; Muzyczka, N., Current Topics in Microbiol. And Immunol., 158:97-129, 1992; Kotin, R. M., Human Gene Therapy, 5:793-801, 1994); Shelling et al., Gene Therapy, 1:165-169, 1994); and Zhou et al., J. Exp. Med., 179:1867-1875, 1994).

The IMSs of this invention can also be delivered without a vector. For example, the molecule can be packaged in liposomes prior to delivery to the subject. Lipid encapsulation is generally accomplished using liposomes that are able to stably bind or entrap and retain nucleic acid. For a review of the use of liposomes as carriers for delivery of nucleic acids, see, (Hug et al., Biochim. Biophys. Acta., 1097:1-17, 1991); Straubinger et al., in Methods of Enzymology, Vol. 101, pp. 512-527, 1983). For example, lipids that can be used in accordance with the invention include, but are not limited to, DOPE (Dioleoyl phosphatidylethanolamine), cholesterol, and CUDMEDA (N-(5-cholestrum-3-ol 3 urethanyl)-N′,N′-dimethylethylenediamine). As an example, DNA can be administered in a solution containing one of the following cationic liposome formulations: Lipofectin™ (LTI/BRL), Transfast™ (Promega Corp), Tfx50™ (Promega Corp), Tfx10™ (Promega Corp), or Tfx20™ (Promega Corp). See also, Pack, et al. (2005) “Design and Development of Polymers for Gene Delivery” Nature Drug Discovery 4:581-493.

“Therapeutically effective amounts” of the immune modulatory nucleic acids are administered in accord with the teaching of this invention and will be sufficient to treat or prevent the disease as for example by ameliorating or eliminating symptoms and/or the cause of the disease. For example, therapeutically effective amounts fall within broad range(s) and are determined through clinical trials and for a particular patient is determined based upon factors known to the ordinarily skilled clinician including the severity of the disease, weight of the patient, age and other factors. Therapeutically effective amounts of immune modulatory nucleic acids are in the range of about 0.001 micrograms to about 1 gram. A preferred therapeutic amount of immune modulatory nucleic acid is in the range of about 5 micrograms to about 1000 micrograms of each. A most preferred therapeutic amount of an immune modulatory nucleic acid is in the range of about 50 to 200 micrograms. Immune modulatory nucleic acid therapy is delivered daily, every-other-day, twice-per-week, weekly, every-two-weeks or monthly on an ongoing basis. If delivered in conjunction with polynucleotide therapies encoding self-proteins, -polypeptides, or -peptides then the therapeutic regimen may be administered for various periods such as 6-12 months, and then every 3-12 months as a maintenance dose. Alternative treatment regimens may be developed depending upon the severity of the disease, the age of the patient, the oligonucleotide and/or polynucleotide encoding self-antigen(s), -proteins(s), -polypeptide(s) or -peptide(s) being administered and such other factors as would be considered by the ordinary treating physician.

In certain embodiments the immune modulatory nucleic acids are delivered by intramuscular injection. In certain embodiments the immune modulatory nucleic acids are delivered intranasally, orally, subcutaneously, intradermally, intravenously, impressed through the skin, intraocularly, intraarticularly, intravaginally, intrarectally, mucosally, or attached to gold particles delivered to or through the dermis (see, e.g., WO 97/46253). Alternatively, nucleic acid can be delivered into skin cells by topical application with or without liposomes or charged lipids (see, e.g, U.S. Pat. No. 6,087,341). Yet another alternative is to deliver the nucleic acid as an inhaled agent. In the case of combination therapy comprising the administration of immune modulatory nucleic acids and polynucleotides encoding a self-antigen(s), -proteins(s), -polypeptide(s), or -peptide(s), the immune modulatory nucleic acid and the polynucleotide can be administered at the same site, or at different sites, as well as at the same time, or at different times.

Prior to delivery of immune modulatory nucleic acids, the delivery site can be preconditioned by treatment with bupivicane, cardiotoxin or another agent that may enhance the delivery of subsequent polynucleotide therapy. Such preconditioning regimens are generally delivered 12 to 96 hours prior to delivery of therapeutic polynucleotide, more frequently 24 to 48 hours prior to delivery of the therapeutic immune modulatory nucleic acids. Alternatively, no preconditioning treatment is given prior to IMS therapy.

The immune modulatory nucleic acids and/or self-vector comprising a polynucleotide encoding the self-antigen(s), -proteins(s), -polypeptide(s), or -peptide(s) can be administered in combination with other substances, such as pharmacological agents, adjuvants, cytokines, self-lipids, self-antigen(s), self-proteins(s), self-peptide(s), self-polypeptide(s), self-glycolipid(s), self-carbohydrate(s), self-glycoprotein(s), and posttranslationally-modified self- protein(s), peptide(s), polypeptide(s), glycoprotein(s), DNA-based therapies, or in conjunction with delivery of vectors encoding cytokines.

In certain embodiments of the present invention the immune modulatory nucleic acids are administered in combination with other therapies. Such therapies could include, for example, immune modulatory nucleic acids administered in combination with self-molecules including, but not limited to, DNA encoding self molecules as described in Table 1, for example in the case of polynucleotide therapy (see US Patent Application Publication 20030148983), or with self-lipids, self-antigen(s), self-proteins(s), self-peptide(s), self-polypeptide(s), self-glycolipid(s), self-carbohydrate(s), self-glycoprotein(s), and posttranslationally-modified self- protein(s), peptide(s), polypeptide(s), or glycoprotein(s), or any other therapeutic compound used to treat autoimmune disease. In certain embodiments, the immune modulatory nucleic acids are administered to a patient with SLE in combination with polynucleotide therapy using one or more of the self-molecules associated with SLE as described in Table 1. In certain embodiments, the immune modulatory nucleic acids of the present invention are administered to a patient with SLE in combination with a medication used in the treatment of lupus including, but not limited to, non-steroidal anti-inflammatory drugs (NAIDS); antimalarials; corticosteroids; cytotoxics and immunosuppressants. In certain embodiments the immune modulatory nucleic acid administered to a patient with SLE is I18. In certain embodiments, the immune modulatory nucleic acids are administered to a patient with multiple sclerosis in combination with polynucleotide therapy using one or more of the self-molecules associated with multiple sclerosis as described in Table 1. In some embodiments, the immune modulatory nucleic acids are administered to a patient with multiple sclerosis in combination with a medication used in the treatment of multiple sclerosis including, but not limited to, alpha-interferon, beta-interferon and Copaxone. In certain embodiments the immune modulatory nucleic acid administered to a patient with multiple sclerosis is I18. In certain embodiments, the immune modulatory nucleic acids are administered to a patient with insulin dependent diabetes mellitus in combination with polynucleotide therapy using one or more of the self-molecules associated with insulin dependent diabetes mellitus as described in Table 1. In certain embodiments the immune modulatory nucleic acid administered to a patient with insulin dependent diabetes mellitus is I18.

A further understanding of the present invention will be obtained by reference to the following description that sets forth illustrative embodiments.

### Example 1

### IMS Inhibit CpG-ODN Induced Cell Proliferation and Cytokine Production in Human Peripheral Blood Mononuclear Cells (hPBMC)

A series of experiments were conducted to demonstrate that IMS can inhibit PBMC responses to CpG containing oligonucleotides (CpG-ODN). Stimulatory CpG-ODNs are known to act directly on human B cells and plasmacytoid dendritic cells (pDC) stimulating proliferation and secretion of IL-6 and IL-10 in B cells and the production of IFN-alpha by pDCs (Hartmann et al., PNAS 96:9305; Krug et al., Eur. J. Immunol. 31:2154; Vollmer et al., Eur. J. Immunol. 34:251; Fearon et al., Eur. J. Immunol. 33:2114; Marshall et al., J. Leuk. Biol. 73:781; Hartmann et al., Eur. J. Immunol. 33:1633). In addition, in PBMC cultures, “bystander” cells (monocytes, NK cells, macrophages) may respond to the cytokines produced by B and pDC cells and produce additional immune regulators (Hornung et al., J. Immunol. 168:4531; Krug et al., Eur. J. Immunol. 31:2154; Krieg, Ann. Rev. Immunol. 20:709; Kranzer, Immunol. 99:170).

A panel of IMS listed in Table 2 were synthesized and tested for the ability to inhibit these CpG-ODN stimulated responses. All the IMS contained at least one copy of the core “RYGGYY” motif but varied both in the length (˜14-42 bases) and in sequence identity of the bases flanking this core motif. Some oligos contained poly G sequences with the potential of forming oligonucleotide multimers or G-quadruplexes (Gursel et al., J. Immunol. 171:1393; Petraccone et al., International J. Biol. Macromolecules 31:131; Wu et al., J. Biol. Chem. 279:33071; Lee et al., NAR 8:4305; Phillips et al., J. Mol. Biol. 273:171). Most oligos had fully phosphorothioated backbones while others were partially phosphorothioated possessing a few modified bases at the 5′ and 3′ ends of the oligo as indicated in Table 2.

TABLE 2

| | |
|---|---|
| IMS ID | IMS Sequence |

RYGGYY Class

| | |
|---|---|
| I1 | T*C*C*A*T*G*T*G*G*T*T*C*C*T*G*A*C*C*A*T* |
| I5 | G*G*T*G*C*A*T*G*G*T*T*G*C*A*G* |
| I6 | T*G*G*T*G*G*T*T*T*T*G*G*C*C*T*T*T*T*G*G*C*C* |
| I7 | T*G*A*C*T*G*T*G*G*T*G*G*C*C*A*C*A*G*A*T*G*A* |
| I19 | C*C*A*T*G*T*G*G*T*T*A*T*T*T*T* |
| I20 | C*T*G*T*G*G*T*G*G*T*T*A*G*A*G*A* |
| I18.5 | C*C*G*T*G*G*T*T*A*T*G*G*T* |
| I18.13 | C*C*T*G*T*G*G*C*C*A*T*G*G*T* |
| I18.17 | C*C*A*T*G*T*G*G*T*T*A*T*G*G*T* |
| I18.18 | C*C*A*A*G*T*G*G*T*T*A*T*G*G*T* |
| GpG.1 | T*G*A*C*T*G*T*G*G*T*G*G*T*T*A*G*A*G*A*T*G*A* |
| GpG.2 | C*T*G*T*G*G*T*G*G*T*T*A*G*A*G*A* |
| GpG.3 | C*T*C*T*G*T*G*G*T*T*A*G*A*G* |
| GpG.4 | C*T*C*T*G*T*G*G*T*T*C*C*C*C* |
| GpG.5 | G*A*G*A*G*T*G*G*T*T*A*G*A*G* |
| GpG.6 | G*A*G*A*G*T*G*G*T*T*C*C*C*C* |
| GpG.7 | C*C*G*A*G*T*G*G*T*T*A*C*G*G* |
| GpG.8 | T*G*G*C*G*T*G*G*C*C*T*G*G*C* |
| GpG.9 | A*A*A*A*G*T*G*G*T*T*C*C*C*C* |
| GpG.10 | A*A*A*A*G*T*G*G*C*C*T*T*T*T* |
| GpG.11 | A*A*AAGTGGCCTTT*T* |
| GpG.12 | A*A*A*A*G*T*G*G*T*T*A*A*A*A* |
| GpG.cc | T*G*A*C*T*G*T*G*G*T*G*G*C*C*A*G*A*G*A*T*G*A* |
| I41 | G*C*T*G*T*G*G*T*T*C*C*T* |

POLY G + RYGGY CLASS

| | |
|---|---|
| I2 | T*T*A*T*G*T*G*G*T*T*C*C*T*G*A*C*C*A*G*G*G*G* |
| | G* |
| I3 | A*T*T*A*T*G*G*G*G*T*G*T*G*G*T*T*T*T*C*C*A*C* |
| | A*C*C*C*C*G*G*G*G*G* |
| I4 | A*T*T*A*T*G*G*G*G*T*G*T*G*G*T*T*T*T*C*C*A*C* |
| | A*C*C*C*C* |
| I11 | A*T*T*A*T*GGGGTGTGGTTTTCCACACCCCG*G*G*G*G |
| I13 | T*G*A*C*T*G*T*G*G*T*G*G*T*T*A*G*A*G*A*T*G*G* |
| | G*T* |
| I14 | T*G*A*C*T*G*T*G*G*T*G*G*T*T*A*G*A*G*A*T*G*G* |
| | G*T*T*T*T*G*G*G*T* |
| I16 | T*G*T*G*G*T*T*ACAG*T*G*G*T*TGTG*G*T*T*G*G*G* |
| | G* |
| I17 | C*C*A*T*G*T*G*G*T*T*A*T*G*G*G*G* |
| I18 | C*C*A*T*G*T*G*G*T*T*A*T*G*G*G*T* |
| I21 | T*G*G*T*G*G*T*T*T*T*G*G*G*C*G*C*G*C*G*C*C*G |
| I23 | G*G*TGCAT*G*G*T*TGCAG*G*G*G*G*G* |
| I27 | C*C*T*C*A*T*G*G*T*T*G*A*G*G*G*G* |
| I28 | G*G*G*G*C*C*A*T*G*T*G*G*T*T*A*T*G*G*G*G* |
| I29 | T*G*C*T*G*C*A*C*A*T*G*G*T*T*G*A*G*G*G*G* |
| I30 | G*G*G*G*G*G*T*G*C*T*G*C*A*C*A*G*T*G*G*T*T*C* |
| | A*G*G*G*G*G*G* |
| I31 | C*C*T*C*A*T*G*G*C*C*A*A*G*G*G*G* |
| I33 | T*G*G*G*T*G*T*G*G*T*T*A*T*G*G*G*T* |
| I36 | C*C*A*C*G*T*G*G*C*C*A*T*G*G*G*T* |
| I39 | C*C*A*T*G*T*G*G*T*T*A*T*G*G*G*T* |
| I40 | T*G*G*T*G*G*T*T*G*G*G*T* |
| I18.2 | C*C*T*G*T*G*G*T*T*A*T*G*G*G*T* |
| I18.3 | T*C*C*T*G*T*G*G*T*T*A*T*G*G*G*T* |
| I18.4 | T*G*G*T*G*T*G*G*T*T*A*T*G*G*G*T* |
| I18.6 | C*C*GTGGTTGG*G*T* |
| I18.7 | C*A*G*T*G*G*C*C*T*G*G*G*T* |
| I18.8 | A*A*A*G*T*G*G*C*C*T*G*G*G*T* |
| I18.9 | C*A*G*T*G*G*C*C*T*G*G*G*T* |
| I18.10 | C*C*A*G*T*G*G*C*C*T*G*G*G*T* |
| I18.11 | C*C*A*GTCCCCTGG*G*T* |
| I18.14 | A*A*AAGTGGCCTTTGGGTC*C* |
| I18.15 | C*C*A*A*G*T*G*G*T*T*A*T*G*G*G*T* |
| I18.16 | G*C*A*T*G*T*G*G*T*T*A*T*G*G*G*T* |
| I18.19 | A*A*A*A*G*T*G*G*T*T*A*T*G*G*G*T* |

Multiple RYGGYY Motifs

| | |
|---|---|
| I8 | T*G*T*G*G*T*T*A*C*A*G*C*G*G*T*T*G*T*G*G*C*C* |
| I9 | T*G*G*T*G*G*T*G*T*G*G*C*C*A*C*A*G*T*G*G*T*T* |
| | G*T*G*G*C*C* |
| I10 | T*G*G*T*G*G*T*G*T*G*G*C*C*A*C*A*G*T*G*G*T*T* |
| I12 | T*G*T*G*G*TT*ACAGCGGTTGTG*G*T*T |
| I15 | T*G*T*G*G*T*T*ACAG*T*G*G*T*T*GTG*G*T*T* |
| I22 | T*G*G*T*G*G*T*T*T*T*G*T*G*G*T*T*T*T*G*T*G*G* |
| | T*T* |
| I26 | G*G*T*T*G*G*T*G*T*G*G*T*T*G*G*A*C*A*G*T*G*G* |
| | T*T*G*T*T*G*G*T*T*G*G*T*G*T*G*G*T*T*G*G* |
| I34 | T*G*G*T*G*G*T*G*T*G*G*C*C*A*C*A*G*T*G*G*C*C* |
| | G*T*G*G*C*C* |
| I37 | T*G*C*T*G*C*T*G*T*G*G*C*C*A*G*A*G*T*G*G*C*C* |
| | G*T*G*G*C*C* |

Multiple RYGGYY Motifs + PolyG

| | |
|---|---|
| I35 | T*G*G*T*G*G*T*G*T*G*G*C*C*A*C*A*G*T*G*G*C*C* |
| | A*G*A*G*T*G*G*C*C*T*G*G*G*T* |
| I38 | T*G*C*T*G*C*T*G*T*G*G*C*C*A*C*A*G*T*G*G*C*C* |
| | G*T*G*G*C*C*T*G*G*G*T* |
| I42 | C*C*A*GTGGCCCAGTGGCCTGG*G*T* |
| I43 | C*A*G*T*G*G*C*C*C*A*G*T*G*G*C*C*T*G*G*G*T* |

RYGGYY + G-TETRAD

| | |
|---|---|
| I24 | C*C*A*T*G*T*G*G*T*T*A*T*G*G*T*G*T*G*G*T*G*T* |
| | G*G*T*G*T*G*G* |
| I25 | T*G*G*T*G*G*T*G*T*G*G*C*C*T*G*G*T*G*T*G*G*T* |
| | G*T*G*G*T*G*T*G*G* |

Human PBMC were isolated from healthy donors at the Stanford Blood Bank. Acid citrate dextrose was used as the anticoagulant and leukocyte-rich buffy coat (approximately 30 mls). In three 50ml conicals 10mls each buffy coat was diluted 1:4 with PBS, underlayed with 8 mls of IsoPrep (1.077g/ml, pH 6.8, 9.6% w/v Sodium Metrizoate, 5.6% w/v Polysaccharide), and centrifuged without break at 400 g for 30 min at room temperature. The interphase cells (lymphocytes and monocytes) were transferred to a new 50 ml conical tube, filled with PBS, mixed and centrifuged at 200 g for 10 min at room temperature. The supernatant was removed and the wash step repeated. The final cell pellet was resuspended in 5 mls bead buffer (PBS pH7.2, 0.5% BSA, 2 mM EDTA), the cells counted using ViCell (Beckman-Coulter), and cultured in RPMI-1640 with 10% FBS.

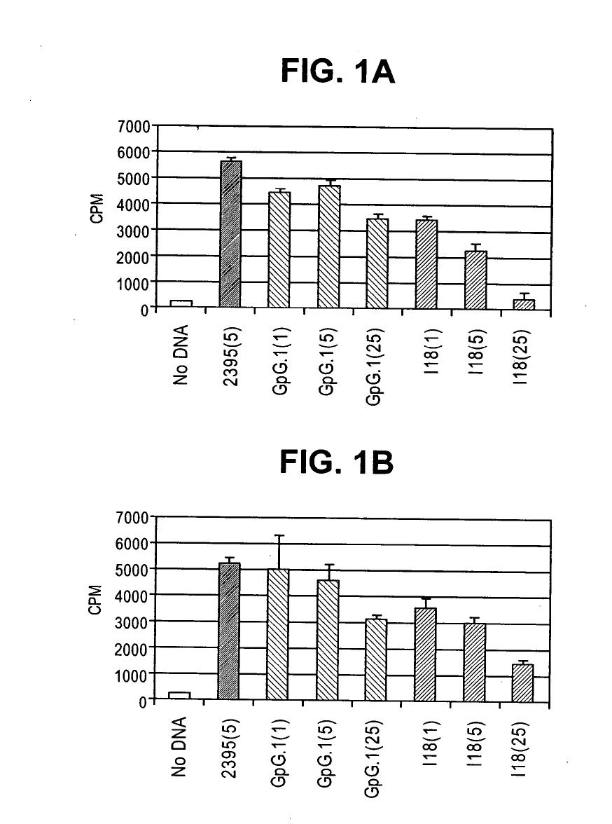

To determine if IMS could inhibit CpG ISS ODN stimulation of cell proliferation, PBMCs were incubated with single or increasing doses of IMS in the presence of 5 μg/ml ISS ODN for 4 days. Cell proliferation was assayed by measuring [³H] thymidine incorporation during the last 24 hrs of incubation. The effectiveness of the inhibition varied significantly between IMS ODN (˜15-70% inhibition at the 5 μg/ml g/ml dose; FIG. 1a, b) and increasing the dose of the IMS tested from 1 to 25 μg/ml increased the inhibition of the proliferative response to ISS

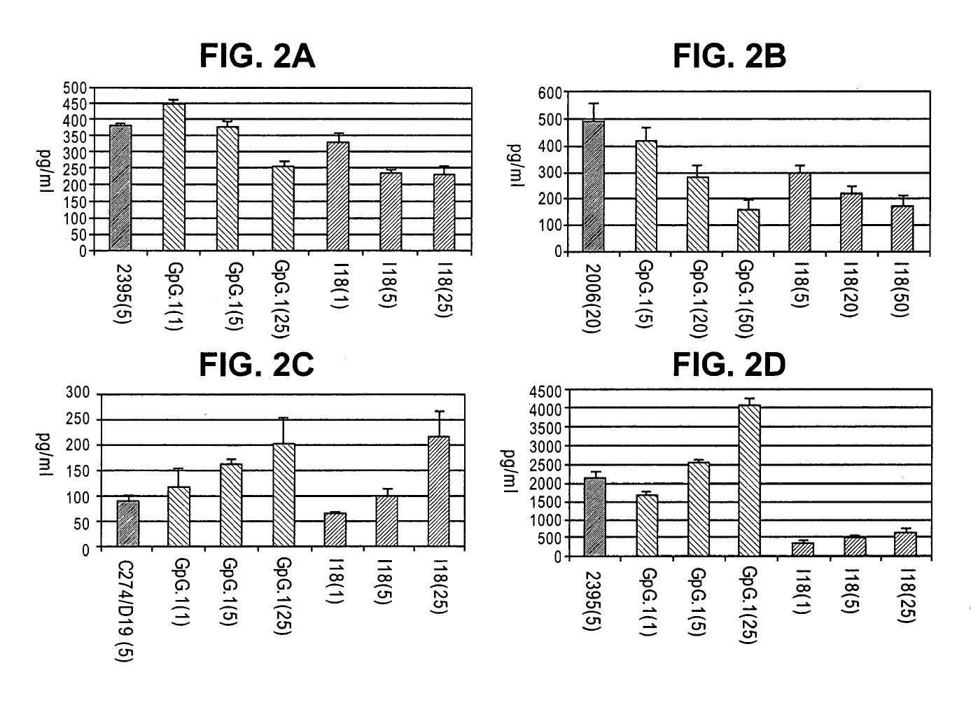

To profile the effect of the IMS on CpG-ODN stimulated cytokine production, hPBMCs were incubated for 48 hours with the indicated concentrations of IMS and stimulatory CpG-ODN and cytokine levels in the culture medium were analyzed by ELISA. As shown in FIG. 2, the IMS suppressed CpG stimulated IL-10 and IL-12 expression in a dose dependent manner. In contrast IMS generally enhanced CpG induced IFN-gamma expression particularly at the 25 μpg/ml dose, whereas differential IMS affects on IFN-alpha expression were observed. While the IMS I18 typically suppressed CpG induction of IFN-alpha, IMS like GpG.1 enhanced expression (FIG. 2c, d).

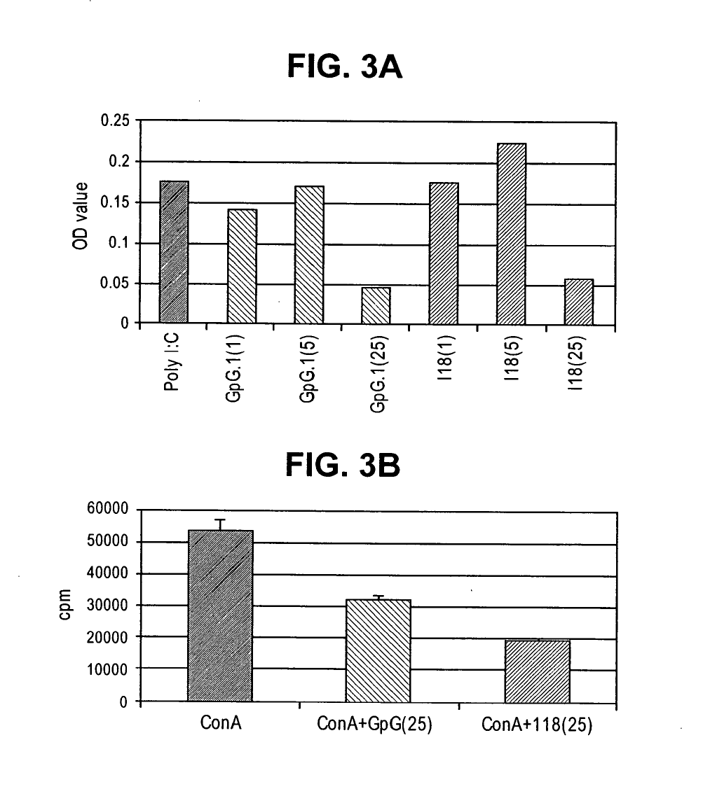

In addition to inhibiting CpG stimulated immune responses, the I18 and GpG.1 oligos also inhibit ConA dependent cell proliferation and Poly I:C stimulated IFN-alpha expression in PBMC cultures (FIG. 3). ConA acts directly on T cells, and Poly I:C has been shown to induce IFN-alpha expression in a subset of human monocytes. Published data suggests that these cells do not express functional TLR9 receptors (Hornung et al., J. Immunol. 168:4531) suggesting that the IMS of the present invention affect immune responses in a TLR9 independent manner consistent with published results for mouse immune cells (Shirota et al., J. Immunol. 173:5002).

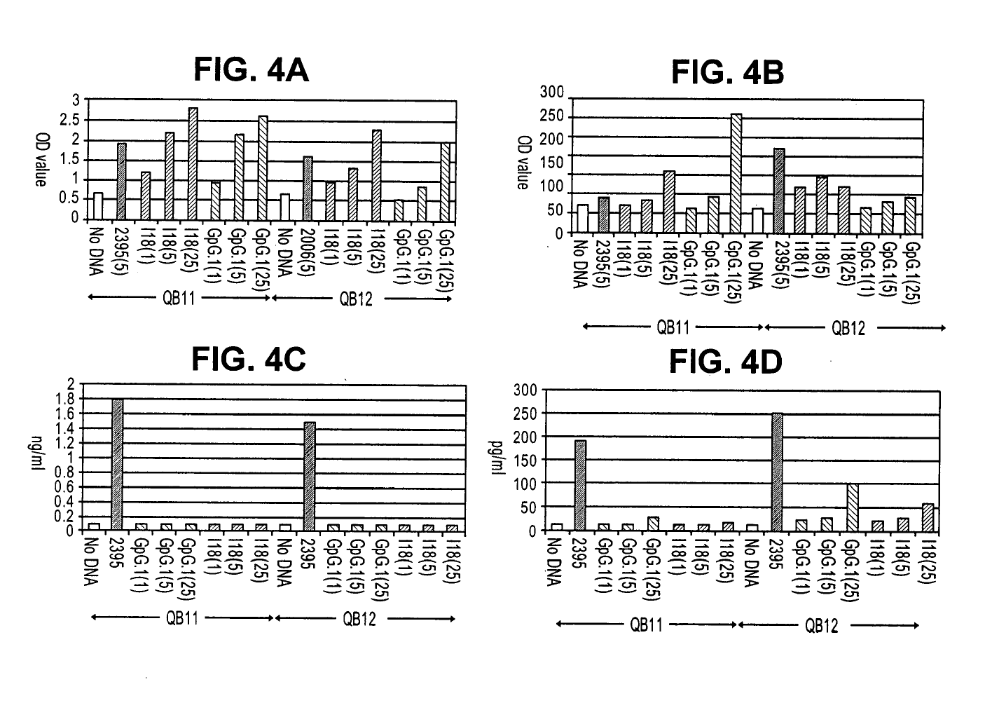

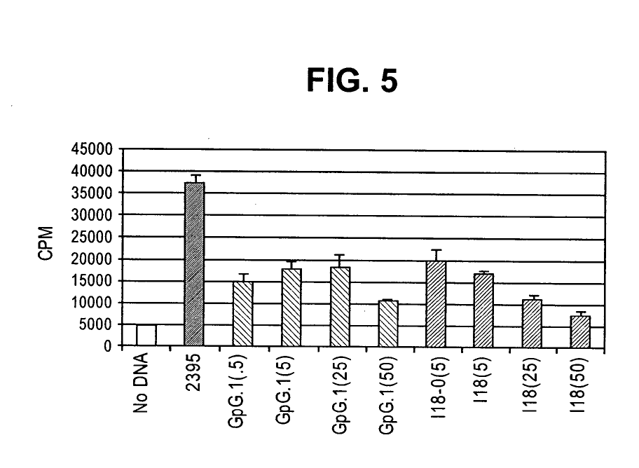

Published studies have demonstrated that phosphorothioated non-CpG ODN can have immune stimulatory properties similar to those of CpG ODN. Specifically, these oligos can cause B cell activation resulting in B cell proliferation and secretion of IL-6 and IL-10 (Vollmer et al., Immunol. 113:212; Liang et al., J. Clin. Invest. 98:1119; Vollmer et al., 2002, Antisense Nucleic Acid Drug Dev. 12:165-75). To determine if the IMS of the present invention stimulate these effects in PBMC cultures, we incubated cells with increasing concentrations of IMS in the absence of CpG-ODN. Proliferation (FIG. 5) and secretion of IL-6,11-10, and IFN-gamma production were all stimulated by >25 μg/ml of IMS (FIG. 4a, b, d). In contrast, induction of IFN-alpha was not observed at any of the oligo concentrations used (FIG. 4c).

### Example 2

### IMS-ODN Inhibit CpG-ODN Induced Cytokine and Chemokine Production In vivo

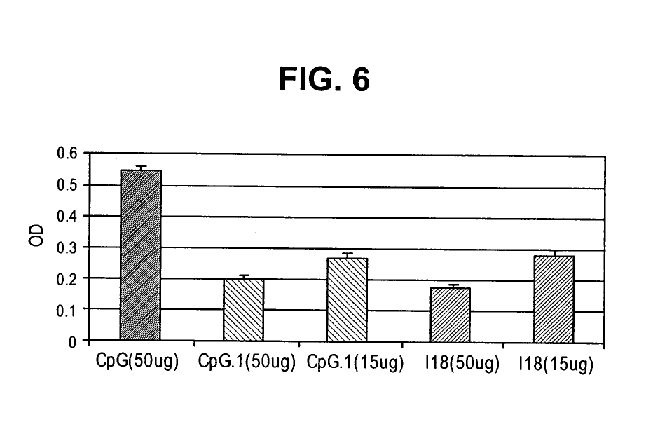

To determine if IMS-ODN can suppress CpG-ODN effects in vivo, mice were injected with a mixture of CpG and IMS oligos. To examine the in vivo kinetics of IMS action 50 μg of I18 was injected IP into 4 groups of mice (D0-D3;n=3). A stimulatory CpG-ODN (mCpG) was injected into Group 1(D0) simultaneously with I18; Group 2 (D1)-24 hrs after I18; Group 3 (D2)-48 hrs after I18; and Group 4 (D3)-72 hrs after I18. Twenty-four hours post injection, serum was collected and analyzed by ELISA for expression of the pro-inflammatory proteins IL-12 and MCP-1. FIG. 6 demonstrates that significant inhibition of IL-12 can be observed at both 1:1 and 1:3 mass ratios of IMS:CpG ODN. A significant inhibition of MCP-1 levels was also observed (data not shown).

### Example 3

### IMS Biological Effect Persists for Several Days In vivo

In vitro studies have shown that the inhibitory effects of some IMS on CpG-ODN can persist for 16 hrs (Stun et al., Eur. J. Immunol. 32:1212). In order to examine the persistence of the IMS effects in vivo, mice were injected with IMS at Day 0 and then injected with a stimulatory CpG ODN at Day 1, 2 or 3. Serum was collected 24 hrs after CpG injection and IL-12 was measured. FIG. 6 demonstrates that IMS injected at Day 0 still inhibits the effects of CpG injected 3 days later.

### Example 4

### IMS Delay Disease Onset in a Mouse Model of SLE

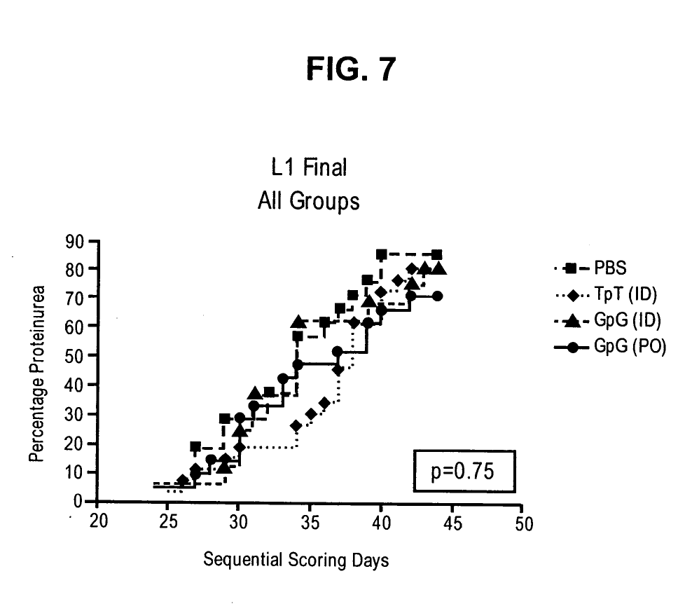

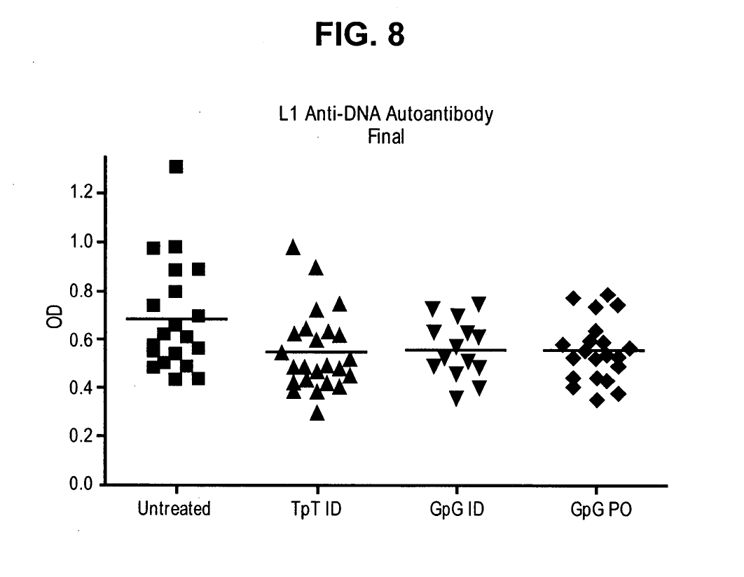

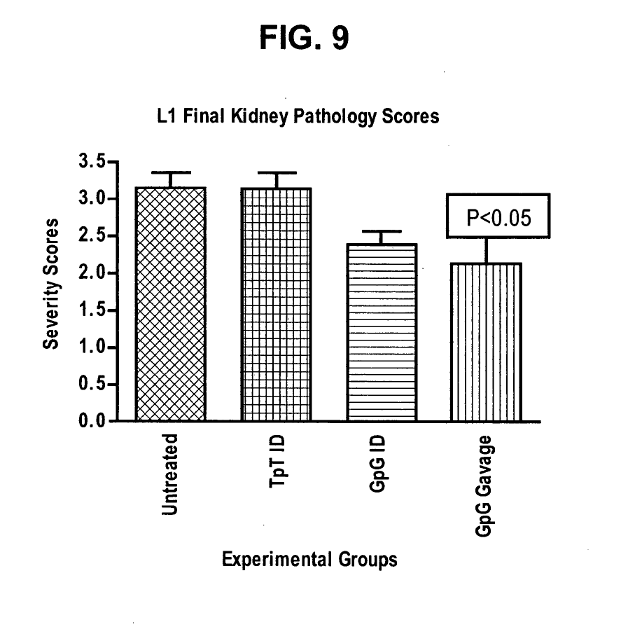

IMS oligos were tested for their ability to affect disease onset in an animal model of lupus. NZB/W F1 female mice spontaneously develop proteinurea, kidney pathology and antibodies to DNA similar to individuals with systemic lupus erythematous (SLE). TpT and GpG IMS oligos were administered to NZB/W F1 female mice at 50 μg weekly by intradermal delivery (ID). Alternatively, GpG IMS oligos were administration by oral gavage (PO; 50 μg, QW). Control animals received weekly injections of the vehicle, PBS. Although no significant delay in proteinurea onset was observed in any of the experimental groups (FIG. 7) and autoantibody responses to DNA were not decreased by a statistically significant amount (FIG. 8), analysis of the kidneys revealed a significant effect of the GpG oligo in decreasing inflammation when the oligo was administered by oral gavage (FIG. 9). The GpG delivered by ID administration also lowered the scores, but this did not reach statistical significance.

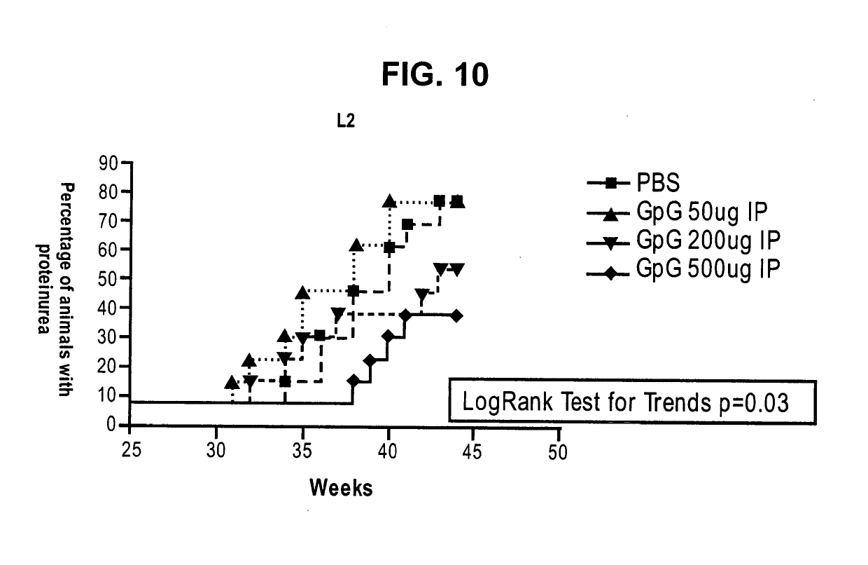

Given the effect of 50 μg GpG IMS oligos on kidney pathology in this mouse model of SLE, we performed experiments to examine a dose response. 50, 200 and 500 μg of GpG IMS oligo were administered to NZB/W F1 female mice weekly by IP injection. A dose dependent delay in proteinurea onset and decrease in autoantibody response to DNA were observed, with a highly significant delay in proteinurea onset and lowest median DNA autoantibody titer in mice injected with 500 μg GpG IMS oligo (FIGS. 10 & 11). Kidney pathology will be performed on these animals.

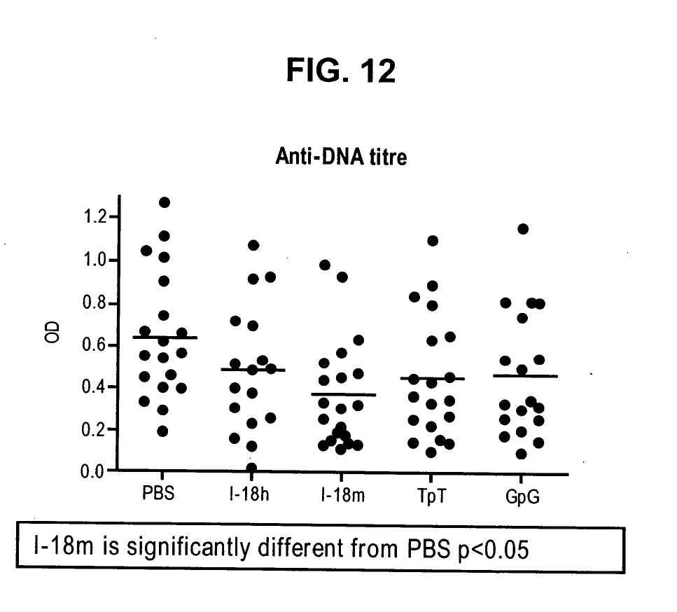

In vitro experiments described above demonstrated that a third oligo, I-18, may be qualitatively different from the TpT and GpG oligos. To compare the effect of these different oligos in lupus 50 μg of TpT, GpG and I-18 (both human and mouse, I-18h and I-18m, respectively) oligos were administered to NZB/W F1 female mice daily by IP injection. Animals were sacrificed at week 34, a time at which approximately 30% of the control group exhibited proteinurea. Autoantibody analysis revealed a significant decrease in anti-DNA response in the I-18m treated group compared to vehicle treated control groups (FIG. 12). Kidney pathology will be performed on these animals.

### Example 5

### IIS Oligos Decrease the Severity of Inflammation in Mice with Experimentally Induced Uveitis

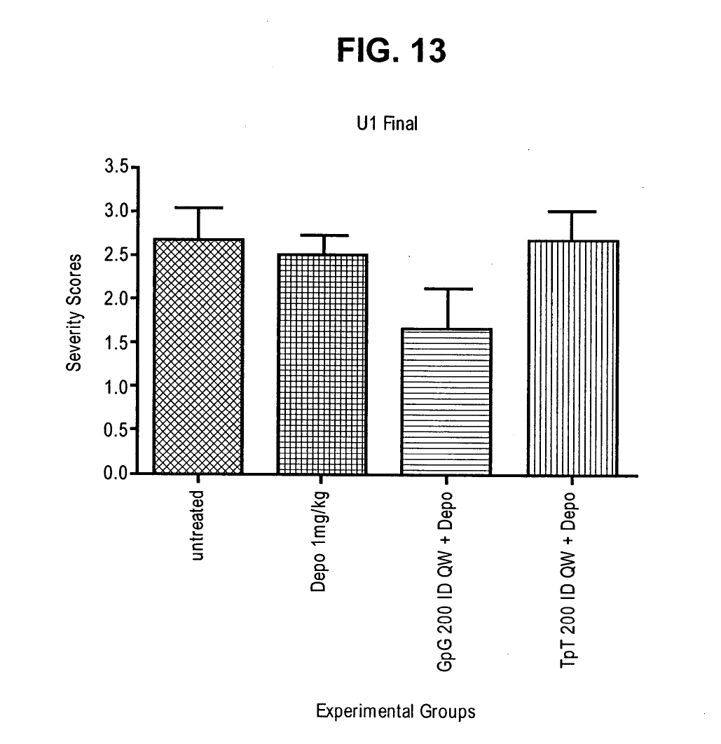

To determine if the efficacy observed in the lupus animal model generalized to other autoimmune diseases, the effect of IMS oligos on uvietis, an autoimmune disease on the eye, was examined. Experimentally induced autoimmune uveitis (EAU) is a mouse model of uvietis that has many common features with the human disease (Animal Models for Autoimmune and Inflammatory Disease, Current Protocols in Immunology, 2003 Chapter 15.6). EAU was induced in B10.RIII mice by immunization with a peptide fragment of the human intraretinal binding protein, hIRBP₁₆₁₋₁₈₀, emulsified in CFA. 200 μg of each IMS oligo was then administered weekly by ID injection in combination with a low dose of the steroid depromedrol (1 mg/kg), which is the standard of care for human uveitis. Extent of EAU was scored by orbit pathology at day 21. A trend towards lowering of disease severity with the administration of GpG IMS oligo and low dose steroid was observed (FIG. 13) whereas TpT showed no synergistic affect with steroid.

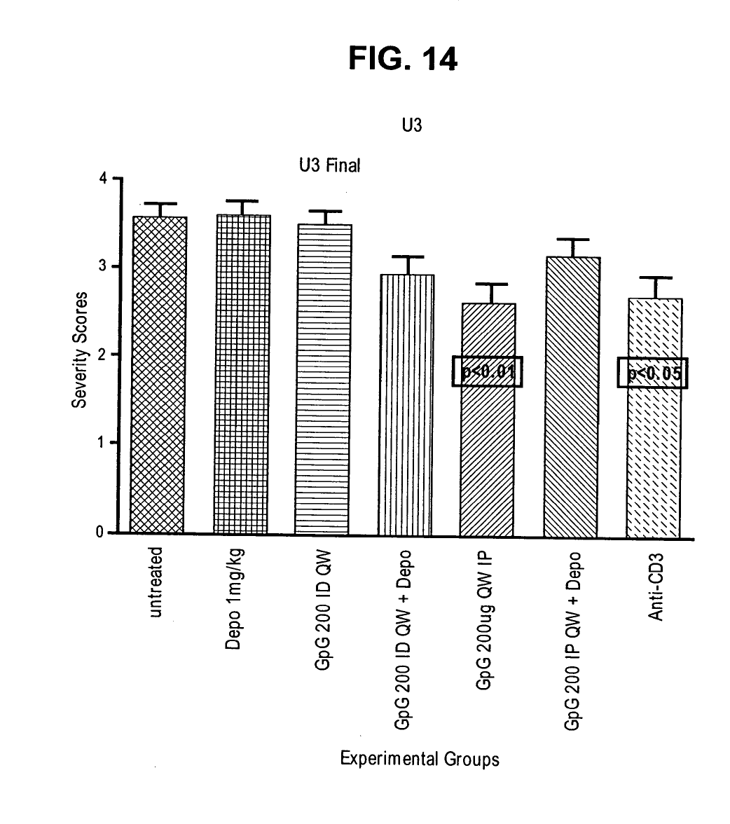

To extended these observations, IMS oligo in the absence of steroid treatment and intradermal versus intraperitoneal dosing were examined. EAU was induced in B10.RIII mice by immunization with hIRBP₁₆₁₋₁₈₀ peptide emulsified in CFA. 200 μg of each IMS oligo was then administered weekly by IP or ID injection alone or in combination with a low dose of the steroid depromedrol (1 mg/kg). As a positive control, anti-CD3 antibodies were administered daily for 5 days beginning at day 0 at 5 μg per animal by IV administration. Whereas weekly intradermal or intraperitoneal delivery of GpG IMS oligo plus steroid group resulted in lower severity scores than steroid only, neither were statistically significant (FIG. 14). In contrast, administration of GpG oligo alone by IP was more efficacious than when used in combination with steroid treatment and resulted in a statistically significant improvement in disease severity compared to untreated controls (p<0.01) (FIG. 14). This effect was comparable to a positive control group treated with anti-CD3 (p<0.05).

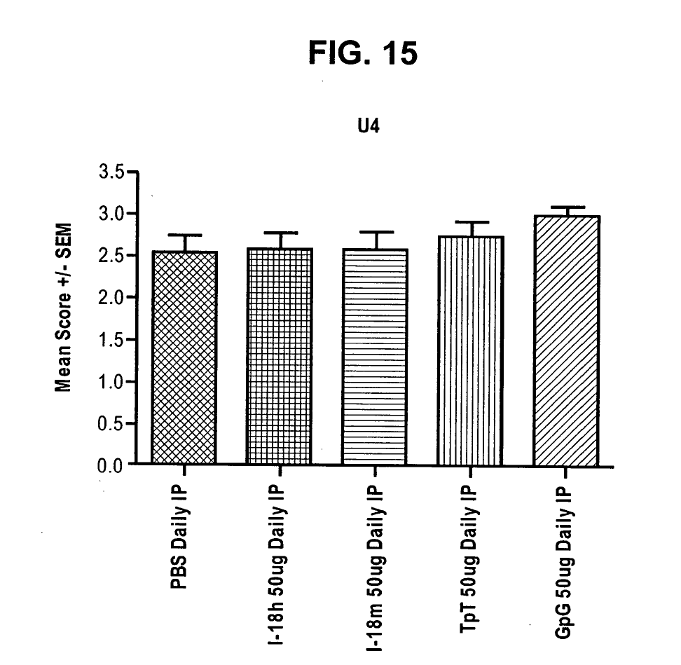

To further analyze the effect of the IMS oligos on EAU and determine the lowest effective dose, we compared IP administration of 50 μg GpG, TpT, I18h and I18m oligos. In contrast to the weekly IP dosing with 200 μg of GpG (FIG. 14), daily 50 μg dosing with GpG or any of the other IMS oligos provided no significant improvement in disease severity (FIG. 15).

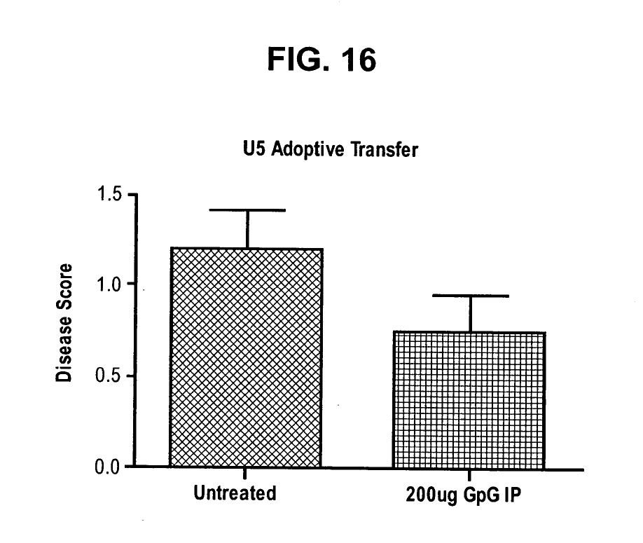

As EAU is induced with CFA, one possible mechanism of action by which GpG oligos lower disease severity is by competing with CpGs in the mycobacterium component of CFA. To examine the effect of GpG IMS oligos on disease course in the absence of CFA, adoptive transfer experiments were performed. Uveitogenic cells induced in animals treated with hIRBP₁₆₁₋₁₈₀ peptide/CFA were harvested and grown in vitro for 3 days with hIRBP₁₆₁₋₁₈₀ peptide. On day 4, the cells were adoptively transferred to naïve recipients, half of which received weekly IP injections of 200 μg GpG oligos and half received PBS vehicle as a control. Animals treated with GpG oligos showed less severe inflammation than the vehicle treated group (FIG. 16), suggesting that the GpGs may have effects on disease that are not related to a CpG blocking effect.

### Example 6

### HS Oligos Delay Onset and Lower Severity in an Animal Model of Arthritis

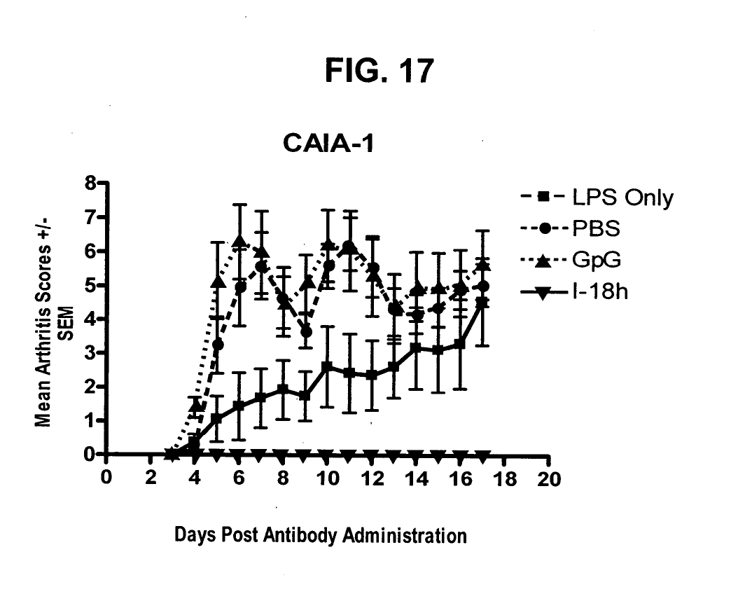

The IMS oligos of the present invention were next tested in an arthritis model of autoimmune disease where, instead of T-cells as in EAU, antibodies were driving the inflammation. Collagen antibody-induced arthritis (CIA) was induced in Balb/c mice by a single IV injection of 200 μg of four monoclonal anti-collagen arthritogenic antibodies on day 0 (Terato, K. et al. 1992), and two days later the disease was synchronized by injection of LPS. Thus no mycobacterial DNA or other exogenous sources of CpGs were utilized to induce disease. GpG and I18h IMS oligos were then were administered at 50 μg by IP on day 4 thru day 10. Animals were observed daily using the following scoring system: 0=Normal; 1=Erythema with mild swelling confined to the mid-foot (tarsal) or ankle joint; 2=Erythema and mild swelling extending from the ankle to the mid-foot; 3=Erythema and moderate swelling extending from the ankle to the metatarsal joints; and 4=Erythema and severe swelling encompass the ankle, foot and digits. Each paw could be assigned a maximum score of 4 and each mouse a maximum score of 16. The mean arthritis score was determined by averaging the arthritis scores for each paw from animals in each experimental group. Whereas treatment with 50 μg GpG oligo provided no decrease in disease severity or disease incidence, a significant decrease in arthritis severity and delay in onset was observed in animals treated with I8h oligos (FIGS. 17 & 18).

### Example 7

### IIS Oligos Inhibit Weight Loss in Mouse Models of Colitis

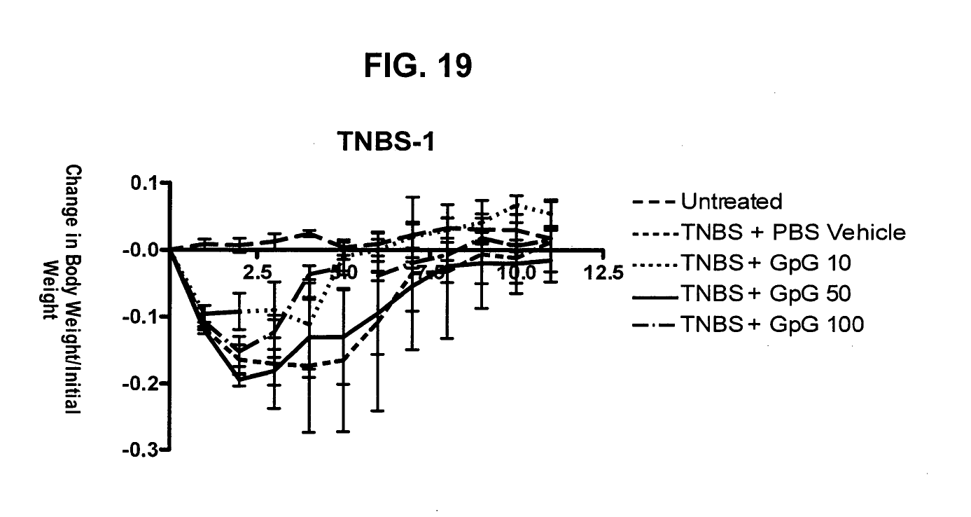

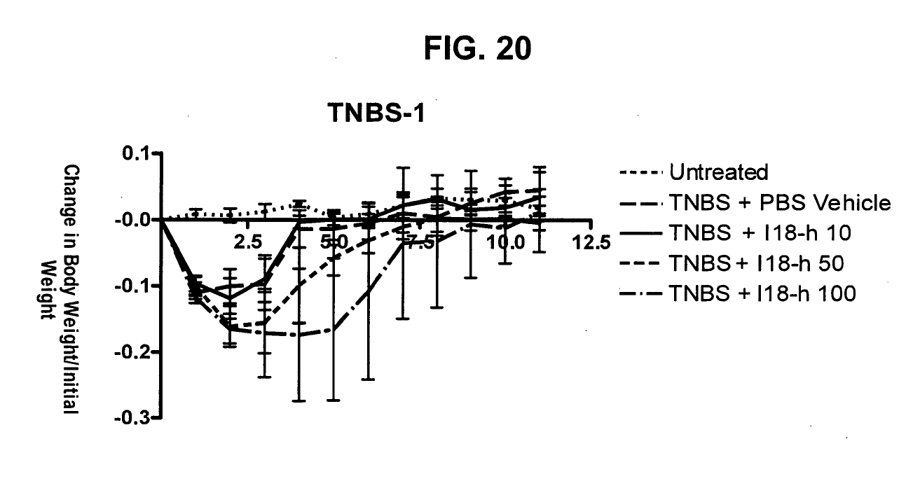

Published studies have suggested that CpG oligos minimize weight loss in animal models of colitis (Rachmilewitz, D. et al. 2002). In some studies, however, the timing of the dosing was critical with pre-treatment providing a significant protective effect, but treatment after disease onset exacerbating disease (Obermeier, F. et al., 2003; Obermeier, F., 2002). To determine if IMS oligos of the present invention could similarly affect colitis, an IL-12 mediated animal model of inflammatory bowel disease, the TNBS induced colitis model, was used (Animal Models of Autoimmune Disease, Current Protocols in Immunology, Chapter 15.19, 2003). C3H mice were treated rectally with a sub-colitogenic dose of TNBS (0.5%) on day −5. On the same day IP treatment with GpG, I18h or I18m oligos was commenced and continued for 5 days. Disease was then induced by a second TNBS administration (3.5% rectally) after which oligo treatment was stopped. Animals were weighed daily and the change in body weight divided by the original body weight (day 0) was used to determine the mean weight loss for each treatment group. All animals treated with oligos showed decreased weight loss when compared to the vehicle control group (FIGS. 19, 20 & 21).

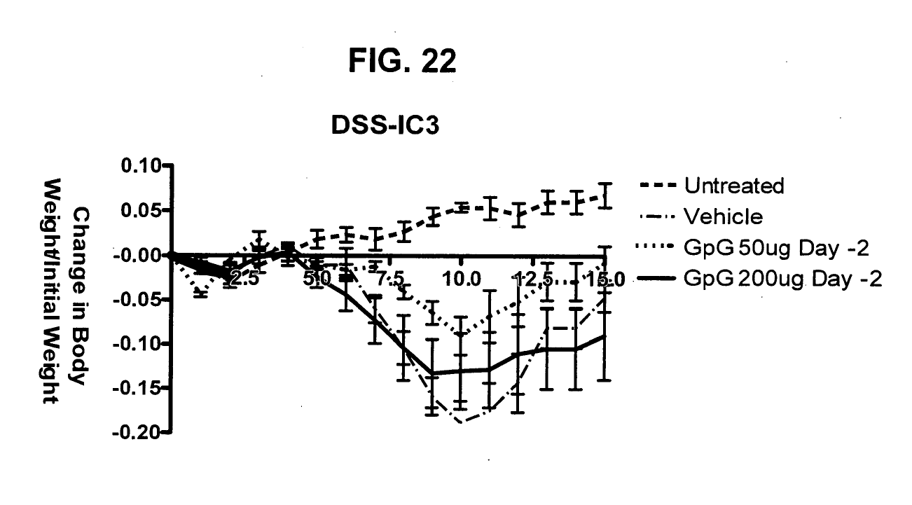

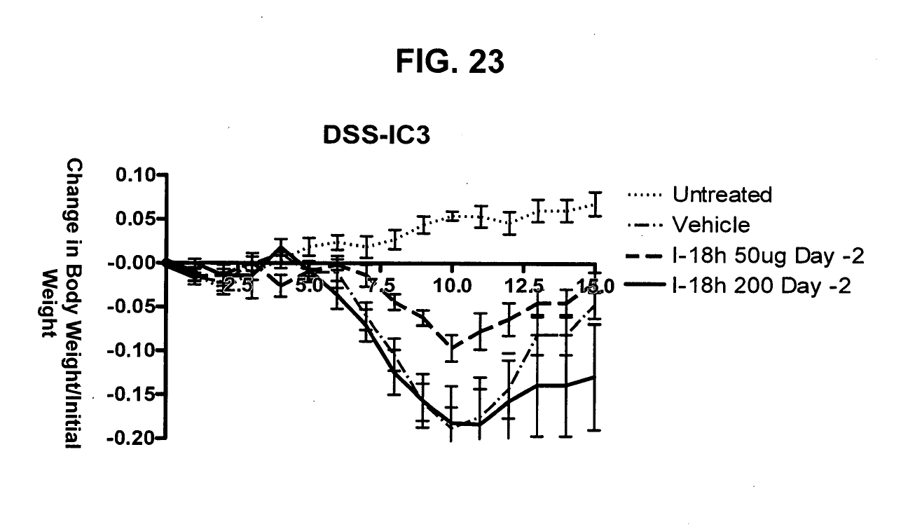

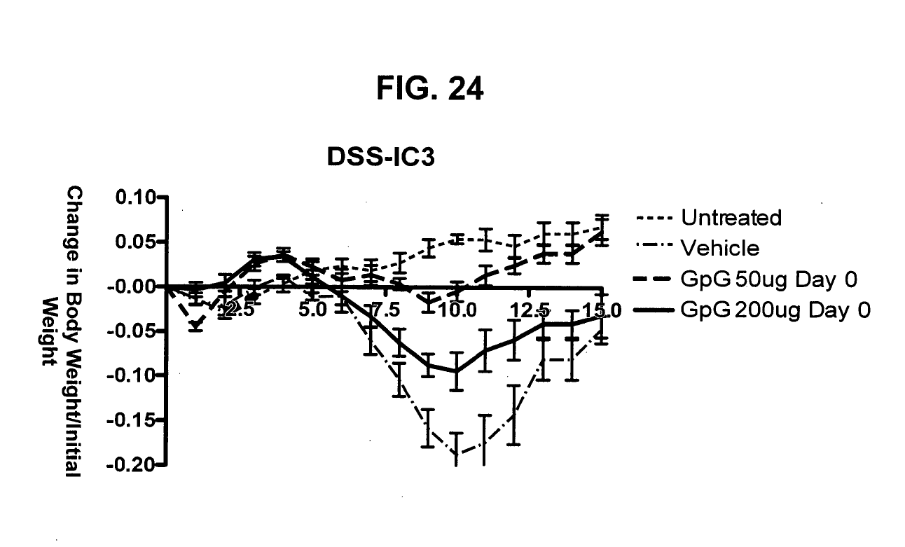

A second model of inflammatory bowel disease was also examined. Oral administration of dextran sodium sulfate (DSS) induces acute colitis that, unlike the TNBS, is exclusively mediated by the innate immune system. Female C3H mice were pretreated beginning at day −2 with a 50 or 200 μg of GpG, I-18h or I-18m oligos daily by intraperitoneal injections and then fed 3.5% DSS in drinking water for seven days (day 0-7). Alternatively, oligo treatment started on day of disease induction. Animals were weighed daily and the change in body weight divided by the original body weight (weight at day 0) was determined. In both prevention and treatment experiments, IMS oligos provided significant protection from weight loss when compared to the vehicle treated control group (FIGS. 22, 23, 24 & 25). In each case, the treatment that was started on day 0 provided the maximum protection.

### Example 8

### I18 Mutagenesis

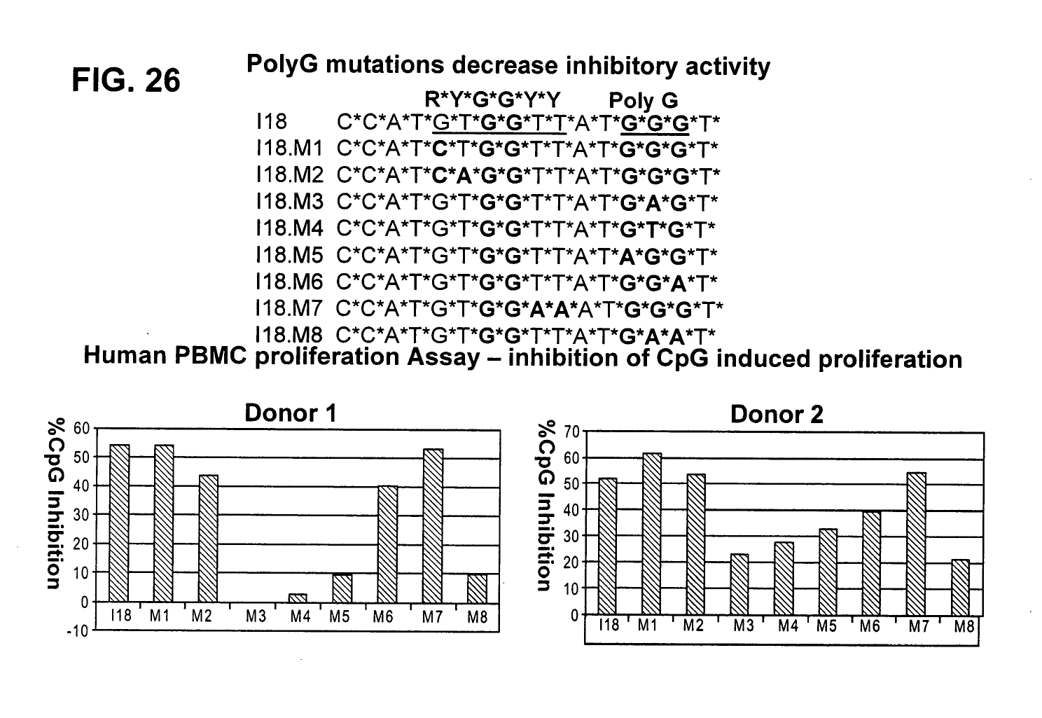

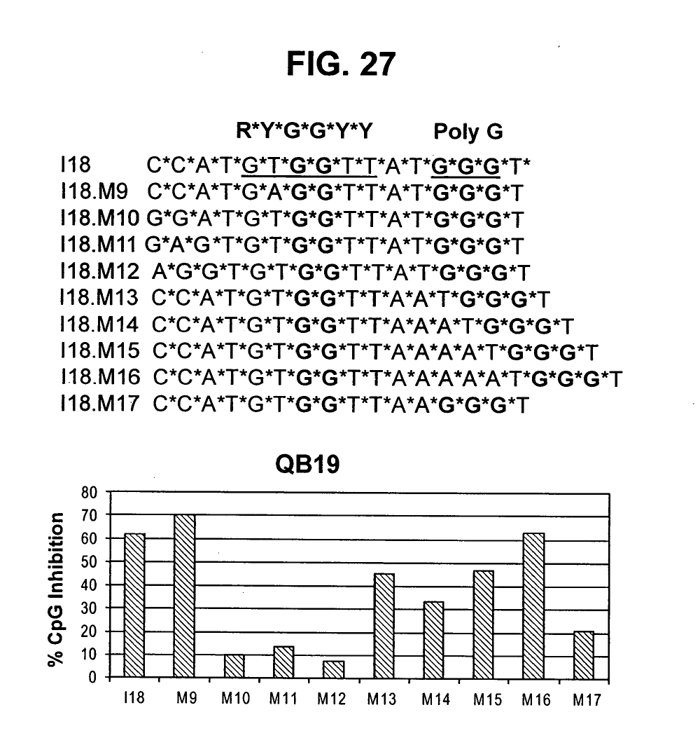

To further evaluate the structural motifs responsible for immune modulation by I18, the effect of I18 mutagenesis on CpG mediated proliferation of human peripheral blood mononuclear cells (PBMC) was determined as described above. Mutations within the polyG region (I18.M3-6 & 8; FIGS. 26) and 5′ to the hexameric sequence (I18.M10-12; FIG. 27) significantly reduced the ability of oligonucleotides containing the hexameric sequence 5′-GTGGTT-3′ to inhibit PBMC proliferation. Furthermore, addition of nucleotides between the hexameric sequence and the polyG modestly reduced PBMC proliferation (I18.M13-16; FIG. 27).

### Example 9

### I18 and Signaling through Toll-like Receptors

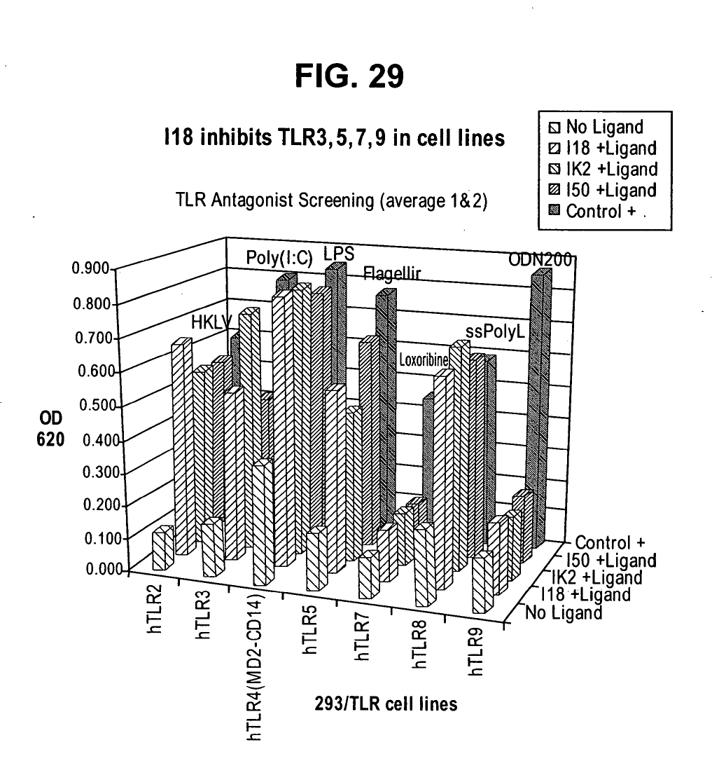

To determine the mechanism by which I18 modulates immune responses, the effect of I18 on Toll-like receptor (TLR) activation was assessed. TLR signaling was examined by NF-κB activation in cultured HEK293 cells expressing TLR2, 3, 4, 5, 7, 8 and 9. To screen for TLR agonists each immune modulatory oligonucleotide including I18 was tested in duplicate at the highest concentration (25 μg/ml), and TLR activation was compared to control ligands (listed below) for the corresponding TLR. Similarly TLR antagonists were identified by comparing mixtures of immune modulatory oligonucleotides and control ligand versus the activity of the control ligand alone. I18 inhibited activation of TLR3, 5, 7 and 9 by their corresponding ligands. See, FIG. 29. The control ligands used include: TLR2: HKLM (heat-killed Listeria monocytogenes) at 10⁸ cells/ml; TLR3: Poly(I:C) at 100 ng/ml; TLR4: E. coli K12 LPS at 10 ng/ml; TLR5: S. typhimurium flagellin at 10 ng/ml; TLR7: Loxoribine at 1 mM; TLR8: ssPolyU/LyoVec at 50 μg/ml; TLR9: CpG ODN 2006 at 1 μg/ml.

### Example 10

### I18 Inhibits TLR7 and TLR3 Ligand Induced Production of IFN-Alpha

Plasmacytoid dendritic cells (pDCs) are a major endogenous source of IFN-alpha and a source of elevated IFN-alpha levels in patients with systemic lupus erythematous (SLE). To determine if the IMS I18 can affect IFN-alpha production by pDCs in response to TLR7 agonists, pDCs were isolated and incubated with TLR7 agonist with or without I18.

Human pDCs were separated from PBMC isolated by density gradient centrifugation from two different donors using IsoPrep. The cell suspension was centrifuged at 300 g for 10 minutes and the supernatant was discarded. The cell pellet was resuspended in 400 uL of bead buffer (PBS pH 7.2, 0.5% BSA and 2 mM EDTA) per 10⁸ cells. 100 uL of the Non-PDC Biotin-Antibody Cocktail was added per 10⁸ cells, mixed and incubated for 10 min at 4-8° C. Cells were washed with 5-10 ml of bead buffer per 10⁸ cells, centrifuged at 300 g 10 minutes, and the supernatant was removed. The cell pellet was resuspended in bead buffer (400 ul/10⁸ total cells) and Anti-Biotin Microbeads (100 ul/10⁸ total cells) mixed well and incubated for 15 min at 4-8° C. The cells were then washed by adding 5-10 mL of bead buffer per 10⁸ cells, centrifuged at 300 g for 10 minutes and the supernatant was removed. The cells were resuspended in a final volume of 500 uL/10⁸ cells and added to a LS Column that was previously washed by rinsing with 3 mL of bead buffer and positioned in a MACS magnetic column holder. The column was washed with 3×3 mL of bead buffer and the total effluent containing the unlabeled enriched plasmacytoid dendritic cell fraction was collected.

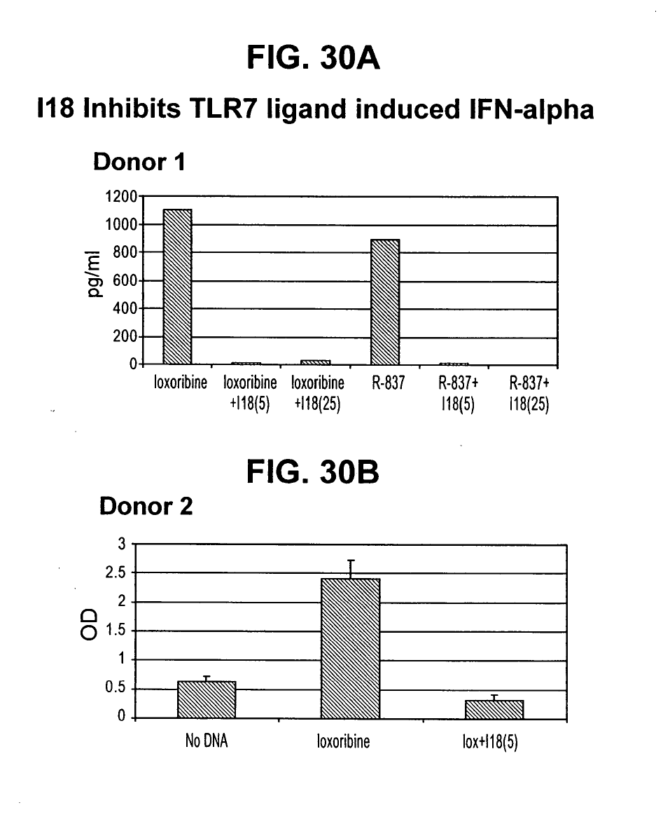

Isolated pDCs from Donor 1 were incubated with TLR7 agonists loxoribine (Invivogen; Cat #tlrl-lox) and imiquimod (R-837; Invivogen; Cat #tlrl-imq) alone or with either 5 μg/mL or 25 μg/mL I18, and IFN-alpha production was measured by ELISA (PBL Biomedicals; Cat #41105-2) according to the manufacturer's protocol. I18 at either concentration completely eliminate IFN-alpha production by pDCs (FIG. 30A). Isolated pDCs from Donor 2 were incubated without oligonucleotides, with TLR7 agonist loxoribine and loxoribine plus 5 μg/mL I18. Again, I18 completely blocked IFN-alpha production by TLR7 (FIG. 30B).

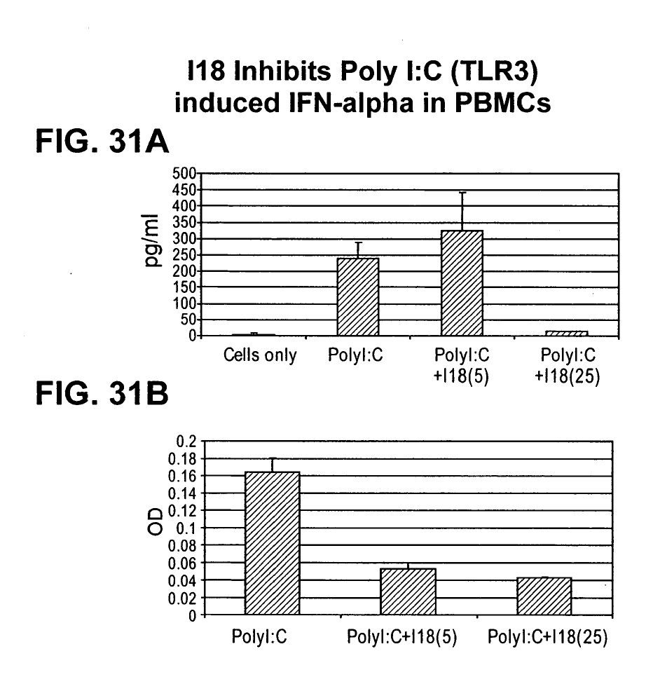

Similarly, incubation of PBMC with TLR3 agonist PolyI:C results in IFN-alpha production that is blocked in two different donors by 25 μg/mL I18 (FIG. 31).

### Example 11

### I18 Suppresses CpG Induced IFN-alpha Production by pDCs

CpG sequences present in endogenous nucleic acid immune complexes in SLE patient serum may mediate production of IFN-alpha by plasmacytoid dendritic cells (pDCs). To determine if the IMS I18 can affect IFN-alpha production by pDCs in response to CpG sequences, pDCs were isolated and incubated with CpG immune stimulatory oligonucleotides with or without I18.

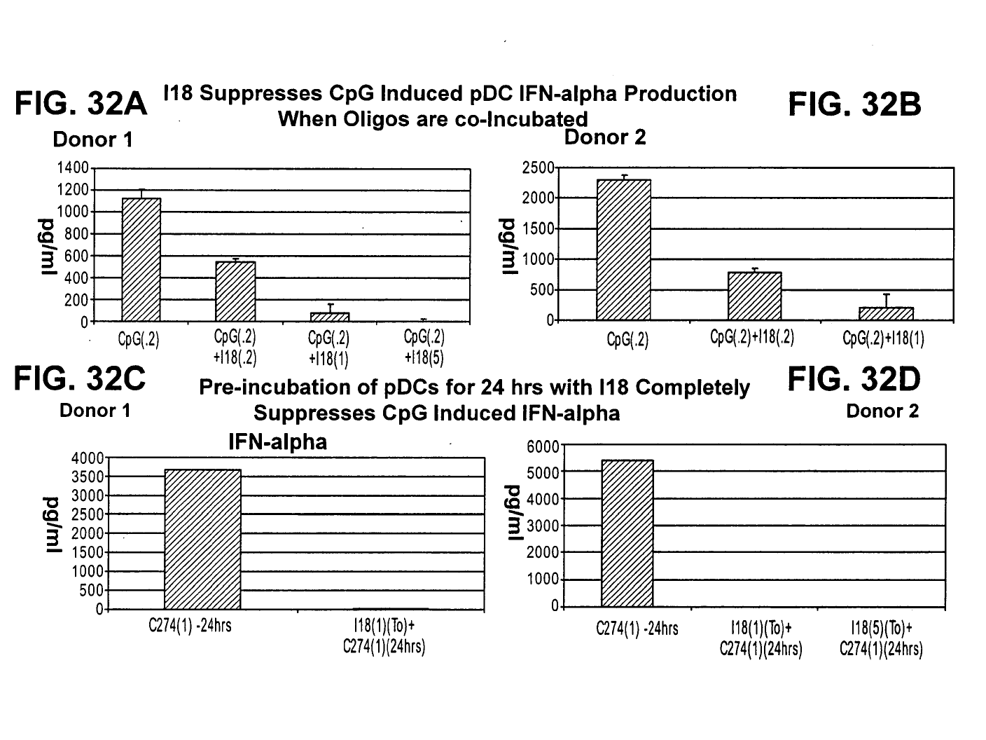

pDCs isolated as described above were incubated with CpG alone or with increasing amounts of I18. IFN-alpha production was measured by ELISA as described above. I18 significantly reduced IFN-alpha production when presented with CpG oligonucleotides at equal molar ratios and virtually eliminated production at higher ratios in pDCs from two different donors (FIG. 32A, B). Pre-incubation of pDCs with I18 for 24 hours before introduction of CpG oligonucleotides completely eliminated IFN-alpha production from both donors (FIG. 32C, D).

### Example 12

### I18 Inhibits SLE-Immune Complex Induction of IFN-Alpha in pDCs

Serum from SLE patients contains anti-dsDNA antibodies and immune complexes that contribute to the overproduction of IFN-alpha by pDC in these patients via TLR9 and FcyRIIa. To determine if I18 affects IFN-alpha production, isolated pDCs were incubated with SLE serum or SLE-ICs from four different patients and inhibition by I18 was examined.

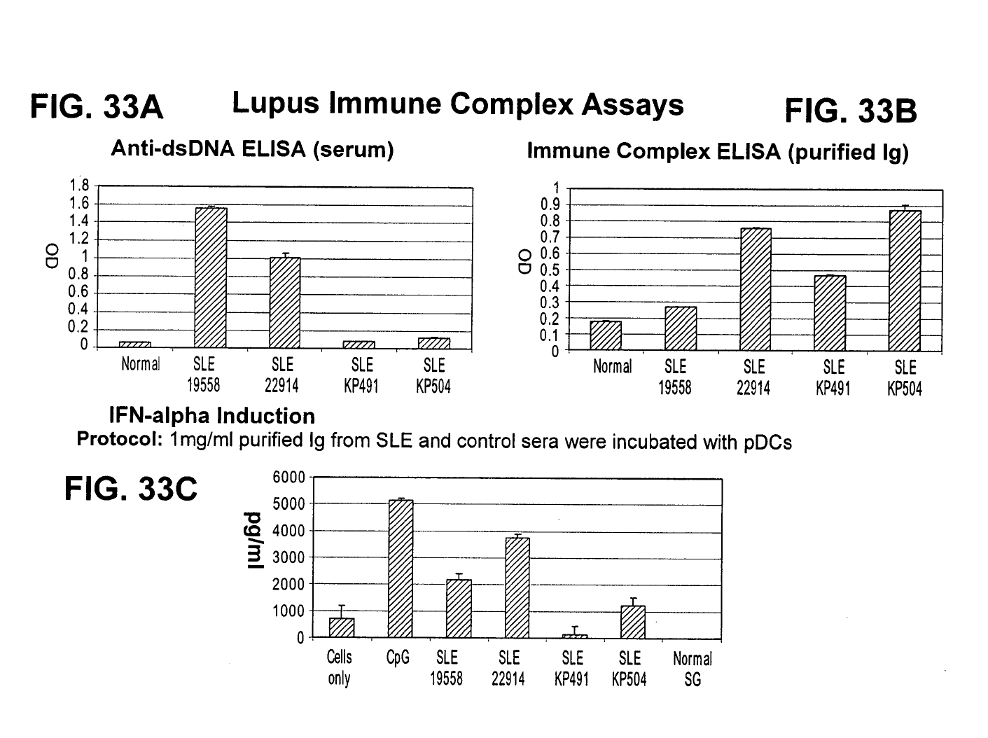

Serum isolated from SLE patients was first assessed for the presence of anti-dsDNA antibodies and immune complexes by ELISA compared to a normal control. Patients 19558 and 22914 had high levels of anti-DNA antibodies whereas patients KP491 and KP504 were near normal (FIG. 33A). Immune complexes were isolated from human sera by Protein A Agarose Fast Flow beads (2ml; Sigma P3476) in a 5 cm chromatography column (Pharmacia). The column was washed with 10 ml PBS containing 0.02% sodium azide. Human serum (1-2 mL) was diluted 1:3 in PBS and filtered through a 0.2 um syringe filter. The diluted serum was applied to a column and the column was washed with 10-15 mL of PBS, eluted with 10 mL 0.1M citric acid pH2.6 and collected into a 50 mL conical containing 2 mL 1M Tris buffer pH 7.5. The eluant was dialyzed against PBS over night, sterile filtered, and the OD280 was measured to determine protein concentration using 1.5 as the extinction coefficient. All SLE patients had higher levels of immune complexes than the normal control (FIG. 33B). Furthermore, incubation of 1 μg/mL purified Ig from SLE patients with isolated pDCs induced production of IFN-alpha only in patients with anti-dsDNA antibodies (FIG. 33C).

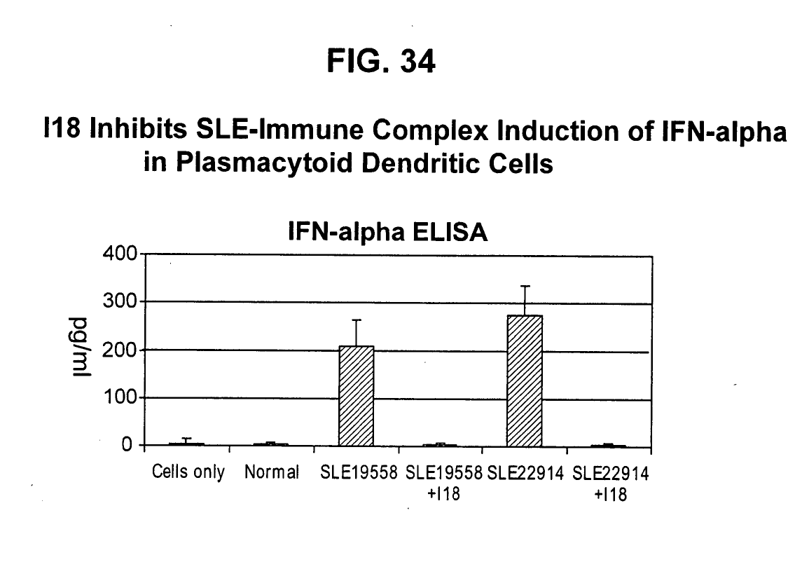

Next the ability of I18 to inhibit production of IFN-alpha by pDCs in response to immune complexes from SLE patients whose serum contains anti-dsDNA antibodies was examined. Purified Ig from SLE patients and a normal control were incubated for 24 hours with isolated pDCs in the presence or absence of I18. Isolated pDCs or pDCs incubated with immune complexes from a normal control produced little IFN-alpha (FIG. 34). In contrast, pDCs incubated with immune complexes from SLE patients produced significant amounts of IFN-alpha, and the production of IFN-alpha is inhibited by I18.

### Example 13

### I18 Inhibits CpG Activation of Normal Peripheral B Cells (CD19+)

To determine the effect of I18 on B cells activated by immune stimulatory CpG sequences, CD19+ peripheral B cells were isolated from human peripheral blood and both cytokine production and cell proliferation were examined in the presence or absence of the immune modulatory oligonucleotide I18.

CD19+ peripheral B cells were isolated from human blood PBMCs using 20 μL of CD19 MicroBeads added to 10⁷ total cells and incubated for 15 minutes at 4° C. Cells were washed with 2 mLs/10⁷ cells, centrifuged at 300×g for 10 minutes, and the supernatant was removed. The cell pellet was resuspended in bead buffer (500 ul/10⁸ cells) and loaded onto a LS column placed in a MACS Separator. The column was washed 3× with 3 mL of buffer and then elution buffer was added and the magnetically labeled cells were flushed from the column by firmly applying the plunger supplied with the column. The eluted CD19+ cells were centrifuged at 300×g for 10 minutes, and resuspended in 10 ml of RPMI-1640 (with 10% FBS).

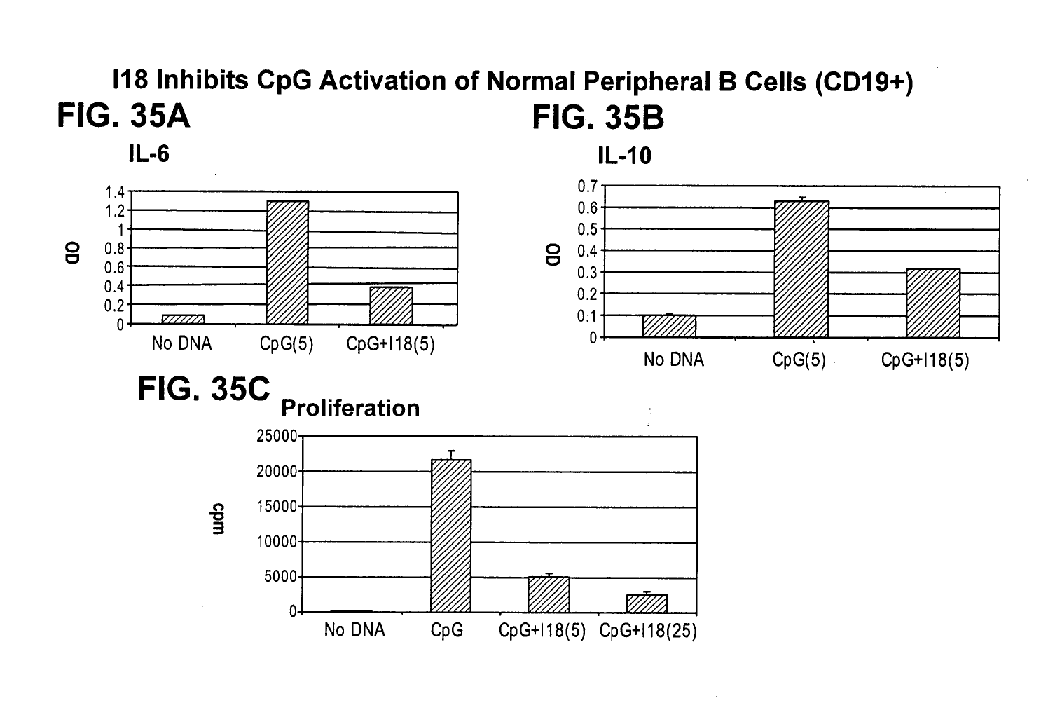

To determine the effect of I18 on CpG-ODN stimulated IL-6 and IL-10 cytokine production, CD19+ B cells were incubated for 48 hours with 5 μg/mL stimulatory CpG-ODN alone or in the presence of 5 μg/mL I18. Cytokine levels in the culture medium were analyzed by ELISA (Pharmingen, human IL-6, Cat #555220; human IL-10, Cat #555157) according to the manufacturer's protocol. As shown in FIG. 35, 118 suppressed both CpG stimulated IL-6 (FIG. 35A) and IL-10 (FIG. 35B) expression.

To determine if I18 could inhibit CpG-ODN stimulation of cell proliferation, CD19+ B cells were incubated with 5 μg/mL stimulatory CpG-ODN alone or in the presence of 5 μg/mL or 25 μg/mL I18 for 4 days. Cell proliferation was assayed by [³H] thymidine incorporation during the last 24 hrs of incubation. I18 significantly suppressed CpG stimulated C cell proliferation at both dosages (FIG. 35C).

### Example 14

### I18 Inhibits CpG Activation of Peripheral B Cells (CD19+) from a Lupus Patient

To determine the effect of I18 on lupus B cells activated by immune stimulatory CpG sequences, CD19+ peripheral B cells were isolated from a patient with SLE and cytokine production and proliferation were examined in the presence or absence of I18. The patient is a 23 year old female diagnosed with SLE less than one year ago who is taking Plaquenil.

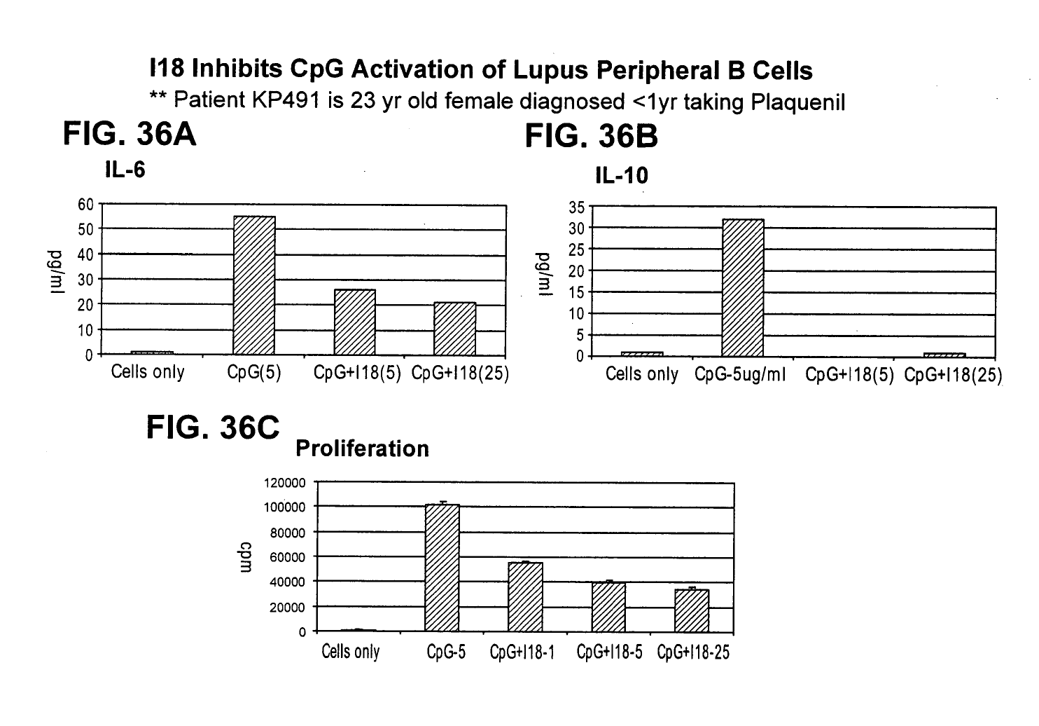

CD19+ B cells were isolated as described in detail above. The effect of I18 on CpG-ODN stimulated IL-6 and IL-10 cytokine production by lupus CD19+ B cells was examined by incubating cells for 48 hours with 5 μg/mL stimulatory CpG-ODN alone or in the presence of 5 μg/mL or 25 μg/mL I18. Cytokine levels in the culture medium were analyzed by ELISA as described above. As shown in FIG. 36, 118 suppressed both CpG stimulated IL-6 (FIG. 36A) and IL-10 (FIG. 36B) expression.

To determine if I18 could inhibit CpG-ODN stimulated proliferation of CD19+ B cells, cells were incubated with 5 μg/mL stimulatory CpG-ODN alone or in the presence of 1 μg/mL, 5 μg/mL or 25 μg/mL I18 for 4 days. Cell proliferation was assayed by [³H] thymidine incorporation during the last 24 hrs of incubation. I18 significantly suppressed CpG stimulated C cell proliferation at all dosages (FIG. 36C).

### Example 15

### I18 Activates Normal and Lupus B Cells

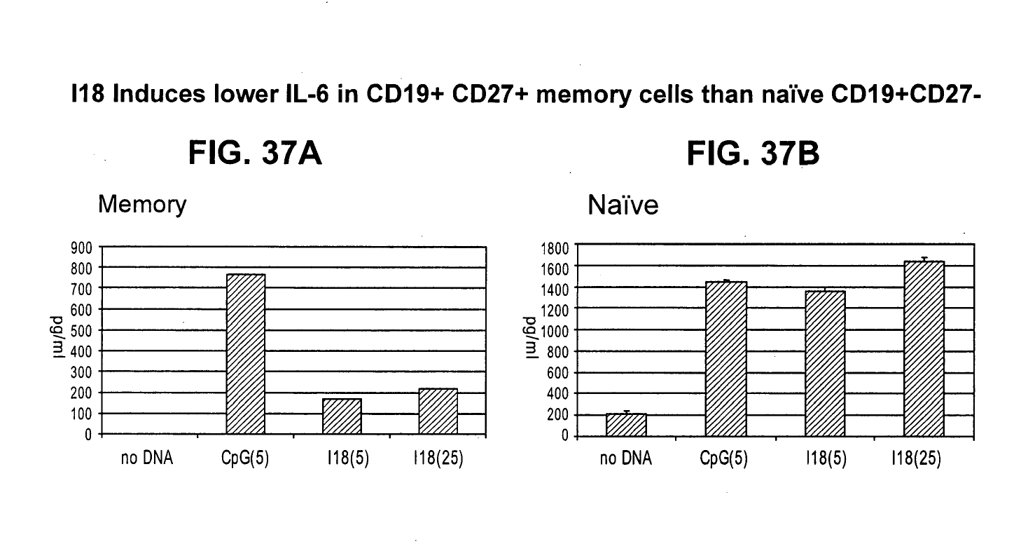

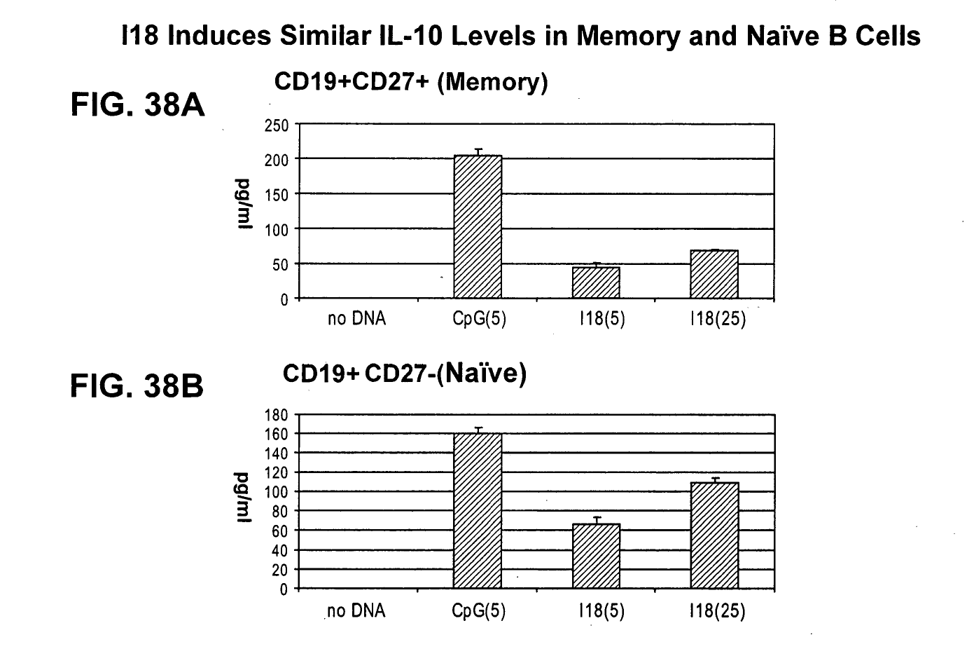

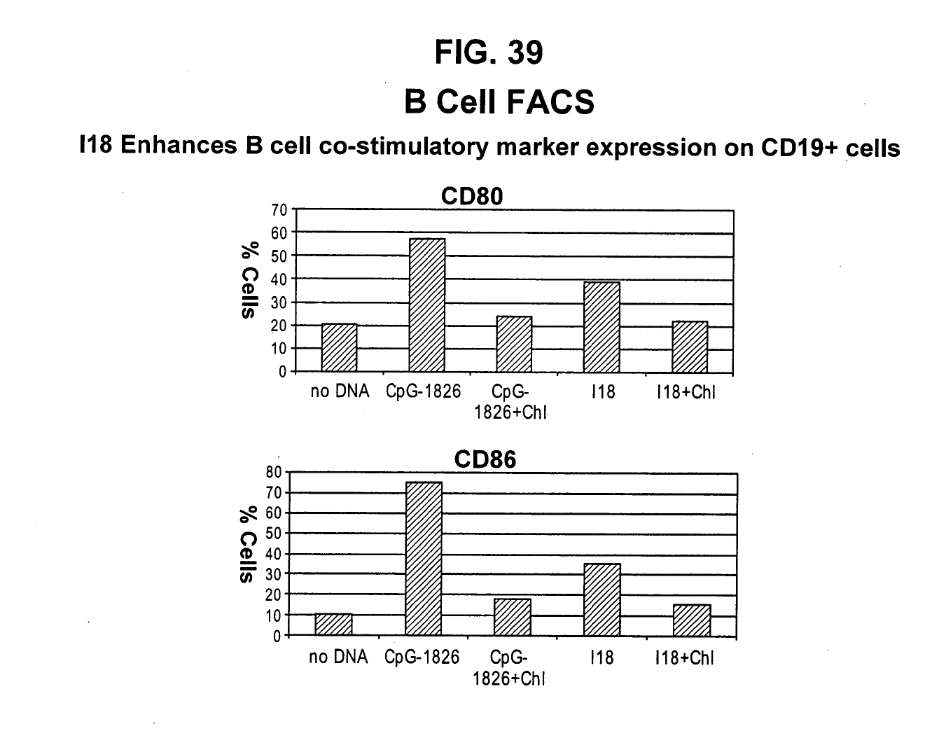

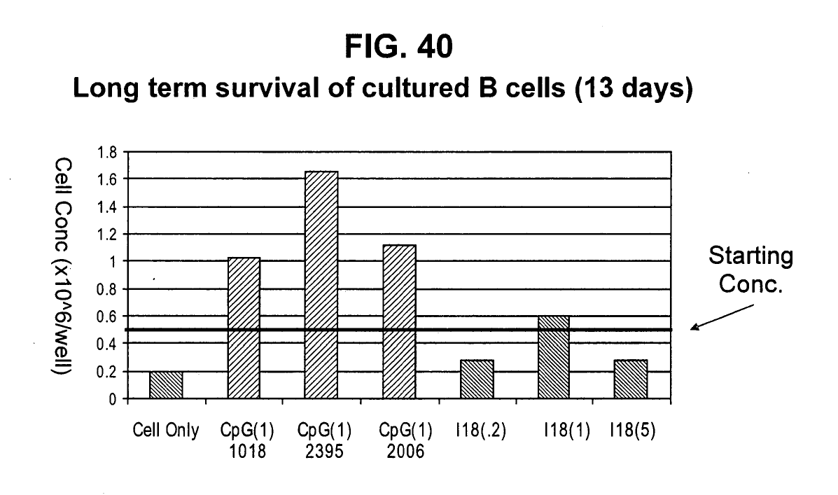

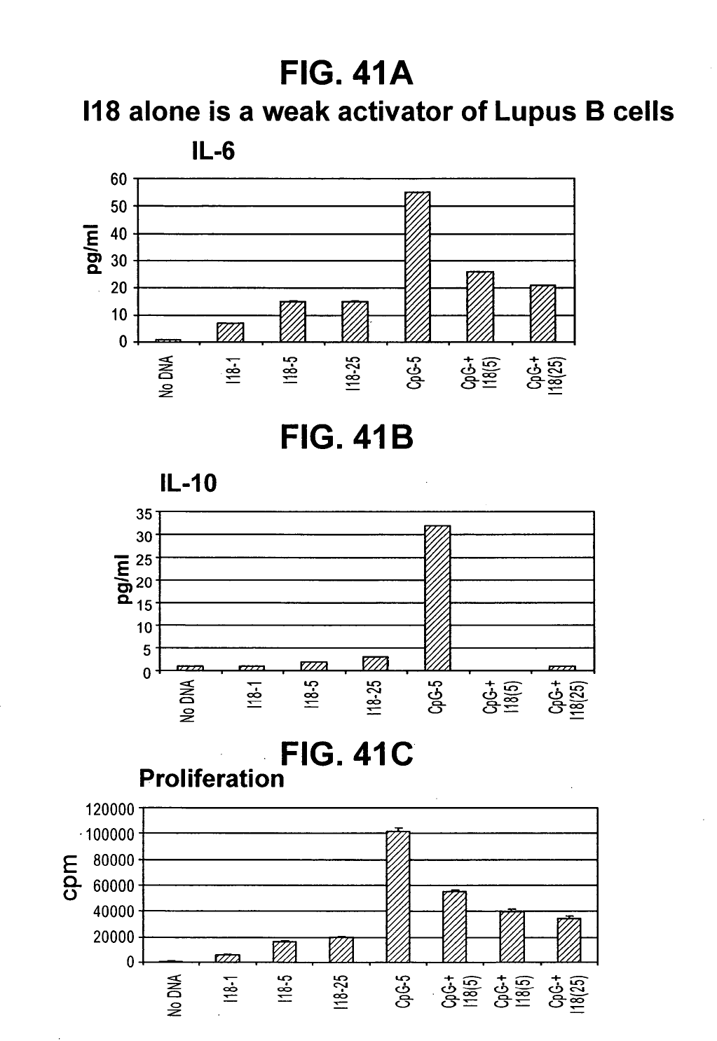

The effect of I18 on peripheral B cell activation was compared to immune stimulatory CpG sequences. Incubation of isolated CD19+CD27− naive B cells with 5 μg/mL or 25 μg/mL I18 induced IL-6 expression to a similar degree as CpG sequences (FIG. 37B). In contrast, 5 μg/mL or 25 μg/mL I18 incubated with isolated CD19+CD17+ memory B cells induced IL-6 expression to a much lesser degree than CpG sequences (FIG. 37A). I18 also induced IL-10 expression in both naïve and memory B cells at both 5 μg/mL and 25 μg/mL, though at lower levels than induced by CpG-ODN (FIG. 38). Similarly, I18 activated in a Chloroquine sensitive manner B cell co-stimulatory marker CD80 and CD86 expression at lower levels than CpG sequences as determined by FACS (FIG. 39). 118 did not, however, increase B cell survival or proliferation as did CpG sequences when B cells were cultured in 10% FBS with or without oligonucleotides for 13 days (FIG. 40). Finally, I18 was a much weaker activator of IL-6 (FIG. 41A), IL-10 (FIG. 41B) and cell proliferation (FIG. 41C) of B cells from a SLE patient.

### Example 16

### I18 Delays Disease Onset in a Mouse Model of SLE

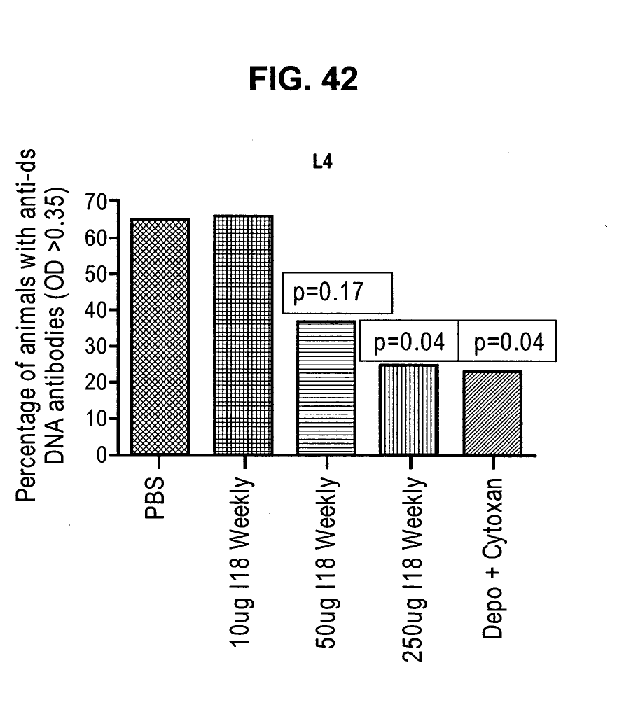

I18 oligos were tested for their ability to affect disease onset in an animal model of lupus. NZB/W F1 female mice spontaneously develop proteinurea, kidney pathology and antibodies to DNA similar to individuals with systemic lupus erythematosus (SLE). I18 IMS oligos were administered to NZB/W F1 female mice weekly at 10 μg, 50 μg and 250 μg by intradermal delivery. The percentage of animals with anti-dsDNA antibodies was statistically less in the groups receiving 50 μg (p=0.17) and 250 μg (p=0.04) weekly doses of I18 (FIG. 42).

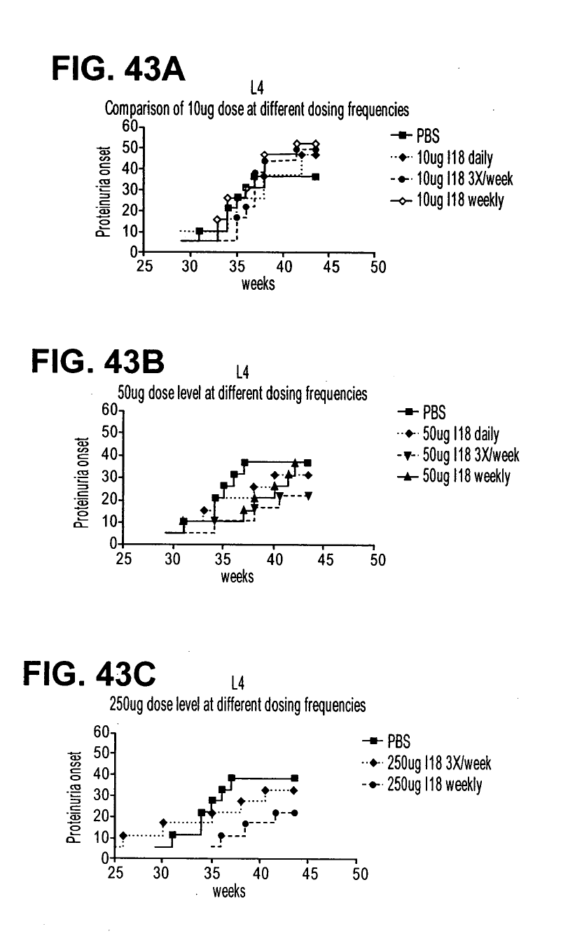

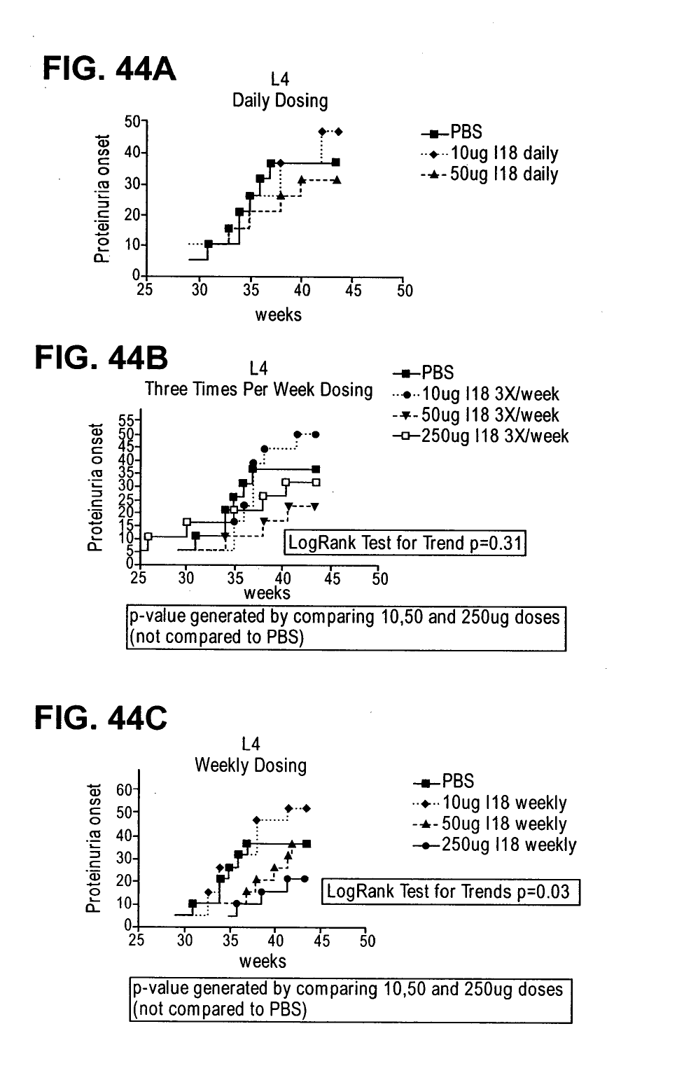

Next different dosage frequencies were examined. NZB/W F1 females were administered 10 μg, 50 μg, or 250 μg I18 daily, 3× weekly or weekly for a total of 45 weeks, and proteinuria onset was assessed. Administration of 10 μg I18 did not affect disease onset (FIG. 43A). In contrast, all dosing regimes at 50 μg and 250 μg showed a trend towards decreased disease onset compared to PBS controls (FIG. 43B, C). Importantly, both 3× weekly (FIG. 44B) and weekly (FIG. 44C) administration of 250 μg I18 showed a statistically significant trend (LogRank Test p=0.31 and p=0.03, respectively) compared to administration with 10 μg and 50 μg I18.

### Example 17

### Treatment of Human SLE with I18

The immunomodulatory oligonucleotide I18 is used to treat human SLE patients. Patients diagnosed with SLE are first screened for the presence of anti-dsDNA antibodies in their serum by ELISA. Patients presenting with anti-dsDNA antibodies are then treated with therapeutically effective amounts of I18 in the range of about 0.001 micrograms to about 1 gram. A preferred therapeutic amount of I18 is in the range of about 5 micrograms to about 500 micrograms. A most preferred therapeutic amount of I18 is in the range of about 50 to 200 micrograms. I18 therapy is delivered daily, every-other-day, twice-per-week, weekly, every-two-weeks or monthly on an ongoing basis. In a preferred therapeutic regime the I18 therapy is delivered monthly for between 6-12 months, and then every between 3-12 months as a maintenance dose. Human SLE patients monitored for disease activity.

### Example 18

### I18 and Related Oligonucleotides Inhibit CpG Stimulation of IL-6 by Human B Cells

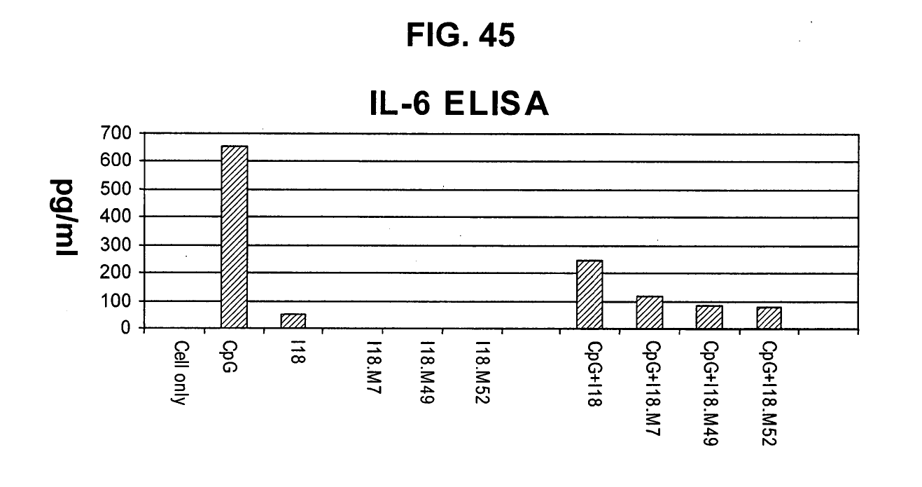

Mutagenesis of immunomodulatory oligonucleotide I18 identified five related oligonucleotides with enhanced immunomodulatory activity. Systematic alteration of I18 generated the related oligonucleotides: I18.M7 (CCATGTGGAAATGGGT); I18.M49 (CCATGTGGCCCTGGGT); I18.M51 (CCATGTGGAAAAGGGT); I18.M52 (CCATGTGGAAAAGGGA); I18.M53 (CCATGTGCCCAAGGGA). To determine the effect of I18-derived oligonucleotides on CpG-ODN stimulated IL-6 cytokine production, human B cells were incubated for 48 hours with 5 μg/mL stimulatory CpG-ODN or I18-derived oligonucleotides alone or 5 μg/mL stimulatory CpG-ODN in the presence of 5 μg/mL I18 or I18-derived oligonucleotides (FIG. 45). Cytokine levels in the culture medium were analyzed by ELISA (Pharmingen, human IL-6, Cat #555220) according to the manufacturer's protocol. Whereas incubation of human B cells with I18 resulted in a small stimulation of IL-6 production, none of the I18-derived oligonucleotides triggered detectable cytokine production (FIG. 1, left columns). Similarly, I18-derived oligonucleotides inhibited IL-6 production by CpG-ODN better than I18, though all immunomodulatory oligonucleotides resulted in statistically significant inhibition (FIG. 45, right columns).

### Example 19

### Characterization of Oligos with Distinct Levels of Immune Inhibitory and Stimulatory Properties

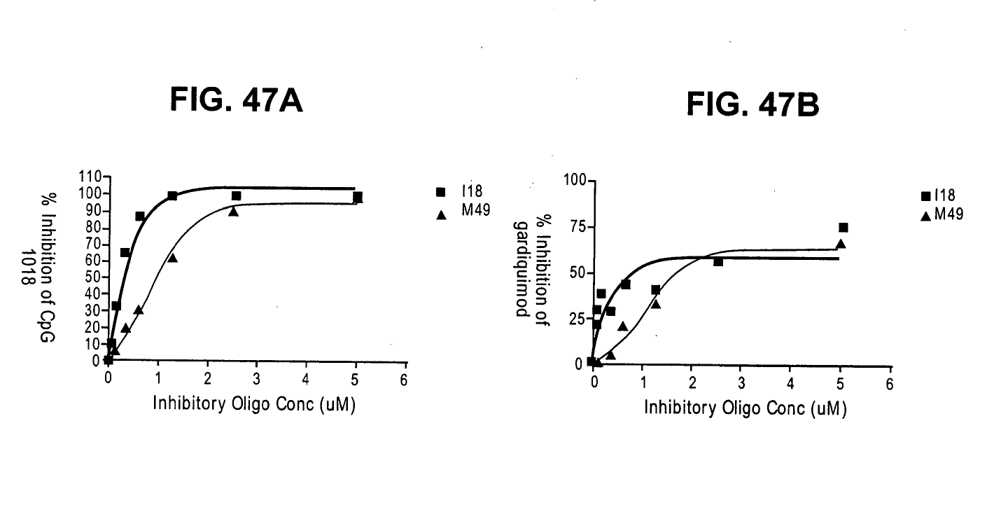

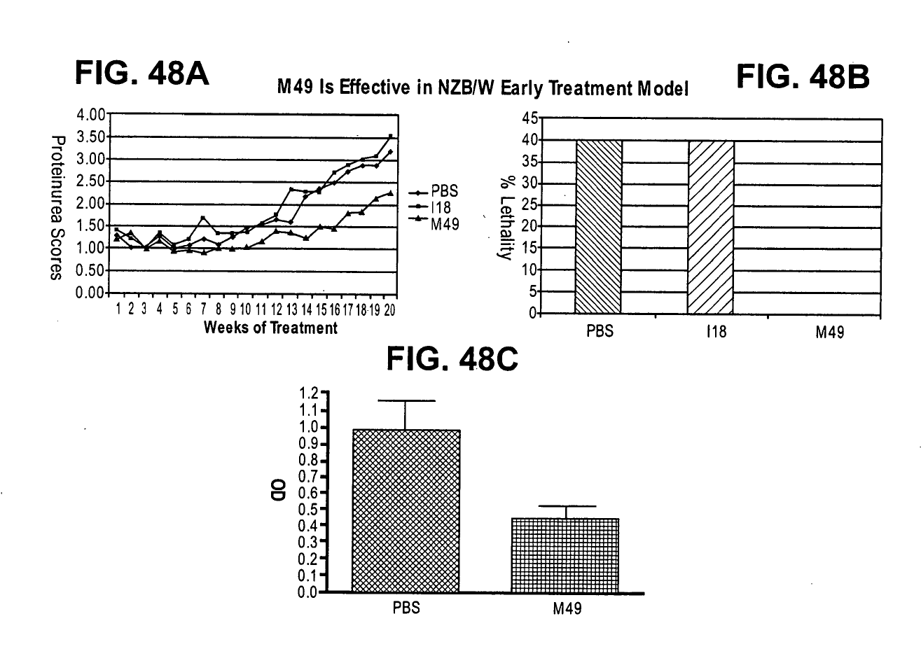

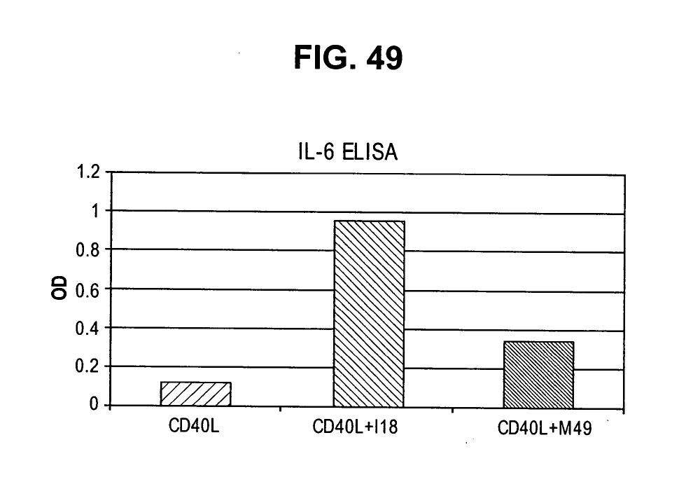

Inhibitory oligonucleotides were screened in assays to determine the relative levels of immune inhibitory and stimulatory activity possessed by each oligo. To determine inhibitory activity, mouse splencoytes were incubated with TLR7 and TLR9 agonists alone and in the presence of the inhibitory oligonucleotides and activation of inflammatory cytokines like IL-6 were measured (FIG. 47). To test for the presence of immune stimulatory properties human B cells were incubated with a combination of recombinant CD40 ligand and oligonucleotide and B cell activation was measured by examining cytokine production in short term cultures or survival and immunoglobulin production in long term cultures (FIG. 49). Oligos with distinct levels of activating and inhibitory activities were selected for further testing in animal models. Animal studies were performed using the NZB/W F1 strain. Oligonucleotides were delivered weekly by IP or subcutaneous routes and animals were assessed for survival, proteinurea levels, and the levels of anti-dsDNA antibodies (FIG. 48).

The previous examples are specific embodiments for carrying out the present invention. The examples are offered for illustrative purposes only, and are not intended to limit the scope of the present invention in any way. Other variants of the inventions will be readily apparent to those of ordinary skill in the art and encompassed by the appended claims. All publications, patents, patent applications, and other references cited herein are hereby incorporated by reference.

1. A pharmaceutical composition comprising:
(a) an immune modulatory nucleic acid comprising an immune modulatory sequence comprising:
(i) a hexameric sequence
5′-Purine-Pyrimidine_{[1]}-[X]-[Y]-Pyrimidine_{[2]}-Pyrimidine_{[3]}-3;
wherein X and Y are any naturally occurring or synthetic nucleotide, except that:
a. X and Y cannot be cytosine-guanine;
b. X and Y cannot be cytosine-cytosine when Pyrimidine_{[2]}is thymine
c. X and Y cannot be cytosine-thymine when Pyrimidine_{[1]} is cytosine;
(ii) a CC dinucleotide 5′ to the hexameric sequence wherein the CC dinucleotide is between one to five nucleotides 5′ of the hexameric sequence; and
(iii) a polyG region 3′ of the hexameric sequence wherein the polyG comprises at least three contiguous Gs and is between two to five nucleotides 3′ of the hexameric sequence
wherein the immune modulatory sequence does not contain cytosine-guanine sequences and
(b) a pharmaceutically acceptable carrier.

2. The pharmaceutical composition of claim 1, wherein the CC dinucleotide is two nucleotides 5′ of the hexameric sequence.

3. The pharmaceutical composition of claim 1, wherein the polyG region is two nucleotides 3′ of the hexameric sequence.

4. The pharmaceutical composition of claim 1, wherein the CC dinucleotide is two nucleotides 5′ of the hexameric sequence and the polyG region is two nucleotides 3′ of the hexameric sequence.

5. The pharmaceutical composition of claim 1, wherein X and Y of the hexameric sequence are guanine-guanine.

6. The pharmaceutical composition of claim 1, wherein the immune modultory nucleic acid is an oligonucleotide.

7. The pharmaceutical composition of claim 1, wherein the immune modultory nucleic acid is incorporated into a vector.

8. The pharmaceutical composition of claim 7, wherein the vector is an expression vector.

9. A pharmaceutical composition comprising:
(a) an immune modulatory nucleic acid comprising an immune modulatory sequence comprising:
(i) a hexameric sequence
5′-Purine-Pyrimidine_{[1]}-[X]-[Y]-Pyrimidine_{[2]}-Pyrimidine_{[3]}-3′
wherein X and Y are guanine-guanine
(ii) a CC dinucleotide 5′ to the hexameric sequence wherein the CC dinucleotide is between one to five nucleotides 5′ of the hexameric sequence; and
(iii) a polyG region 3′ of the hexameric sequence wherein the polyG comprises at least three contiguous Gs and is between two to five nucleotides 3′ of the hexameric sequence
wherein the immune modulatory sequence does not contain cytosine-guanine sequences and
(b) a pharmaceutically acceptable carrier.

10. The pharmaceutical composition of claim 9, wherein the CC dinucleotide is two nucleotides 5′ of the hexameric sequence.

11. The pharmaceutical composition of claim 9, wherein the polyG region is two nucleotides 3′ of the hexameric sequence.

12. The pharmaceutical composition of claim 9, wherein the CC dinucleotide is two nucleotides 5′ of the hexameric sequence and the polyG region is two nucleotides 3′ of the hexameric sequence.

13. The pharmaceutical composition of claim 9, wherein the hexameric sequence is GTGGTT.

14. The pharmaceutical composition of claim 9, wherein the hexameric sequence is GTGGTT, the CC dinucleotide is two nucleotides 5′ of the hexameric sequence and the polyG region is two nucleotides 3′ of the hexameric sequence.

15. The pharmaceutical composition of claim 9, wherein the hexameric sequence is GTGGTT and the CC dinucleotide is two nucleotides 5′ of the hexameric sequence.

16. The pharmaceutical composition of claim 9, wherein the hexameric sequence is GTGGTT and the polyG region is two nucleotides 3′ of the hexameric sequence.

17. The pharmaceutical composition of claim 9, wherein the immune modulatory sequence is CCATGTGGTTATGGGT (SEQ ID NO:73).

18. The pharmaceutical composition of claim 9, wherein the immune modulatory nucleic acid is an oligonucleotide.

19. The pharmaceutical composition of claim 9, wherein the immune modulatory nucleic acid is incorporated into a vector.

20. The pharmaceutical composition of claim 19, wherein the vector is an expression vector.

21. A method for treating a disease in a subject associated with one or more self-molecules present non-physiologically in the subject, the method comprising: administering to the subject an immune modulatory sequence comprising an immune modulatory sequence comprising:
(i) a hexameric sequence
5′-Purine-Pyrimidine_{[1]}-[X]-[Y]-Pyrimidine_{[2]}-Pyrimidine_{[3]}-3;
wherein X and Y are any naturally occurring or synthetic nucleotide, except that:
d. X and Y cannot be cytosine-guanine;
e. X and Y cannot be cytosine-cytosine when Pyrimidine_{[2]} is thymine
f. X and Y cannot be cytosine-thymine when Pyrimidine_{[1]} is cytosine;
(ii) a CC dinucleotide 5′ to the hexameric sequence wherein the CC dinucleotide is between one to five nucleotides 5′ of the hexameric sequence; and
(iii) a polyG region 3′ of the hexameric sequence wherein the polyG comprises at least three contiguous Gs and is between two to five nucleotides 3′ of the hexameric sequence
wherein the immune modulatory sequence does not contain cytosine-guanine sequences.

22. The method of claim 21, wherein the CC dinucleotide is two nucleotides 5′ of the hexameric sequence.

23. The method of claim 21, wherein the polyG region is two nucleotides 3′ of thehexameric sequence.

24. The method of claim 21, wherein the CC dinucleotide is two nucleotides 5′ of the hexameric sequence and the polyG region is two nucleotides 3′ of the hexameric sequence.

25. The method of claim 21, wherein X and Y of the hexameric sequence are guanine-guanine.

26. The method of claim 21, wherein the hexameric sequence is GTGGTT.

27. The method of claim 21, wherein the hexameric sequence is GTGGTT, the CC dinucleotide is two nucleotides 5′ of the hexameric sequence and the polyG region is two nucleotides 3′ of the hexameric sequence.

28. The method of claim 21, wherein the hexameric sequence is GTGGTT and the CC dinucleotide is two nucleotides 5′ of the hexameric sequence.

29. The method of claim 21, wherein the hexameric sequence is GTGGTT and the polyG region is two nucleotides 3′ of the hexameric sequence.

30. The method of claim 21, wherein the nucleotide sequence is CCATGTGGTTATGGGT (SEQ ID NO:73).

31. The method of claim 21, wherein the immune modulatory nucleic acid is an oligonucleotide.

32. The method of claim 21, wherein the immune modulatory nucleic acid is incorporated into a vector.

33. The method of claim 32, wherein the vector is an expression vector.

34. The method of claim 21, wherein the disease associated with one or more self-molecules present non-physiologically in the subject is systemic lupus erythematosus.

35. A method for treating a disease in a subject associated with one or more self-molecules present non-physiologically in the subject, the method comprising: administering to the subject an immune modulatory sequence comprising an immune modulatory sequence comprising:
i) a hexameric sequence
5′-Purine-Pyrimidine_{[1]}-[X]-[Y]-Pyrimidine_{[2]}-Pyrimidine_{[3]}-3′
wherein X and Y are guanine-guanine
(ii) a CC dinucleotide 5′ to the hexameric sequence wherein the CC dinucleotide is between one to five nucleotides 5′ of the hexameric sequence; and
(iii) a polyG region 3′ of the hexameric sequence wherein the polyG comprises three at least three contiguous Gs and is between two to five nucleotides 3′ of the hexameric sequence
wherein the immune modulatory sequence does not contain cytosine-guanine sequences.

36. The method of claim 35, wherein the CC dinucleotide is two nucleotides 5′ of the hexameric sequence.

37. The method of claim 35, wherein the polyG region is two nucleotides 3′ of the hexameric sequence.

38. The method of claim 35, wherein the CC dinucleotide is two nucleotides 5′ of the hexameric sequence and the polyG region is two nucleotides 3′ of the hexameric sequence.

39. The method of claim 35, wherein the hexameric sequence is GTGGTT.

40. The method of claim 35, wherein the hexameric sequence is GTGGTT and the CC dinucleotide is two nucleotides 5′ of the hexameric sequence.

41. The method of claim 35, wherein the hexameric sequence is GTGGTT and the polyG region is two nucleotides 3′ of the hexameric sequence.

42. The method of claim 35, wherein the hexameric sequence is GTGGTT, the CC dinucleotide is two nucleotides 5′ of the hexameric sequence and the polyG region is two nucleotides 3′ of the hexameric sequence.

43. The method of claim 35, wherein the nucleotide sequence is CCATGTGGTTATGGGT (SEQ ID NO:73).

44. The method of claim 35, wherein the immune modulatory nucleic acid is an oligonucleotide.

45. The method of claim 35, wherein the immune modulatory nucleic acid is incorporated into a vector.

46. The method of claim 35, wherein the vector is an expression vector.

47. The method of claim 35, wherein the disease associated with one or more self-molecules present non-physiologically in the subject is systemic lupus erythematosus.
